(19) United States
(12) Patent Application Publication
(10) Pub. No.: US 2026/0077068 A1
(43) Pub. Date: Mar. 19, 2026

(54) ATP-INDEPENDENT BIOLUMINESCENT REPORTER VARIANTS TO IMPROVE IN VIVO IMAGING

(71) Applicant: University of Virginia Patent Foundation, Charlottesville, VA (US)

(72) Inventors: Huiwang Ai, Crozet, VA (US); Xiadong Tian, Charlottesville, VA (US); Shengyu Zhao, Ansonia, CT (US); Ying Xiong, Rockville, MD (US)

(21) Appl. No.: 19/347,609

(22) Filed: Oct. 1, 2025

Related U.S. Application Data
(63) Continuation-in-part of application No. 17/434,351.
(60) Provisional application No. 62/811,129, filed on Feb. 27, 2019.
(60) Provisional application No. 63/701,942, filed on Oct. 1, 2024.

(51) Int. Cl. A61K 49/00; C07D 487/04; C12N 9/02

(52) U.S. Cl. CPC A61K 49/0052; C07D 487/04; C12N 9/0069; C12Y 113/12013; C07K 2319/00

(57) ABSTRACT

Disclosed are ATP-independent bioluminescent reporter systems for enhanced in vivo imaging. Also disclosed is a modified luciferin, potassiorin, comprising a potassium-binding moiety. Also disclosed is an engineered luciferase, BRIPO, optimized for synergistic interaction with potassiorin. A further version of the disclosed system produces bioluminescence signals responsive to physiological potassium concentrations. In further instances, the system enables real-time monitoring of K+ dynamics in live cells, tissues, and animals. A further embodiment includes applications in imaging neuronal activity, studying ion flux, and developing bioluminescent indicators for diverse analytes. The disclosed system addresses limitations of traditional imaging methods, offering improved sensitivity and biocompatibility.

### CROSS REFERENCE TO RELATED APPLICATIONS

This application claims the benefit of U.S. Provisional Pat. App. No. 63/701,942 filed 1 Oct. 2024. This Application is a further Continuation-in-Part of U.S. patent application Ser. No. 17/434,351 filed 26 Aug. 2021 which further claims the benefit of U.S. Provisional Pat. App. No: 62/811,129, filed 27 Feb. 2019, the disclosures of which are incorporated herein by reference in their entirety.

### GRANT STATEMENT

This invention was made with government support under EB035430, GM118675 and GM129291 awarded by the National Institutes of Health, and CHE1750660 awarded by the National Science Foundation. The government has certain rights in the invention.

### SEQUENCE LISTING

A sequence listing in ST.26 is being submitted herewith electronically, the name of the file is UVA0021USP, the date of creation is Oct. 22, 2025, and the size of the file is 83,035 bytes (81.0 kb). The sequence listing contains a total of fifty (50) sequences and is hereby incorporated by reference in its entirety.

### TECHNICAL FIELD

The presently disclosed subject matter relates to ATP-independent bioluminescent reporter variants to improve in vivo imaging.

Embodiments of the disclosure presented herein relate generally to the field of bioluminescent indicators. In certain embodiments there is a disclosed a modified luciferin molecule with an analyte-binding moiety. Particular embodiments disclose bioluminescent indicators for potassium (K⁺) ions. Certain embodiments disclose a K⁺-binding luciferin named potassiorin and a method of synthesis for the compound. In another embodiment, there is disclosed an engineered luciferase enzyme called BRIPO (bioluminescent red indicator for potassium) engineered to work synergistically with potassiorin, resulting in optimized K-dependent bioluminescence responses.

In some embodiments, methods, kits, and compositions for enhanced bioluminescent imaging are provided.

### BACKGROUND

In the past few decades, fluorescence imaging has evolved quickly and become a dominant visualization method for live-cell studies. However, fluorescence imaging has several limitations, such as photobleaching, phototoxicity, and poor tissue penetration, largely due to the need for light excitation. Unlike fluorescence, bioluminescence produces photons via enzyme-catalyzed biochemical reactions in which luciferases oxidize their corresponding small-molecule substrates (also referred to as luciferins) to generate excited-state emitters. As a result, bioluminescence signals glow essentially on dark background, leading to excellent signal-to-background ratios. Moreover, even though the spatiotemporal resolution of bioluminescence imaging (BLI) is usually worse than that of fluorescence imaging, the emitted photons can escape through several centimeters of tissue. BLI is thus especially suited for diverse, noninvasive in vivo imaging applications.

Photinus pyralis firefly luciferase (FLuc) and D-luciferin (λmax: 563 nm) constitute the most widely used luciferase-luciferin pair for in vivo BLI. Recently, research has been performed to develop FLuc and D-luciferin derivatives for brighter and more red-shifted emission. In particular, an Akaluc-AkaLumine luciferase-luciferin pair with near-infrared (NIR) emission (λmax: 650 nm) was reported for highly sensitive deep-tissue in vivo BLI. Despite the progress, AkaLumine has been shown to induce cytotoxicity. Moreover, FLuc, Akaluc, and other insect luciferases consume ATP for photon production; the bioluminescence reaction between FLuc and D-luciferin reduced the intracellular ATP-to-ADP ratio of live mammalian cells from greater than 40:1 to about 20:1, suggesting metabolic disruption by all ATP-dependent luciferases. Because ATP is required for the activation of the luciferin substrates, this metabolic disruption issue cannot be addressed by simply improving insect luciferases and the corresponding substrates.

In contrast to insect luciferases, a large family of marine luciferases and photoproteins, such as Renilla luciferase (RLuc), Gaussia luciferase (GLuc), Oplophorus luciferase (OLuc), and aequorin, are ATP-independent and use coelenterazine (CTZ) as their native substrate for bioluminescence production. The 19 kDa catalytic domain of OLuc was recently engineered into NanoLuc, which has a fast enzyme turnover and produces intense blue bioluminescence (λmax: 456 nm) in the presence of a synthetic CTZ analog, furimazine (FRZ). To expand the color palette, NanoLuc was further engineered into teLuc, which emits red-shifted photons (λmax: 502 nm) when paired with a synthetic substrate diphenylterazine (DTZ). Since biological tissues significantly absorb and scatter short-wavelength photons, NanoLuc and teLuc have been fused to fluorescent proteins, resulting in Antares, Antares2, and enhanced Nano-Lanterns for further red-shifted emission via bioluminescence resonance energy transfer (BRET). The water solubility of CTZ, FRZ, and DTZ is adequate for protein- and cell-based assays, because their solubility is already higher than typical substrate concentrations in these in vitro assays. However, in vivo applications of existing ATP-independent bioluminescent reporters are greatly hindered by the low solubility of CTZ, FRZ, and DTZ. Small animals, such as mice, can only tolerate a small injection volume. To enhance bioluminescence brightness by delivering more luciferin substrate, hydroxypropyl-β-cyclodextrin, polyethylene glycols (PEGs), or other organic cosolvents are typically used to formulate CTZ, FRZ, or DTZ for in vivo administration. These formulation ingredients are not biologically inert and can cause irritation or biotoxicity. It is also practically difficult to intravenously inject these highly viscous solutions into small animals. Furthermore, it is still of great interest to further red-shift marine luciferases for enhanced tissue penetration.

The NanoLuc system derived from the deep-sea shrimp Oplophorus gracilirostris has gained significant attention. This system offers additional advantages such as a small protein size, high enzyme stability, and a remarkable >150-fold increase in luminescence compared to traditional luciferases. In the presence of the synthetic luciferin furimazine, NanoLuc emits intense blue photons at around 450 nm. Further research has led to the development of red-shifted variants of NanoLuc, achieved through redesigned synthetic luciferins or fusion with red-emitting fluorescent proteins (FPs) for resonance energy transfer (RET).

For indicator development, NanoLuc is frequently utilized as a RET donor. It has been fused with sensory domains and a RET acceptor to achieve RET efficiency modulation and ratiometric responses. However, these RET-based indicators face challenges in achieving a wide dynamic range and are better suited for in vitro assays rather than in vivo BLI applications due to the strong attenuation of NanoLuc's blue emission by mammalian tissue.

Another promising approach involves directly incorporating sensory domains into the structure of NanoLuc or its derived luciferases (FIG. 23, b). This results in the modulation of luciferase activity through structural changes upon analyte binding. In certain cases, the luciferases are further fused with red-emitting FPs to achieve red-shifted emission for better tissue penetration. This strategy has led to the development of several bioluminescent sensors for calcium ions (Ca²⁺) and neurotransmitters. However, this approach necessitates the identification of appropriate protein-based sensory domains for specific analytes of interest, and the engineering process can be tedious with unpredictable outcomes.

Taken together, there is a long-felt need for new and improved ATP-independent bioluminescent reporters, which have improved biochemical and photophysical properties and broad applicability. There is a further continued need to expand the strategies for generating bioluminescent indicators; and, in particular, for indicators that expand the capability of monitoring ions in living systems. Such needs are addressed by the present disclosure.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, provided herein are bioluminescent proteins, comprising a substituted luciferase polypeptide comprising an amino acid sequence having at least 90% homology to SEQ ID NO: 2 with amino acid substitutions at one or more of positions corresponding to positions 6, 20, 21, 29, 30, 69, 73, 87, 92, 114, 121, and 138 of SEQ ID NO: 2. In some embodiments, the bioluminescent proteins comprise amino acid substitutions at positions corresponding to positions 20, 21, 29, and 30, and further comprise one or more amino acid substitutions at one or more of positions corresponding to positions 6, 69, 73, 87, 92, 114, 121, and 138 of SEQ ID NO: 2. In some embodiments, the bioluminescent protein comprise an amino acid sequence having at least 95% homology to SEQ ID NO: 2 with amino acid substitutions at least eight positions selected from positions corresponding to positions 6, 20, 21, 29, 30, 69, 73, 87, 92, 114, 121, and 138 of SEQ ID NO: 2. In some embodiments, a luciferase polypeptide is substituted at positions corresponding to positions 6, 20, 21, 29, 30, 69, 73, 87, 92, 114, 121, and 138 of SEQ ID NO: 2. In some embodiments, the luciferase polypeptide can comprise SEQ ID NO: 3.

In some embodiments, bioluminescent proteins disclosed herein can comprise a fluorescent protein connected to the substituted luciferase polypeptide. The fluorescent protein can be connected to the substituted luciferase polypeptide so as to allow bioluminescence resonant energy transfer (BRET) between the substituted luciferase polypeptide and the fluorescent protein. A substituted luciferase polypeptide can comprise an amino acid sequence having at least 90% homology to SEQ ID NO: 5, or can comprise the amino acid sequence of SEQ ID NO: 5.

Provided herein are bioluminescent proteins comprising an amino acid sequence having at least 90% homology to SEQ ID NO: 6, SEQ ID NO: 8, SEQ ID NO: 10, or SEQ ID NO: 12. Nucleic acids encoding these bioluminescent proteins are also provided. Vectors encoding the nucleic acids are provided, as are expression vectors encoding the nucleic acids and functionally connected to a promoter. In some aspects, cell lines containing the expression vector and expressing the bioluminescent protein are provided. Additionally, non-human cells transfected with the expression vector and expressing the bioluminescent protein are provided.

Combinations comprising a bioluminescent protein and a luciferin are provided. The luciferin can comprise a luciferin selected from pyCTZ, 6pyDTZ, and 8pyDTZ. Methods of producing luminescence, comprising reacting a luciferin with a disclosed bioluminescent protein, are also provided. In such methods the bioluminescent protein can further comprise a fluorescent protein connected to the substituted luciferase polypeptide so as to allow BRET activity between the substituted luciferase polypeptide and the fluorescent protein.

Provided herein are luciferin compounds comprising an analog of pyridyl coelenterazine (CTZ), pyridyl furimazine (FRZ) and/or a pyridyl diphenylterazine (DTZ). The compounds comprise a water solubility increased by at least about 4-fold as compared to CTZ and/or DTZ, optionally by at least about 10-fold as compared to CTZ and/or DTZ. The compounds can in some aspects comprise a water solubility ranging from about 1,000 μM to about 1,800 μM. The luciferin compounds can comprise a bioluminescence ranging from about 450 nm to about 550 nm, and can be compatible with any luciferase, optionally any ATP-independent luciferase, including for example luciferases comprising a polypeptide comprising SEQ ID NO: 3, SEQ ID NO: 5, SEQ ID NO:6, SEQ ID NO:8, SEQ ID NO: 10, or SEQ ID NO: 12.

Methods of making a luciferin compound are provided herein, and can include making one or more pyridyl isomer substitutions at a C-2, C-6 and C-8 position of an imidazopyrazinone core.

In some embodiments, provided herein are bioluminescent reporter systems, such systems comprising at least two bioluminescent proteins selected from RLuc8, Opy Luc and Akaluc, and at least two luciferin compounds select from pyOMeCTZ, pyDTZ and AkaLumine. In some aspects, the system comprises bioluminescent proteins consisting of RLuc8, OpyLuc and Akaluc, and luciferin compounds consisting of pyOMeCTZ, pyDTZ and AkaLumine. In some aspects, the bioluminescent proteins and luciferin compounds are provided in pairs as follows: RLuc8 with pyOMeCTZ, Opy Luc with pyDTZ, and Akaluc with AkaLumine. The pairs of bioluminescent proteins and luciferins produce distinct colors of emission across the visible spectrum. In some aspects, RLuc8 paired with pyOMeCTZ yields a bioluminescence of about 380-470 nm, Opy Luc paired with pyDTZ yields a bioluminescence of about 480-600 nm, and Akaluc paired with AkaLumine yields a bioluminescence of about 600-750 nm. In such reporter systems, RLuc8 can comprise a polypeptide sequence comprising SEQ ID NO: 14, OpyLuc can comprise a polypeptide sequence comprising SEQ ID NO: 6, and Akaluc can comprise a polypeptide sequence comprising SEQ ID NO: 16. Nucleic acids, vectors, expression vectors and cell lines encoding and/or expressing the reporter systems are also provided.

In some embodiments, provided herein are methods of monitoring bioluminescence in a subject, the methods comprising providing a subject, establishing in the subject a luciferase expressing cell, wherein the luciferase is selected from the group consisting of LumiLuc, teLuc, RLuc8 and Opy Luc, administering to the subject a luciferin selected from the group consisting of pyCTZ, pyOHCTZ, pyOMeCTZ, pyOEtCTZ, pyiPrCTZ, 2pyDTZ, 6py DTZ, 60pyDTZ and 8pyDTZ, and measuring bioluminescence in the subject. In some aspects, the subject comprises a tumor or a cancer cell. In some aspects, the luciferase expressing cell comprises a tumor or a cancer cell. In some embodiments, the subject comprises an in vivo tumor or cancer model. In some embodiments, the subject is a non-human animal. In some embodiments, the bioluminescence is measured in a tumor or cancer cell in the subject.

Provided herein are cells containing an expression vector for expressing a bioluminescent protein, wherein the bioluminescent protein is selected from the group consisting of LumiLuc, teLuc, RLuc8 and OpyLuc, wherein the expression vector comprises a responsive promoter element, wherein the responsive promoter element is responsive to a stimuli of a cell signaling pathway. Such cells can be cultured in vitro.

Provided herein are cell signaling assay systems, the systems comprising cells containing an expression vector for expressing a bioluminescent protein, and a luciferin selected from the group consisting of pyCTZ, pyOHCTZ, pyOMeCTZ, pyOEtCTZ, pyiPrCTZ, 2pyDTZ, 6pyDTZ, 60pyDTZ and 8pyDTZ, wherein the assay is configured to indicate activation of a cell signaling pathway by bioluminescence.

Methods for monitoring a cell signaling pathway are provided, where the methods comprise culturing cells containing an expression vector for expressing a bioluminescent protein, exposing the cell to a luciferin selected from the group consisting of pyCTZ, pyOHCTZ, pyOMeCTZ, pyOEtCTZ, pyiPrCTZ, 2pyDTZ, 6pyDTZ, 60pyDTZ and 8pyDTZ, exposing the cell to a compound of interest, and measuring bioluminescence in the cell, wherein an increase in bioluminescence in the cell is indicative of an activation of the cell signaling pathway in the cell. The compound of interest can comprise a drug candidate compound.

Provided herein are stable cell lines integrated with a series of nucleic acids disclosed herein and expressing the bioluminescent proteins.

Disclosed are methods of generating bioluminescent indicators. In embodiments, there is disclosed a method of modifying luciferins with sensory moieties. In particular embodiments there is disclosed the design and synthesis of an artificial luciferin which selectively binds to potassium (K⁺) ions. The artificial luciferin is named potassiorin. Still other embodiments present the engineering of an artificial luciferase named BRIPO (Bioluminescent Red Indicator for Potassium) to work in conjunction with potassiorin, producing bioluminescence signals responsive to the physiological concentrations of K⁺. The methods and embodiments disclosed herein advantageously provide a powerful approach for the design of novel bioluminescent indicators.

In certain embodiments there is a bioluminescent indicator or metal ion salt thereof of the formula:

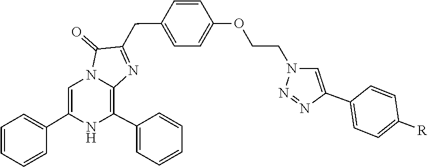

where R is

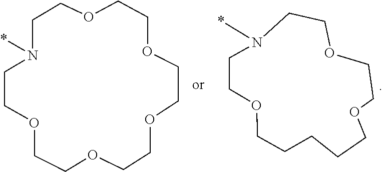 [SMILES: *N1CCOCCOCCOCCOCCOCC1 ; *N1CCOCCCCCOCCOCC1]

In still other embodiments, the indicator is a monopotassium salt of

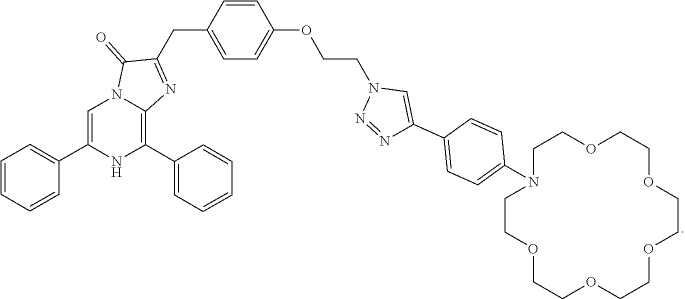 [SMILES: O=c1c(Cc2ccc(OCCn3cc(-c4ccc(N5CCOCCOCCOCCOCCOCC5)cc4)nn3)cc2)nc2c(-c3ccccc3)[nH]c(-c3ccccc3)cn1-2]

Certain other embodiments are an engineered luciferase protein of SEQ ID NO: 46 (BREP), SEQ ID NO: 47 (BRIPO0.5), or SEQ ID NO: 48 (BRIPO) or a protein having 95% homology with a protein of SEQ ID NO: 46 (BREP), SEQ ID NO: 47 (BRIPO0.5), or SEQ ID NO: 48.

Still other embodiments pertain to a method of producing a bioluminescent signal comprising contacting a monopotassium salt of a bioluminescent indicator of the formula

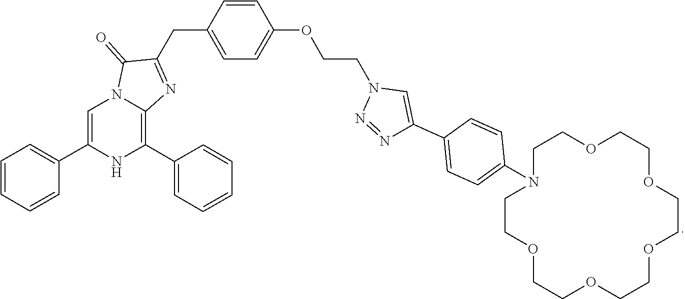 [SMILES: O=c1c(Cc2ccc(OCCn3cc(-c4ccc(N5CCOCCOCCOCCOCCOCC5)cc4)nn3)cc2)nc2c(-c3ccccc3)[nH]c(-c3ccccc3)cn1-2]

With an engineered luciferase protein of SEQ ID NO: 46 (BREP), SEQ ID NO: 47 (BRIPO0.5), or SEQ ID NO: 48 (BRIPO) or a protein having 95% homology with a protein of SEQ ID NO: 46 (BREP), SEQ ID NO: 47 (BRIPO0.5), or SEQ ID NO: 48.

The present disclosure provides novel bioluminescent indicators and related compositions, proteins, and methods for sensitive and selective detection of analytes in biological systems. In one aspect, the disclosure provides a bioluminescent indicator or a metal ion salt thereof having a defined chemical structure, wherein the indicator may be, for example, a monopotassium salt. The invention further provides engineered luciferase proteins, including but not limited to those having the amino acid sequences of SEQ ID NO: 46 (BREP), SEQ ID NO: 47 (BRIPO0.5), or SEQ ID NO: 48 (BRIPO), as well as proteins having at least 95% homology to any of these sequences.

Methods are provided for producing a bioluminescent signal by contacting a monopotassium salt of a bioluminescent indicator with an engineered luciferase protein as described above. In certain embodiments, the bioluminescent indicator, when contacted with the engineered luciferase protein BRIPO, exhibits at least a six-fold reduction in bioluminescence intensity upon exposure to 150 mM potassium ions, relative to the intensity observed in the absence of potassium ions. The indicators described herein may exhibit an apparent dissociation constant (Kd) for potassium ions in the range of about 1 mM to about 100 mM, and an apparent dissociation constant (Kd) for sodium ions of at least 50 mM.

The disclosure further provides bioluminescent calcium indicator compositions comprising a genetically engineered luciferase fusion protein that includes a NanoLuc-derived luciferase and a red fluorescent protein, such as mScarlet-I, with at least 80% sequence identity to SEQ ID NO: 49 or SEQ ID NO: 50. The luciferase fusion protein is engineered to include a calcium-binding domain that modulates bioluminescence in response to calcium ion concentration, and is used in combination with a luciferin substrate, such as diphenylterazine (DTZ) or a water-soluble derivative thereof. Upon binding of calcium ions, these indicators exhibit an increase in red-shifted bioluminescence emission suitable for in vivo imaging of calcium dynamics in mammalian cells or tissues. In certain embodiments, the indicator exhibits at least a 10-fold increase in bioluminescence intensity at wavelengths greater than 600 nm in response to physiological calcium concentrations, and may be encoded by a nucleic acid and expressed in a mammalian cell or in a transgenic animal.

In another aspect, the disclosure provides compositions for bioluminescence imaging comprising a polyethylene glycol (PEG)-conjugated luciferase substrate, wherein the substrate is a coelenterazine (CTZ) or diphenylterazine (DTZ) analog covalently linked to a PEG moiety. The PEGylated substrate exhibits increased water solubility relative to the non-PEGylated substrate and is suitable for in vivo administration to an animal for bioluminescence imaging with an ATP-independent luciferase. In certain embodiments, the PEG moiety has a molecular weight of at least 2,000 Da, and the PEGylated substrate is PEG10 k-DTZ. The PEGylated substrate may be deliverable in normal saline at a concentration of at least 20 mM, and may be hydrolyzable in vivo to release the active luciferin. Such compositions may be used for bioluminescence imaging of brain or liver tissue in a mammal, and may enable high-speed video-rate bioluminescence imaging in freely moving animals. In some embodiments, the luciferin substrate is caged by a caging group at the C3 position of an imidazopyrazinone core, and the PEGylation may affect bioluminescence signal kinetics and duration. The covalent conjugation may be via an ester, caronate, or amide linkage.

The disclosure also provides methods of non-invasive bioluminescence imaging in a mammal, comprising administering to the mammal a PEGylated luciferase substrate composition as described above, expressing an ATP-independent luciferase in a tissue of the mammal, and detecting bioluminescence emission from the tissue.

Further aspects of the invention include compounds comprising a coelenterazine (CTZ) or diphenylterazine (DTZ) analog covalently conjugated at the C3 carbonyl group to a cleavable substituent, wherein the substituent is selected from hydrophilic polymers, saccharides, amino acids, peptides, zwitterionic groups, or combinations thereof. The substituent enhances aqueous solubility, stability, pharmacokinetics, or in vivo distribution of the luciferin, and the conjugate is cleavable in vivo to release the luciferin. In certain embodiments, the substituent is a polyethylene glycol (PEG) moiety having a molecular weight between 2 kDa and 20 kDa.

The disclosure further provides methods for generating sensory luciferins, comprising derivatizing a luciferin molecule at the C2 position of the imidazopyrazinone core with a chemical moiety that comprises a sensory functionality. The derivatized luciferin is capable of being recognized and utilized by NanoLuc or a NanoLuc-derived luciferase to produce a detectable signal in response to the sensory functionality. In some embodiments, the derivatization at the luciferin C2 position is performed using click chemistry, such as a copper-catalyzed azide-alkyne cycloaddition (CuAAC) or a strain-promoted azide-alkyne cycloaddition (SPAAC), to conjugate the sensory chemical moiety to the luciferin. The sensory chemical moiety may be selected from metal ion sensors, pH sensors, redox-sensitive groups, and enzyme-responsive groups.

Accordingly, it is an object of the presently disclosed subject matter to provide engineered luciferases, modified luciferase substrates and related methods. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, can be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

Like numbers refer to like elements throughout. In the figures, the thickness of certain lines, layers, components, elements or features can be exaggerated for clarity. Where used, broken lines illustrate optional features or operations unless specified otherwise. For a more complete understanding of the presently disclosed subject matter, reference is now made to the below drawings.

FIG. 1 includes schematic illustration of the chemical structures of coelenterazine (CTZ), furimazine (FRZ), diphenylterazine (DTZ), pyCTZ (3a in Table 2), 8pyDTZ (3c in Table 2) and 6pyDTZ (3d in Table 2).

FIGS. 2A and 2B show chemiluminescence (FIG. 2A) and bioluminescence (FIG. 2B) spectra for synthetic CTZ and DTZ analogs described herein. Chemiluminescence was initiated with peroxymonocarbonate generated in situ. Bioluminescence was determined with 1 nM teLuc in PBS.

FIGS. 3A through 3D show the relative bioluminescence intensities of various luciferases. Total signals were integrated for the first 10 min after injection of indicated substrates (the final concentrations were 25 μM) in the presence of 1 nM purified (FIG. 3A) teLuc and (FIG. 3B) NanoLuc, or 10 nM (FIG. 3C) RLuc8 and (FIG. 3D) aequorin in PBS. Values were normalized to the intensity of CTZ in each group.

FIGS. 4A through 4D show results of efforts to engineer a LumiLuc luciferase. FIG. 4A is a schematic of a procedure to derive LumiLuc from teLuc. FIG. 4B is an illustration of the putative substrate-binding site and LumiLuc mutations from teLuc. CTZ is shown as spheres and mutated residues are presented in sticks. FIG. 4C shows the bioluminescence of pyCTZ, 6pyDTZ, or 8pyDTZ in the presence of either teLuc or LumiLuc. FIG. 4D shows normalized bioluminescence emission spectra of pyCTZ, 6pyDTZ, and 8pyDTZ in the presence of LumiLuc.

FIG. 5 shows alignments of primary polypeptide sequences of NanoLuc (SEQ ID NO. 1), teLuc (SEQ ID NO. 2), and LumiLuc (SEQ ID NO. 3). teLuc mutations are highlighted. Likewise, all LumiLuc mutations are highlighted.

FIGS. 6A through 6C show the comparison of luciferase-luciferin pairs in enzyme-based assays. FIG. 6A shows the determination of apparent Michaelis constants (KM) by substrate titrations (teLuc-DTZ: 9.9±0.9 μM; NanoLuc-FRZ: 9.1±0.6 μM; LumiLuc-8pyDTZ: 4.6±0.6μ; LumiLuc-pyCTZ: 13.1±0.8μ; LumiLuc-6pyDTZ: 11.0±1.2μ M). Final concentrations of all enzymes were 20 pM. Substrate concentrations varied from 0.78 to 50 μM, and peak bioluminescence intensities at individual substrate concentrations were used to fit the Michaelis-Menten equation. FIG. 6B shows the total bioluminescence over the first 10 min post addition of corresponding luciferins. Final concentrations for luciferins were 10 UM and final concentrations for luciferases were 50 pM. Data are normalized to the intensity of Akaluc-AkaLumine and shown as mean and s.d. from three independent measurements. Under this condition, LumiLuc-8pyDTZ produced about 1200-fold higher photon flux than Akaluc-AkaLumine. Assays in panels a and b were all performed in PBS. FIG. 6C shows the bioluminescence kinetic of LumiLuc-8pyDTZ in PBS or in a formulated assay buffer containing 1 mM CDTA, 0.5% Tergitol NP-40, 0.05% Antifoam 204, 150 mM KCl, 100 mM MES, pH 6.0, 1 mM DTT, and 35 mM thiourea.

FIG. 7 shows the comparison of luciferase-luciferin pairs in live mammalian cells. Measurements were performed with 5000 HEK 293T cells in PBS. Luciferase genes were introduced by transient transfection (about 70% transfection efficiency). Final concentrations for FRZ, DTZ, and 8pyDTZ were 20 μM, and final concentrations for AkaLumine and D-luciferin were 100 μM. Signals were integrated over the first 10 min post injection of substrates. Data are presented as mean and s.d. from three independent measurements. Under this condition, LumiLuc-8pyDTZ produced about 300-fold higher photon flux than Akaluc-AkaLumine.

FIGS. 8A and 8B show the bioluminescence of teLuc- and LumiLuc-expressing HEK 293T cells. Images were acquired (FIG. 8A) without or (FIG. 8B) with a 600-700 nm bandpass filter. Values for relative brightness were normalized to teLuc in the presence of 6.25 μM DTZ.

FIGS. 9A and 9B show bioluminescence characterizations of Hela cells stably expressing luciferases. (FIG. 9A) Bioluminescence intensities integrated over the first 10 mins post injection of substrates. The y-axis is shown in a logarithmic scale. Under this condition, LumiLuc-8pyDTZ produced about 190-fold higher photon flux than Akaluc-AkaLumine. (FIG. 9B) Decay kinetics. Assays were performed with 20 μM substrates and 500 HeLa cells. Data are presented as mean and s.d. from three independent measurements.

FIGS. 10A and 10B show the tracking of tumor growth in a xenograft mouse model with various luciferase-luciferin pairs. (FIG. 10A) BLI (n=4) on day 1, 3, 5, and day 7. 10\hat{ }4 luciferase-expressing Hela cells were injected to the left and right dorsolateral trapezius regions and 10\hat{ }5 cells were injected to the left and right dorsolateral thoracolumbar regions of NU/J mice. For i.v. administration of substrates, AkaLumine-HCl (3 μmol/mouse) and 8pyDTZ (0.2 μmol/mouse) were dissolved in normal saline, and DTZ (0.3 μmol/mouse) was formulated with a mixture of organic cosolvents. (FIG. 10B) Comparison of luciferase-luciferin pairs at tumor sites inoculated with 10\hat{ }4 cells. (*p<0.05 for LumiLuc-8pyDTZ and teLuc-DTZ, and for LumiLuc-8pyDTZ and Akaluc-AkaLumine; **p<0.05 for LumiLuc-8pyDTZ and Antares2-DTZ).

FIGS. 11A-11C show tracking of tumor growth in a xenograft mouse model with various luciferase-luciferin pairs. (FIG. 11A) Hela cells stably expressing indicated luciferase were injected at four sites of each female NU/J mouse. 10\hat{ }4 cells were injected to the left and right dorsolateral trapezius regions and 10\hat{ }5 cells were injected to the left and right dorsolateral thoracolumbar regions. BLI were obtained on days 1, 3, 5, 7, 14, and 28 (n=4). Pseudocolored images are presented on a scale of 5×10\hat{ }5 to 5×10\hat{ }7 p/sec/cm\hat{ }2/sr. Images are identical to FIG. 4, except for that data on days 14 and 28 are presented here and that a different scale is used for pseudocoloring. (FIG. 11B) Comparison of luciferase-luciferin pairs at tumor sites inoculated with 10\hat{ }5 cells (*p<0.05 for LumiLuc-8pyDTZ and Akaluc-AkaLumine; **p<0.05 for LumiLuc-8pyDTZ and teLuc-DTZ; ***p<0.05 for LumiLuc-8pyDTZ and teLuc-8pyDTZ). (FIG. 11C) Bioluminescence decay kinetics for i.v.-injected luciferins (1.5 μmol for AkaLumine, 0.2 μmol for 8pyDTZ, and 0.3 μmol for DTZ) in a xenograft NU/J mouse model. Measurements were done 5 days after tumor implantation. Quantifications were made at sites initially inoculated with 10\hat{ }5 cells. Data are presented as mean and s.d. from four replicates.

FIGS. 12A-12F show the engineering and characterization of BRET-based LumiScarlet and teScarlet. (FIG. 12A) Libraries screened for high BRET. Each X represents an amino acid residue randomized with the NNK codon, in which N=A/C/G/T and K=G/T. (FIG. 12B) Bioluminescence spectra of constructs selected from each library in the presence of 8pyDTZ. (FIG. 12C) Normalized fluorescence excitation and emission spectra of mScarlet-I and bioluminescence emission of LumiLuc-8pyDTZ, showing excellent spectral overlap for BRET. (FIG. 12D) Comparison of LumiLuc and LumiScarlet (100 pM purified enzymes) for bioluminescence integrated over the first 10 min post injection of 20 μM 8pyDTZ. (FIG. 12E) Schematic diagram of teScarlet, a genetic fusion of mScarlet-I and teLuc. (FIG. 12F) Bioluminescence emission of teScarlet in the presence of DTZ, showing significant emission longer than 600 nm.

FIGS. 13A-13D show BRET-based LumiScarlet for deep tissue BLI. (FIG. 13A) Schematic diagram of LumiScarlet, a genetic fusion of mScarlet-I and LumiLuc. (FIG. 13B) Bioluminescence emission of LumiScarlet in the presence of pyCTZ, 6pyDTZ, or 8pyDTZ, showing significant emission longer than 600 nm. (FIG. 13C) Comparison of LumiLuc-8pyDTZ, LumiScarlet-8pyDTZ, and Akaluc-AkaLumine in NU/J mice (n=4) at 4 h post i.v. injection of 10\hat{ }6 luciferase-expressing HeLa cells. (FIG. 13D) Quantitative analysis of bioluminescence from the regions around the lungs in panel c (n.s.: not significant).

FIGS. 14A-14C show the chemical structures and maximum bioluminescence (BL) emission wavelength of (FIG. 14A) pyOMeCTZ, (FIG. 14B) pyDTZ, and (FIG. 14C) AkaLumine in the presence of its corresponding luciferase.

FIGS. 15A-15C show engineering of OpyLuc luciferase for pyDTZ selectivity over pyOMeCTZ. (FIG. 15A) The schematic representation of directed evolution to derive OpyLuc. (FIG. 15B) Illustration of the putative substrate-binding site and OpyLuc mutations. CTZ is shown as spheres and mutated residues near the binding site are highlighted in yellow. (FIG. 15C) Normalized bioluminescence activity ratio of pyCTZ/pyOMeCTZ in the presence of either teLuc or Opy Luc.

FIG. 16 shows the alignments of primary polypeptide sequences of Opy Luc (SEQ ID NO. 6) and teLuc (SEQ ID NO. 2). Opy Luc mutations are highlighted.

FIGS. 17A and B show the results of the analysis of orthogonal luciferase-luciferin pairs. (FIG. 17A) Schematic representation of spectral-resolved and orthogonal luciferase-luciferin pairs. (FIG. 17B) Normalized bioluminescence emission spectra of RLuc8-pyOMeCTZ (purple), OpyLuc-pyDTZ (green), and Akaluc-AkaLumine pairs (red).

FIGS. 18A and 8B show the determination of apparent Michaelis constants (KM) by substrate titrations. (FIG. 18A) OpyLuc-pyDTZ: 9.8±1.7 μM. (FIG. 18B) RLuc8-pyOMeCTZ: 6.3±0.5 μM. Final concentrations of all enzymes were 100 pM. Substrate concentrations varied from 0.78 to 50 μM, and 10 min integration of total bioluminescence intensities at individual substrate concentrations were used to fit the Michaelis-Menten equation.

FIGS. 19A-19D show bioluminescence imaging of (FIGS. 19A-B) purified recombinant RLuc8, OpyLuc, and Akaluc and (FIGS. 19C-D) luciferase-expressing HEK 293T cells. (FIG. 19A) Images were acquired without a filter or with either a 360-500 nm, 495-580 nm, or 600-700 nm bandpass filter. (FIG. 19B) Quantitative values for each tested luciferase-luciferin pair. Final concentrations were 10 nM for RLuc8 and Opy Luc; 100 nM for Akaluc; 30 μM corresponding luciferin. (FIG. 19C) Live HEK293T cells were transfected with either RLuc8, Akaluc, or OpyLuc. 5000 cells per well for RLuc8 and OpyLuc; 30,000 cells per well for Akaluc. Images were acquired without a filter after addition of 1:10 μM pyDTZ, 2:100 μM AkaLumine, or 3:25 μM pyOMeCTZ. (FIG. 19D) Quantitative analysis of BL signals gained from (FIG. 19C).

FIG. 20A shows results of the triple luciferase assay in live HEK293T after co-transfection of SRE-RLuc8, ARE-OpyLuc, and NFKB-Akaluc plasmids. FIG. 20B shows the BL signals from each luciferase were acquired from intact cells after adding its corresponding luciferin. The cells were induced by 1:10 ng/ml TNFa; 2: 20% FBS; 3:50 μM tBHQ; 4: 20% FBS+50 μM tBHQ; 5: 20% FBS+10 ng/ml TNFa, 6:50 μM tBHQ+10 ng/ml TNFa; 7: 20% FBS+50 μM tBHQ+10 ng/ml TNFa for 16 h post PEI transfection.

FIGS. 21A-21I show results of triple luciferase assay in live HEK293T after co-transfection of SRE-RLuc8, ARE-OpyLuc, and NFKB-Akaluc plasmids. (FIG. 21A) Schematic of triple luciferase assay in live HEK293T after co-transfection of SRE-RLuc8, ARE-OpyLuc, and NFKB-Akaluc plasmids. The emission spectra were acquired from intact cells after adding Optimal Mix solution. The cells were (FIG. 21B) non-treated, induced by (FIG. 21C) 20% FBS, (FIG. 21D) 50 μM tBHQ, (FIG. 21E) 10 ng/mL TNFa, (FIG. 21F) 20% FBS+10 ng/ml TNFa, (FIG. 21G) 20% FBS+50 μM tBHQ, (FIG. 21H) 50 μM tBHQ+10 ng/ml TNFa, (FIG. 21I) 20% FBS+50 μM tBHQ 10 ng/ml+TNFa for 16 h post PEI transfection.

FIGS. 22A-22C show bioluminescent Ca²⁺ biosensors. (FIG. 22A) Schematic diagrams of LumiCameleon1 and LumiCameleon2. (FIG. 22B) Bioluminescence emission of LumiCameleon1 in the presence of pyCTZ, showing large dynamic range in response to Ca²⁺. (FIG. 22C) Bioluminescence emission of LumiCameleon2 in the presence of DTZ.

FIG. 23 presents a mechanistic comparison of this disclosure with other common bioluminescent indicators of in vivo imaging importance. (a) Reaction-based bioluminescent indicators: a caged luciferin is utilized, where a specific activity can remove the caging group and activate the luciferin, allowing for the specific detection of bioactivity. (b) Sensory luciferase-based bioluminescent indicators: a luciferase is engineered to be responsive to a specific analyte by strategically inserting and fusing a sensory domain to the luciferase. (c) Sensory luciferin-based bioluminescent indicators (this work): an analyte-binding moiety (e.g., a K-binding crown ether) is strategically introduced to the luciferin, leading to the analyte-responsive modulating of the bioluminescence reaction. CG, caging group; BL, bioluminescence.

FIG. 24 presents the design of potassiorin and initial evaluation with BREP luciferase. (a) Illustration of the DTZ structure and the installation of a K-binding crown ether ring to derive potassiorin. The C2, C6, and C8 derivatizations on DTZ are highlighted. (b) Schematic illustration of the domain arrangements of BREP, a fusion of mScarlet-I and teLuc through a three amino acid linker. (c) Illustration of a modeled structure of NanoLuc (cyan ribbon) in complex with CTZ (magenta sticks). The C2, C6, and C8 derivatizations on CTZ are highlighted. (d,e) Bioluminescence emission spectra of BREP in the presence of potassiorin (d) or DTZ (e) with or without 150 mM KCl. Presented are the averages from three technical replicates. (f) Bioluminescence intensities of BREP and potassiorin at 590 nm in the presence of the indicated concentrations of K⁺ or Na⁺. n=3 technical replicates. A one-site binding model was used to fit the data and derive the apparent dissociation constants (K_{d}). BL, bioluminescence.

FIG. 25. Presents the Synthetic route to chemically synthesize potassiorin. (a) Preparation of 3-(4-(2-azidoethoxy)phenyl)-1,1-diethoxypropan-2-one (6) from 4-benzyloxybenzyl alcohol (1). (b) Preparation of potassiorin from N-phenyldiethanolamine (7) and 6.

FIG. 26. Presents the engineering of BRIPO and illustration of key mutations. (a) Flowchart for the protein engineering process with mutations and dynamic ranges (determined with cell lysates) highlighted. (b) Structure of NanoLuc bound with an inactive 3-methoxy-furimazine luciferin analog (Protein Data Bank entry 7SNT). Residues in the luciferase domain that are mutated in BRIPO are also indicated. BL, bioluminescence.

FIG. 27 illustrates the in vitro characterization of BRIPO. (a) Schematic illustration of BRIPO with mutations from BREP highlighted. (b,c) Bioluminescence emission spectra of BRIPO in the presence of potassiorin (b) or DTZ (c) with or without 150 mM KCl. Presented are the averages from three technical replicates. (d) Bioluminescence spectra of BRIPO and potassiorin with the indicated concentrations of KCl. Presented are the averages from three technical replicates. (e) Bioluminescence intensities of BRIPO and potassiorin at 590 nm in the presence of the indicated concentrations of K⁺ or Na⁺. n=3 technical replicates. A one-site binding model was used to fit the data and derive the apparent dissociation constants (K_{d}). (f) Normalized bioluminescence intensity of BRIPO and potassiorin in the presence of different metal ions: Na (15 mM), Zn²⁺ (10 μM), Ca²⁺ (2 mM), Fe²⁺ (10 M), Cu²⁺ (100 nM), Mn²⁺ (10 μM), K⁺ (150 mM), Mg²⁺ (2 mM). n=3 technical replicates. BL, bioluminescence.

FIG. 28 presents the sequence alignment of BREP (SEQ ID NO: 46), BRIPO0.5 (SEQ ID NO: 47), and BRIPO (SEQ ID NO: 48). Sequences derived from mScarlet-I and teLuc are colored in red and cyan, respectively. Linker residues are colored in gray. Mutations gained during protein engineering are shaded in yellow (from random mutagenesis) and orange (from site-directed randomization).

FIG. 29 presents Additional in vitro characterization of BRIPO. (a) Bioluminescence responses of BRIPO in the presence of potassiorin (left) or DTZ (right) to 150 mM K⁺ and the indicated concentrations of Cu²⁺. n=3 technical repeats. (b) Potassiorin concentration dependency of BRIPO bioluminescence at 590 nm in the presence or absence of 150 mM KCl. n=3 technical repeats. The Michaelis-Menten equation was used to fit the data and derive the Michaelis constants (KM). BL, bioluminescence.

FIG. 30 demonstrates the imaging K⁺ efflux in cultured cell lines. (a) Schematic illustration of nigericin-mediated K⁺ efflux in HEK 293T cells in a low K⁺ buffer. (b) Representative pseudo-colored bioluminescence images of BRIPO-expressing HEK 293T cells in the presence of potassiorin (top) or DTZ (bottom) before (left) and after (right) treatment with a combination of nigericin, ouabain, and bumetanide. Scale bar: 50 μm. (c) Quantification of bioluminescence intensity changes of individual cells from experiments in panel b. Data are presented as mean±s.d. (n=39 cells for the potassiorin group, n=57 cells for the DTZ group). (d) Schematic illustration of a stable HEK 293T cell line treated with arachidonic acid to open the mTrek channel and induce K⁺ efflux. (e) Representative pseudocolored bioluminescence images of BRIPO-expressing HEK 293T cells stably expressing mTrek and other ion channels in the presence of potassiorin (top) or DTZ (bottom) before (left) and after (right) treatment with arachidonic acid. Scale bar: 50 μm. (f) Quantification of bioluminescence intensity changes of individual cells from experiments in panel e. Data are presented as mean±s.d. (n=33 cells for the potassiorin group, n=42 cells for the DTZ group). In panels c and f, the baselines were corrected using a monoexponential decay model, and the P value was derived from unpaired two-tailed t-tests. The GraphPad Prism software does not provide extract P values below 10-15. This figure is created with BioRender.com. BL, bioluminescence. Arb. units, arbitrary units.

FIG. 31 presents the Imaging K⁺ influx into HEK 293T cells in high K⁺ buffer. (a) Representative pseudo-colored bioluminescence images of BRIPO-expressing HEK 293T cells in a high K⁺ (200 mM) buffer in the presence of potassiorin (top) or DTZ (bottom) before (left) and after (right) treatment with a combination of nigericin, ouabain, and bumetanide. Scale bar: 50 μm. (b) Quantification of bioluminescence intensity changes of individual cells from experiments in panel a. Data are presented as mean #s.d. (n=85 cells for the potassiorin group, n=85 cells for the DTZ group).

FIG. 32 presents the imaging K⁺ efflux in primary mouse neurons and the brains of live mice. (a) Representative fluorescence and pseudo-colored bioluminescence images of BRIPO-expressing primary mouse neurons. Glutamate was used to induce potassium efflux. Scale bar, 50 μm. (b) Quantification of bioluminescence intensity changes of individual responsive neurons upon glutamate treatment. Data are presented as mean±s.d. (n=25 cells for the potassiorin group, n=31 cells for the DTZ group). (c) Schematic illustration of stereotactic intracranial administration of AAVs containing the BRIPO gene and other general experiment procedures. (d) Quantification of bioluminescence intensity changes of individual animals. Data are presented as mean±s.e.m. (n=5 mice for the potassiorin group, n=6 mice for the DTZ group). In panels b and d, the baselines were corrected using a monoexponential decay model. and the P value was derived from unpaired two-tailed t-tests. The GraphPad Prism software does not provide extract P values below 10⁻¹⁵. This figure is created with BioRender.com.

FIG. 33 presents the characterization of R15-DTZ for Na⁺ sensing. (a) Bioluminescence emission spectra of BREP in the presence of R15-DTZ with or without 150 mM NaCl. Presented are the averages from three technical replicates. (b) Bioluminescence intensities of BREP and R15-DTZ at 590 nm in the presence of the indicated concentrations of K⁺ or Na⁺. n=3 technical replicates. A one-site binding model was used to fit the data and derive the apparent dissociation constants (K_{d}). BL, bioluminescence.

FIG. 34 presents the 1H-NMR (a) and 13C-NMR (b) spectra for compound 11.

FIG. 35 presents the 1H-NMR (a) and 13C-NMR (b) spectra for potassiorin (compound 13).

FIG. 36 presents an SDS-PAGE showing the purity of the prepared BREP and BRIPO proteins.

FIG. 37 presents the synthetic route to chemically prepare R15-DTZ.

FIG. 38 presents the engineering and in vitro characterization of eBRIC. (A) Flowchart showing the engineering of eBRIC from BRIC. The ratios of bioluminescence in the presence of 39 μM Ca²⁺ to the absence of Ca²⁺ are also included, as determined from bacterial lysate-based assays. (B) Domain arrangement of eBRIC with five mutations from BRIC highlighted as magenta balls. The CaM (calmodulin), M13, teLuc, and mScarlet-I domains are colored in marine blue, golden yellow, pale cyan, and salmon pink, respectively. (C) Highlight of mutations on CaM and M13. ColabFold v1.5.5 was used to generate model structures for BRIC and eBRIC. The CaM portions in BRIC and eBRIC are colored blue and cyan, respectively, while the M13 portions in BRIC and eBRIC are colored yellow and light orange, respectively. D474 and N515 in BRIC are shown in orange, while E474 and D515 in eBRIC are shown in magenta. (D) Bioluminescence spectra of eBRIC in the presence of DTZ and varying concentrations of free Ca²⁺. (E) Ca²⁺-dependency of eBRIC bioluminescence at 600 nm (n=3 technical replicates). A four-parameter Hill equation was used to fit the data to determine the dissociation constant (K_{d}=2.3 μM and Hill coefficient=1.3).

FIG. 39 illustrates the imaging Ca²⁺: dynamics in cultured mammalian cell lines using eBRIC (A) Representative pseudocolored bioluminescence images of histamine-induced Ca²⁺ dynamics in Hela cells. Scale bar, 20 μm. (B) Left: Intensity traces for individual cells. The color of the traces corresponds to the colors of the arrows used to highlight individual cells in the left images. The baselines of the traces were corrected for monoexponential decay resulting from substrate consumption. Dashed lines indicate the four time points corresponding to the images shown in panel A, and the timing of histamine addition is marked by the gray line. Right: Parallel comparison between eBRIC and BRIC in terms of the maximal changes (ΔBL/BL₀) of individual cells induced with histamine. Data are expressed as mean±s.d. (n=33 cells for eBRIC and 34 cells for BRIC), with the P value derived from unpaired two-tailed t-tests. (C) Left: Representative pseudocolored bioluminescence images of eBRIC-expressing HEK 293T cells before and after acetylcholine stimulation. Right: Intensity traces for individual cells. Data are expressed as mean±s.d. (n=20 cells).

FIG. 40 presents a comparison of BRIC and eBRIC for imaging Ca²⁺: influx during the depolarization of cultured primary mouse neurons. (A) Representative fluorescence and bioluminescence images of primary mouse neurons expressing eBRIC or BRIC in response to high K (30 mM) depolarization. Scale bar, 50 μm. (B) Left: Quantification of the responses of eBRIC- or BRIC-expressing neurons. Right: Comparison of the maximal changes (A BL/BL₀) of individual cells. Data are expressed as mean±s.d. (n=24 cells for eBRIC and 17 for BRIC), with the P value determined through unpaired two-tailed t-tests.

FIG. 41. Imaging Ca²⁺ dynamics in head-fixed awake mice. (A) Scheme of BLI of head-fixed awake mice. eBRIC expression in the basolateral amygdala (BLA) region was achieved using AAV vectors. Prior to imaging, sDTZ was administered to mice via the tail vein. Footshocks were then employed as a means of stimulation. Images were acquired with a 100-ms exposure time without any intervals. (B) Representative bioluminescence intensity traces for mice expressing either eBRIC or BREP (negative control) following 13 consecutive footshock stimulation trials. (C) Left: Quantification of intensity changes of eBRIC- and BREP-expressing mice in response to footshock. Data is presented as mean±s.d. and based on images of 4 mice for eBRIC and 3 mice for BREP, each consisting of 13 trials. Right: Comparison of the eBRIC and BREP groups by averaging the responses of individual mice over 13 trials. Data are expressed as mean±s.d. (n=4 mice for eBRIC and 3 mice for BREP), with the P value derived from unpaired two-tailed t-tests. This figure is created with BioRender.com.

FIG. 42. Synthesis of water-soluble DTZ derivatives and evaluation of solubility, stability, and sensitivity for in vivo bioluminescence brain imaging. (A) Synthetic route to modify DTZ with three lengths of polyethylene glycol (PEG) chains (PEG 2 k, 5 k, or 10 k) via the C3 carbonyl group of DTZ. (B) Photos of PEGylated DTZ variants dissolved in normal saline at the indicated near-saturation concentrations. (C) Representative bioluminescence images of live mice with hippocampal BREP luciferase expression upon the tail vein delivery of the luciferins at the indicated dose and buffer conditions. Images with peak bioluminescence intensities were presented in pseudocolor overlaid on corresponding brightfield images. (D) Quantification of bioluminescence intensity over time for each group in panel C. (E) Comparison of peak bioluminescence intensity (top) and bioluminescence integrated over 15 min (bottom). The numbered conditions refer to the groups presented in panel D. P values were determined by ordinary one-way ANOVA followed by Dunnett's multiple comparisons test. Data are presented as mean±s.e.m. (in panels C-E, n=4 mice for 5 mM sDTZ, n=5 mice for 15 and 25 mM sDTZ, and n=3 mice for other groups).

FIG. 43. Evaluation of sDTZ paired with BREP for in vivo bioluminescence liver imaging. (A) Representative bioluminescence images of live mice with liver BREP luciferase expression upon the tail vein delivery of the luciferins at the indicated dose and buffer conditions. Images with peak bioluminescence intensities were presented in pseudocolor overlaid on corresponding bright-field images. (B) Quantification of bioluminescence intensity over time for each group in panel A. (C) Comparison of peak bioluminescence intensity (top) and bioluminescence integrated over 15 min (bottom). The numbered conditions refer to the groups presented in panel B. P values were determined by ordinary one-way ANOVA followed by Tukey's multiple comparison tests. Data are presented as mean±s.e.m. (n=3 mice for each group).

FIG. 44. Evaluation of water-soluble Fz and CFz variants, sFz and sCFz, paired with Antares for in vivo bioluminescence brain imaging. (A) Chemical structures of sFz and sCFz. PEG10 k was used to cage the substrates. Photos of individual substances (25 mM) dissolved in normal saline are also shown. (B) Representative bioluminescence images of live mice with hippocampal Antares luciferase expression upon the tail vein delivery of the luciferins at the indicated dose and buffer conditions. Images with peak bioluminescence intensities were presented in pseudocolor overlaid on corresponding brightfield images. (C) Quantification of bioluminescence intensity over time for mice in the Fz and sFz groups. (D) Quantification of bioluminescence intensity over time for mice in the CFz and sCFz groups. (E) Comparison of peak bioluminescence intensity (left) and bioluminescence integrated over time (right) for the Fz and sFz groups. (F) Comparison of peak bioluminescence intensity (left) and bioluminescence integrated over time (right) for the CFz and sCFz groups. In panels E and F, the numbered conditions refer to the groups presented in panels C and D; P values were determined by ordinary one-way ANOVA followed by Dunnett's multiple comparisons test. Data are presented as mean±s.e.m. (in panels B-F, n=4 mice for CFz in HPCD/PEG and sCFz groups, and n=3 mice for other groups).

FIG. 45. Further brightness comparison and high-frame video-rate in vivo bioluminescence brain imaging using BREP and sDTZ. (A) Representative bioluminescence images of live mice with hippocampal luciferase expression upon the tail vein delivery of the indicated luciferins at the indicated dose using normal saline. Comparisons were made between BREP (paired with sDTZ or sCFz) and Akaluc (paired with Akalumine). Images with peak bioluminescence intensities were presented in pseudocolor overlaid on corresponding brightfield images. (B) Quantification of bioluminescence intensity over time among different groups. (C) Comparison of peak bioluminescence intensity (top) and bioluminescence integrated over time (bottom). The numbered conditions refer to the groups presented in panel B. P values were determined by ordinary one-way ANOVA followed by Tukey's multiple comparison tests. Data are presented as mean values±s.e.m. (n=3 mice for BREP and 10 mM sDTZ, n=4 mice for Akaluc and 10 or 30 mM AkaLumine, and n=5 mice for all other groups). The same data for BREP and sDTZ in panels A-C are also presented in FIG. 1C-E. (D) High frame-rate imaging of a mouse with hippocampal BREP luciferase expression. The mouse was injected with sDTZ via the tail vein right before imaging. Brightfield and bioluminescent images (15-ms exposure time for each) were alternately acquired using an EMCCD camera. The first frame (at t=0 min) was shown. A series of 280 frames of bioluminescent images at each time point (t=0, 3, 6, 12 and 18 min) were acquired, and the total 1,400 frames were combined to create the summation image.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Disclosed are methods of generating bioluminescent indicators. In embodiments, there is disclosed a method of modifying luciferins with sensory moieties as seen in FIG. 23, c. In particular embodiments there is disclosed the design and synthesis of an artificial luciferin which selectively binds to potassium (K⁺) ions. The artificial luciferin is named potassiorin. Still other embodiments present the engineering of an artificial luciferase named BRIPO (Bioluminescent Red Indicator for Potassium) to work in conjunction with potassiorin, producing bioluminescence signals responsive to the physiological concentrations of K⁺. As illustrated in the below-described tests and examples, the results of practicing embodiments and methods disclosed demonstrate a capability of real-time monitoring of K⁺ dynamics. The methods and embodiments disclosed herein advantageously provide a powerful approach for the design of novel bioluminescent indicators.

### I. General Considerations

Provided herein are chemically modified luciferase substrates, namely coelenterazine (CTZ) and diphenylterazine (DTZ), for spectrally shifted emission and enhanced water solubility. Concurrently, teLuc was engineered into a LumiLuc luciferase, which is highly active toward the new modified substrates for intense blue, teal, and yellow emission. Moreover, provided herein is a new reporter, LumiScarlet, with significant emission longer than 600 nm. The disclosed multipronged approach yielded a new family of ATP-independent bioluminescent reporters, which have improved biochemical and photophysical properties and are expected to have broad applications.

To elaborate, a series of pyridyl CTZ and DTZ analogs, or luciferin compounds, with diverse emission profiles were prepared. The water solubility of these synthetic analogs generally increased by about 10-fold from their ancestors. Surprisingly, these substrate analogs can not only be paired with the new luciferases engineered herein, but also existing ATP-independent reporters, such as RLuc and aequorin.

Such luciferin compounds, as described further herein, can include the following structure:

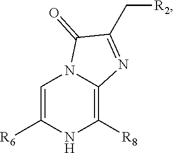

wherein R6 is selected from

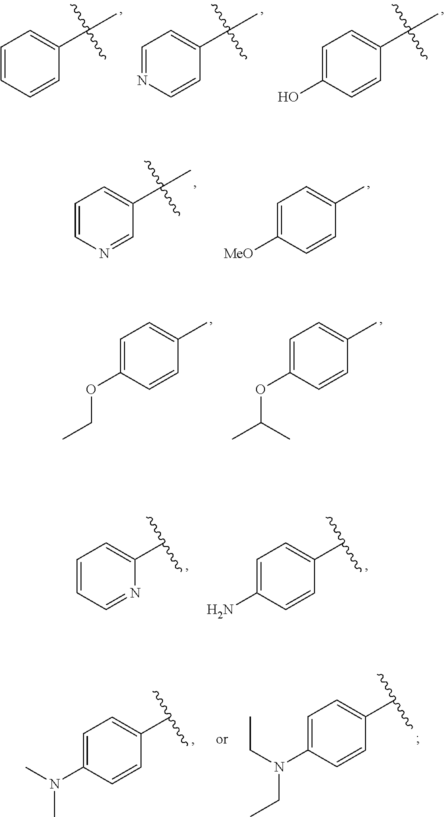

wherein R8 is selected from

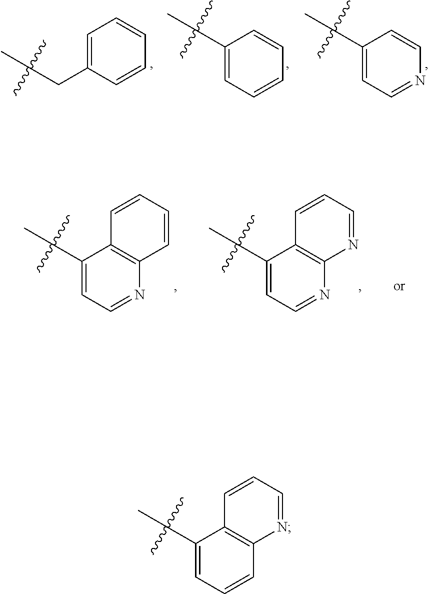 [SMILES: *Cc1ccccc1 ; *c1ccccc1 ; *c1ccncc1 ; *c1ccnc2ccccc12 ; *c1ccnc2ncccc12 ; *c1cccc2ncccc12]

and wherein R2 is selected from

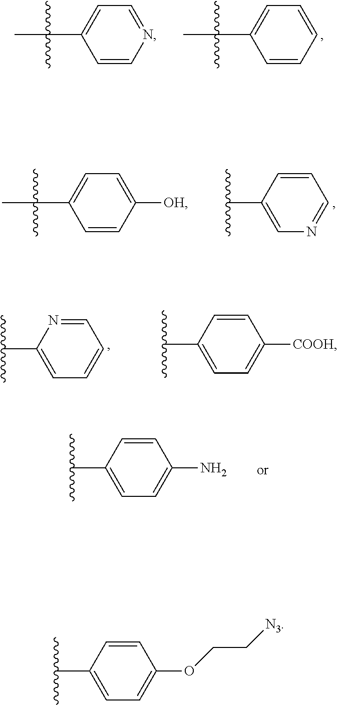

More particularly, the luciferin compounds include:

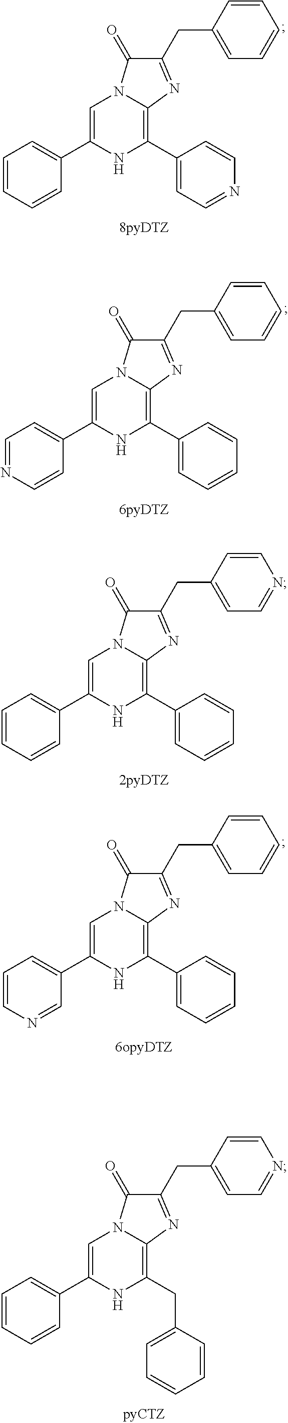 [SMILES: O=c1c(Cc2ccccc2)nc2c(-c3ccncc3)[nH]c(-c3ccccc3)cn1-2 ; O=c1c(Cc2ccccc2)nc2c(-c3ccccc3)[nH]c(-c3ccncc3)cn1-2 ; O=c1c(Cc2ccncc2)nc2c(-c3ccccc3)[nH]c(-c3ccccc3)cn1-2 ; O=c1c(Cc2ccccc2)nc2c(-c3ccccc3)[nH]c(-c3cccnc3)cn1-2 ; O=c1c(Cc2ccncc2)nc2c(Cc3ccccc3)[nH]c(-c3ccccc3)cn1-2]

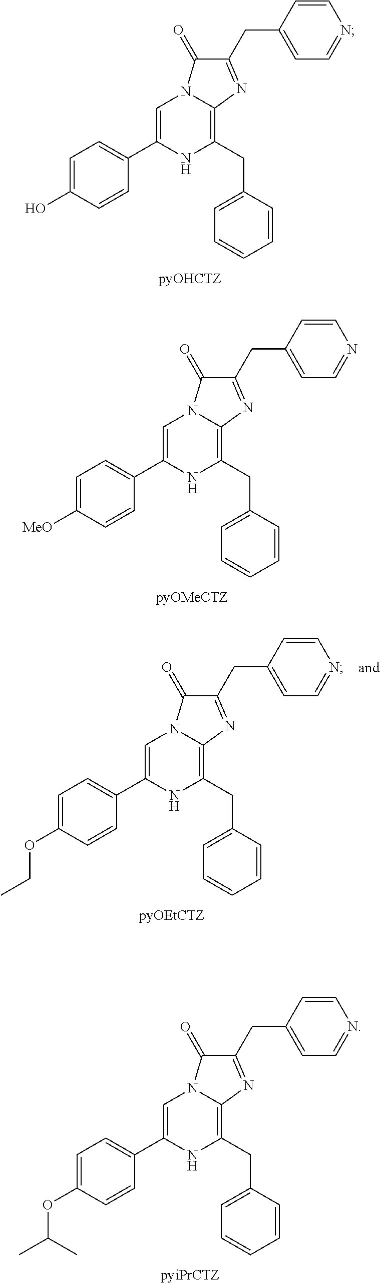 [SMILES: O=c1c(Cc2ccncc2)nc2c(Cc3ccccc3)[nH]c(-c3ccc(O)cc3)cn1-2 ; COc1ccc(-c2cn3c(=O)c(Cc4ccncc4)nc-3c(Cc3ccccc3)[nH]2)cc1 ; CCOc1ccc(-c2cn3c(=O)c(Cc4ccncc4)nc-3c(Cc3ccccc3)[nH]2)cc1 ; CC(C)Oc1ccc(-c2cn3c(=O)c(Cc4ccncc4)nc-3c(Cc3ccccc3)[nH]2)cc1]

Methods of making luciferin compounds are provided herein, and comprise making one or more pyridyl isomer substitutions at a C-2, C-6 and C-8 position of an imidazopyrazinone core according to the following chemical synthetic route:

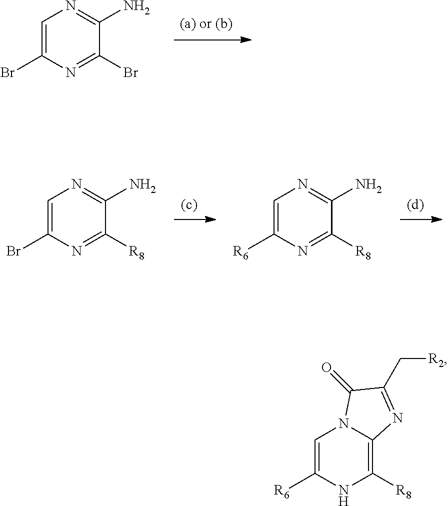

wherein step (a) comprises Suzuki coupling, comprising Pd (PPh₃)₄, Na₂CO₃, R₈-B(OH)₂, and/or EtOH; step (b) comprises Negishi coupling, comprising PhCH₂MgCl, ZnCl₂, (PPh₃)₂PdCl₂, and/or THF; step (c) comprises Suzuki coupling, comprising XPhos-Pd-G2, Na₂CO₃, R₆-B(OH)₂, and/or EtOH; and step (d) comprises acid-catalyzed ring closing, comprising corresponding α-ketoacetal, HCl, and/or dioxane.

Disclosed herein are luciferin compounds consisting of pyOMeCTZ, pyDTZ and AkaLumine, which comprise the following chemical structures:

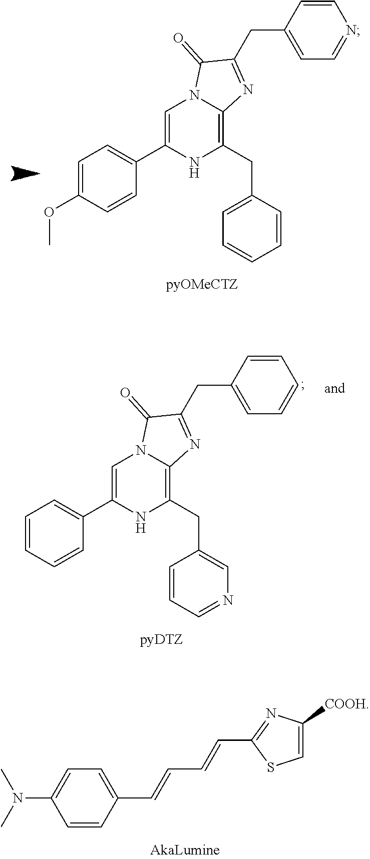

Moreover, the LumiLuc luciferase provided herein can in some embodiments comprise a substituted teLuc luciferase, including up to twelve substitutions, as discussed further hereinbelow. In some aspects, a fluorescent protein can be connected to the substituted luciferase polypeptides so as to allow bioluminescence resonant energy transfer (BRET) between the substituted luciferase polypeptide and the fluorescent protein.

Engineered luciferases provided herein, also referred to as bioluminescent proteins, include LumiLuc (SEQ ID NO. 3), LumiScarlet (SEQ ID NOs. 4 and 5), OpyLuc (SEQ ID NO. 6), teScarlet (SEQ ID NOs. 7 and 8), LumiCameleon1 (SEQ ID NOs. 9 and 10), and LumiCameleon2 (SEQ ID NOs. 11 and 12). Additional luciferases are also provided as follows: NanoLuc (SEQ ID NO. 1), teLuc (SEQ ID NO. 2), RLuc8 (SEQ ID NOs. 13 and 14), Akaluc (SEQ ID NOs. 15 and 16), NanoKAZ (SEQ ID NO. 17), and yeLuc (SEQ ID NO. 18).

In addition, provided herein are engineered mutually orthogonal luciferase-luciferin pairs for multiplexed cell-based bioluminescence (BL) assays. Disclosed triple-color BL systems feature the selectivity of synthetic substrates and production of well separated emission spectra from about 400 nm to about 650 nm. The disclosed spectral-resolved triple-color BL systems provide flexible and convenient approaches to monitor multiple biological events in either qualitative or quantitative manners.

New bioluminescent Ca²⁺ biosensors were also developed based on the modified luciferase compounds disclosed herein. These bioluminescent Ca²⁺ biosensors showed large bioluminescence increase in response to Ca²⁺, making them well suited for in vivo and in vitro applications.

The disclosed luciferase-luciferin pairs can also be used for in vivo monitoring of tumor models. For example, bioluminescence can be monitored in a tumor model by administering to that model, or otherwise establishing in the model, a luciferase expressing cell (e.g. a tumor cell expressing LumiLuc, teLuc, RLuc8 and OpyLuc), and administering to the model a luciferin (e.g. pyCTZ, pyOHCTZ, pyOMeCTZ, pyOEtCTZ, pyiPrCTZ, 2pyDTZ, 6pyDTZ, 60pyDTZ or 8pyDTZ). Using these luciferase-luciferin pairs as reporter systems the bioluminescence can be used as a tool to monitor the tumor.

The present disclosure provides for the use of the new luciferase-luciferin pairs for assays of cell signaling pathways. By way of example and not limitation, the disclosed luciferase-luciferin pairs can allow for the monitoring of particular cell signaling pathways, which can be implemented for candidate compound and drug screening applications.

### II. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose, etc., is meant to encompass in some embodiments variations of ±20%, in some embodiments ±10%, in some embodiments ±5%, in some embodiments ±1%, in some embodiments ±0.5%, and in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the term “subject” refers to an individual (e.g., human, animal, or other organism) to be treated by the methods or compositions of the present disclosure. Subjects include, but are not limited to, mammals (e.g., murines, simians, equines, bovines, porcines, canines, felines, and the like), and includes humans. In the context of the disclosure, the term “subject” generally refers to an individual who will receive or who has received treatment for a condition characterized by the presence of bacteria (e.g., Bacillus anthracis (e.g., in any stage of its growth cycle), or in anticipation of possible exposure to bacteria. As used herein, the terms “subject” and “patient” are used interchangeably, unless otherwise noted.

As used herein, the terms “effective amount” and “therapeutically effective amount” are used interchangeably and refer to the amount that provides a therapeutic effect, e.g., an amount of a composition that is effective to treat or prevent pathological conditions, including signs and/or symptoms of disease, associated with a pathogenic organism infection (e.g., spore germination, bacterial growth, toxin production, etc.) in a subject.

As used herein, the term “adjuvant” as used herein refers to an agent which enhances the pharmaceutical effect of another agent.

The expression “amino acid” as used herein is meant to include both natural and synthetic amino acids, and both D- and L-amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present disclosure, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the disclosure.

The term “amino acid” is used interchangeably with “amino acid residue,” and can refer to a free amino acid and to an amino acid residue of a peptide. It will be apparent from the context in which the term is used whether it refers to a free amino acid or a residue of a peptide.

The term “antibody,” as used herein, refers to an immunoglobulin molecule which is able to specifically bind to a specific epitope on an antigen. Antibodies can be derived from natural sources or from recombinant sources and can be intact immunoglobulins or immunoreactive portions of intact immunoglobulins (for example, a fragment or derivative of an antibody that includes an antigen-binding site or a paratope). Antibodies are typically tetramers of immunoglobulin molecules. The antibodies in the present disclosure can exist in a variety of forms including, for example, polyclonal antibodies, monoclonal antibodies, Fv, Fab and F(ab)2, as well as single chain antibodies and humanized antibodies (see e.g., Harlow & Lane (1999) Using Antibodies: A Laboratory Manual, Cold Spring Harbor Laboratory Press, Cold Spring Harbor, New York, United States of America; Harlow & Lane (1988) Antibodies: A Laboratory Manual, Cold Spring Harbor Laboratory Press, Cold Spring Harbor, New York, United States of America; Houston et al. (1988) Proc Natl Acad Sci USA 85:5879-5883; Bird et al. (1988) Science 242:423-426; each of which is incorporated herein by reference in its entirety).

The term “synthetic antibody” as used herein refers to an antibody which is generated using recombinant DNA technology, such as, for example, an antibody expressed by a bacteriophage or a host cell. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antisense oligonucleotide” means a nucleic acid polymer, at least a portion of which is complementary to a nucleic acid which is present in a normal cell or in an affected cell. The antisense oligonucleotides of the disclosure include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (see e.g., U.S. Pat. No. 5,034,506 to Summerton and Weller; Nielsen et al. (1991) Science 254:1497-1500). The term “antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences.

As used herein, the term “biologically active fragments” or “bioactive fragment” of a polypeptide encompasses natural or synthetic portions of the full-length protein that are capable of specific binding to their natural ligand or of performing the function of the protein.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T”, is complementary to the sequence “T-C-A.”

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, a “derivative” of a compound refers to a chemical compound that can be produced from another compound of similar structure in one or more steps, as in replacement of H by an alkyl, acyl, or amino group. Similarly, a “derivative” of a peptide (or of a polypeptide) is a compound that can be produced from or has a biological activity similar to a peptide (or a polypeptide) but that differs in the primary amino acid sequence of the peptide (or the polypeptide) to some degree. By way of example and not limitation, a derivative of a subject peptide of the presently disclosed subject matter is a peptide that has a similar although not identical primary amino acid sequence as the subject peptide (for example, has one or more amino acid substitutions) and/or that has one or more other modifications (e.g., N-terminal, C-terminal, and/or internal modifications) as compared to the subject peptide. Thus, the term “derivative” compasses the term “modified peptide” and vice versa, in the context of peptides. In some embodiments, a derivative of a peptide is a C-terminal amidated peptide.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid of the amino acid sequence, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J Mol Biol 215:403-410) are available for determining sequence identity.

In some embodiments, “identity” can be expressed as a “percent identity”. As used herein, the phrase “percent identity” in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman (1981) 2 Adv Appl Math 482-489; by the homology alignment algorithm disclosed in Needleman & Wunsch (1970) 48 J Mol Biol 443-453; by the search for similarity method disclosed in Pearson & Lipman (1988) Proc Natl Acad Sci USA 85:2444-2448; by computerized implementations of these algorithms (GAP, BESTFIT, FASTA, and TFASTA in the GCGR WISCONSIN PACKAGE®, available from Accelrys, Inc., San Diego, California, United States of America), or by visual inspection. See generally, Altschul et al. (1990) 215 J Mol Biol 403-410; Ausubel et al. (2002) Short Protocols in Molecular Biology, Fifth ed. Wiley, New York, New York, United States of America; and Ausubel et al. (2003) Current Protocols in Molecular Biology, John Wylie & Sons, Inc, New York, New York, United States of America.

One example of an algorithm that is suitable for determining percent sequence identity and sequence similarity is the BLAST algorithm, which is described in Altschul et al. (1990) 215 J Mol Biol 403-410. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al. (1990) 215 J Mol Biol 403-410. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) 89 Proc Natl Acad Sci USA 10915-10919.

In addition to calculating percent sequence identity, the BLAST algorithm also performs a statistical analysis of the similarity between two sequences (see e.g., Karlin & Altschul (1993) 90 Proc Natl Acad Sci USA 5873-5877). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

The term “inhibit”, as used herein, refers to the ability of a compound or any agent to reduce or impede a described function or pathway. For example, inhibition can be by at least 10%, by at least 25%, by at least 50%, by at least 75%, by at least 80%, by at least 85%, by at least 90%, by at least 95%, by at least 97%, by at least 99%, or more.

As used herein, an “instructional material” includes a publication, a recording, a diagram, or any other medium of expression which can be used to communicate the usefulness of the peptide of the disclosure in the kit for effecting alleviation of the various diseases or disorders recited herein. Optionally, or alternately, the instructional material can describe one or more methods of alleviating the diseases or disorders in a cell or a tissue of a mammal. The instructional material of the kit of the disclosure can, for example, be affixed to a container which contains the identified compound disclosure or be shipped together with a container which contains the identified compound. Alternatively, the instructional material can be shipped separately from the container with the intention that the instructional material and the compound be used cooperatively by the recipient.

An “isolated” compound/moiety is a compound/moiety that has been removed from components naturally associated with the compound/moiety. For example, an “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “modulate”, as used herein, refers to changing the level of an activity, function, or process. The term “modulate” encompasses both inhibiting and stimulating an activity, function, or process.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides (e.g., a polypeptide of in some embodiments at least 50 amino acids, in some embodiments at least 75 amino acids, in some embodiments at least 100 amino acids, in some embodiments at least 200 amino acids, in some embodiments at least 300 amino acids, in some embodiments at least 500 amino acids, and in some embodiments more than 500 amino acids).

A peptide encompasses a sequence of 2 or more amino acids wherein the amino acids are naturally occurring or synthetic (non-naturally occurring) amino acids.

The term “linked” or like terms refers to a connection between two entities. The linkage can comprise a covalent, ionic, or hydrogen bond or other interaction that binds two compounds or substances to one another.

As used herein the term “peptidomimetic” refers to a chemical compound having a structure that is different from the general structure of an existing peptide, but that functions in a manner similar to the existing peptide, e.g., by mimicking the biological activity of that peptide. The term “modified peptide” encompasses a peptidomimetic. Peptidomimetics typically comprise naturally-occurring amino acids and/or unnatural amino acids, but can also comprise modifications to the peptide backbone. For example, a peptidomimetic can include one or more of the following modifications:
1. Peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2-S(O)2NR—) linkage, a urea (—NHC(O)NH—) linkage, a —CH2-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C1-C6 alkyl;
2. Peptides wherein the N-terminus is derivatized to a —NRRI group, to a —NRC(O)R group, to a —NRC(O)OR group, to a —NRS(O)2R group, to a —NHC(O)NHR group where R and RI are hydrogen or C1-C6 alkyl with the proviso that R and RI are not both hydrogen;
3. Peptides wherein the C terminus is derivatized to —C(O)R2 where R2 is selected from the group consisting of C1-C6 alkoxy, and —NR3R4 where R3 and R4 are independently selected from the group consisting of hydrogen and C1-C4 alkyl;
4. Modification of a sequence of naturally-occurring amino acids with the insertion or substitution of a non-peptide moiety, e.g., a retroinverso fragment.

The term “permeability”, as used herein, refers to transit of fluid, cell, or debris between or through cells and tissues.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds”, as used herein, is meant a compound which recognizes and binds a specific protein, but does not substantially recognize or bind other molecules in a sample, or it means binding between two or more proteins as in part of a cellular regulatory process, where said proteins do not substantially recognize or bind other proteins in a sample.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein an “amino acid modification” refers in some embodiments to a substitution, addition, or deletion of an amino acid, and includes substitution with, or addition of, any of the 20 amino acids commonly found in human proteins, as well as unusual or non-naturally occurring amino acids such as but not limited to D-amino acids. Commercial sources of unusual amino acids include Sigma-Aldrich (Milwaukee, Wisconsin, United States of America), ChemPep Inc. (Miami, Florida, United States of America), and Genzyme Pharmaceuticals (Cambridge, Massachusetts, United States of America). Unusual amino acids can be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid. The term “modified peptide” encompasses any amino acid modification as described herein.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the disclosure are not limited to products of any of the specific exemplary processes listed herein.

Substitutions can be designed based on, for example, the model of Dayhoff et al. (in Atlas of Protein Sequence and Structure 1978, National Biomedical Research Foundation, Washington D.C., United States of America).

In some embodiments, an amino acid substitution is a conservative amino acid substitution. As used herein, the term “conservative amino acid substitution” is defined in some embodiments as exchanges within one of the following four groups:
I. Small aliphatic, nonpolar or slightly polar residues: Ala, Ser, Thr, Pro, Gly;
II. Polar, charged residues and their amides: Asp, Asn, Glu, Gln, His, Arg, Lys;
III. Large, aliphatic, nonpolar residues: Met, Leu, Ile, Val, Cys;
IV. Large, aromatic residues: Phe, Tyr, Trp.

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al. (1990) Science 247:1306-1310.

For example, the hydropathic index of amino acids may be considered (Kyte & Doolittle (1982) J Mol Biol 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle (1982) J Mol Biol 157:105-132), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet, or reverse turn secondary structure has been determined and is known in the art (see e.g., Chou & Fasman (1974) Biochemistry 13:222-245; Chou & Fasman (1978) Ann Rev Biochem 47:251-276; Chou & Fasman (1979) Biophys J 26:367-384).

Based on such considerations and extensive empirical study, tables of conservative amino acid substitutions have been constructed and are known in the art. By way of example and not limitation, the following substitutions can be made: arginine and lysine; glutamate and aspartate; serine and threonine; glutamine and asparagine; and valine, leucine, and isoleucine.

Alternatively, Table 1 lists exemplary conservative amino acid substitutions.

TABLE 1
Exemplary Conservative Amino Acid Substitutions

| Amino Acid | Possible Substitution(s) |
|---|---|
| Ala (A) | Leu, Ile, Val |
| Arg (R) | Gln, Asn, Lys |
| Asn (N) | His, Asp, Lys, Arg, Gln |
| Asp (D) | Asn, Glu |
| Cys (C) | Ala, Ser |
| Gln (Q) | Glu, Asn |
| Glu (E) | Gln, Asp |
| Gly (G) | Ala |
| His (H) | Asn, Gln, Lys, Arg |
| Ile (I) | Val, Met, Ala, Phe, Leu |
| Leu (L) | Val, Met, Ala, Phe, Ile |
| Lys (K) | Gln, Asn, Arg |
| Met (M) | Phe, Ile, Leu |
| Phe (F) | Leu, Val, Ile, Ala, Tyr |
| Pro (P) | Ala |
| Ser (S) | Thr |
| Thr (T) | Ser |
| Trp (W) | Phe, Tyr |
| Tyr (Y) | Trp, Phe, Thr, Ser |
| Val (V) | Ile, Leu, Met, Phe, Ala |

As used herein, amino acids are represented by the full name thereof, by the three letter code corresponding thereto, or by the one-letter code corresponding thereto, as indicated in the following table (Table 1A):

TABLE 1A
AMINO ACID CODES AND CODONS

| | 3-Letter | 1-Letter | Functionally |
|---|---|---|---|
| Full Name | Code | Code | Equivalent Codons |
| Aspartic Acid | Asp | D | GAC, GAU |
| Glutamic Acid | Glu | E | GAA, GAG |
| Lysine | Lys | K | AAA, AAG |
| Arginine | Arg | R | AGA, AGG, CGA, |
| | | | CGC, CGG, CGU |
| Histidine | His | H | CAC, CAU |
| Tyrosine | Tyr | Y | UAC, UAU |
| Cysteine | Cys | C | UGC, UGU |
| Asparagine | Asn | N | AAC, AAU |
| Glutamine | Gln | Q | CAA, CAG |
| Serine | Ser | S | ACG, AGU, UCA, |
| | | | UCC, UCG, UCU |
| Threonine | Thr | T | ACA, ACC, ACG, ACU |
| Glycine | Gly | G | GGA, GGC, GGG, GGU |
| Alanine | Ala | A | GCA, GCC, GCG, GCU |
| Valine | Val | V | GUA, GUC, GUG, GUU |
| Leucine | Leu | L | UUA, UUG, CUA, CUC |
| Isoleucine | Ile | I | AUA, AUC, AUU |
| Methionine | Met | M | AUG |
| Proline | Pro | P | CCA, CCC, CCG, CCU |
| Phenylalanine | Phe | F | UUC, UUU |
| Tryptophan | Trp | W | UGG |

In some embodiments, another consideration for amino acid substitutions includes whether or not the residue is located in the interior of a protein or is solvent exposed. For interior residues, conservative substitutions can include in some embodiments: Asp and Asn; Ser and Thr; Ser and Ala; Thr and Ala; Ala and Gly; Ile and Val; Val and Leu; Leu and Ile; Leu and Met; Phe and Tyr; Tyr and Trp. For solvent exposed residues, conservative substitutions can include in some embodiments: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, the Dayhoff matrix, the Grantham matrix, the Mclachlan matrix, the Doolittle matrix, the Henikoff matrix, the Miyata matrix, the Fitch matrix, the Jones matrix, the Rao matrix, the Levin matrix, and the Risler matrix (summarized in, for example, Johnson & Overington (1993) J Mol Biol 233:716-738; see also the PROWL resource available at the website of The Rockefeller University, New York, New York, United States of America).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

All the herein disclosed features and embodiments can be implemented individually or in any combination.

Furthermore, the peptide according to the disclosure can also be at least one subunit (module) of a larger peptide or polypeptide, where the polypeptide can comprise a multimer of the sequences described herein, for example 1 to 30 repeats, more preferably 2 to 15 repeats, particularly preferably 2 to 10 repeats, e.g., 2, 3, 4, 5 or 6 repeats of the peptide. The polypeptide may include or consist of such multimers. The term “polypeptide” in this context refers in particular to those peptides that comprise 100 or more amino acids. The term “larger peptides” preferably refers to peptides with at least 40 amino acids, unless otherwise described.

In various embodiments, the peptide is a peptide or polypeptide (multimer) comprising two or more of the peptides as described herein. In various embodiments, the two or more peptides can be connected to one another by at least one spacer, preferably the at least one spacer comprises or consists of 1 to 10 amino acid residues, in particular 2, 3 or 4 amino acid residues, preferably selected from the group consisting of G, P, I, A and S or combinations thereof, in particular GPI or GAS. In such embodiments, the individual peptides are optionally connected linearly to one another via peptide bonds, possibly also via a spacer.

The peptides described herein may have been chemically synthesized in various embodiments and/or recombinantly produced using protein design. Nowadays, short peptides can easily be prepared synthetically, for example using solid-phase synthesis such as Merrifield's solid-phase synthesis. Longer peptides and polypeptides, on the other hand, are often produced recombinantly in the host organism, e.g., in bacteria or yeast.

It is preferred to produce the peptides and/or peptide conjugates according to the disclosure using recombinant processes. This includes all genetic engineering or microbiological processes that are based on the genes for the peptides of interest being introduced into a host organism suitable for production and transcribed and translated by it (summarized in the context of this disclosure as biotechnological processes).

The peptides and/or peptide conjugates according to the disclosure are particularly preferably produced as polypeptides (multimers) and subsequently cleaved into the functional peptides and/or peptide conjugates. Very particularly preferred multimers have 1 to 30 peptide units (each according to the disclosure), each of which is separated from one another by spacers of 1 to 10 amino acids long (e.g., 1, 2, 3 or 4 amino acids). Alternatively, the spacers can also be or include interfaces for specific proteases/peptidases, in particular endopeptidases, or can form such an interface together with parts of the peptide.

Using methods that are generally known today, such as chemical synthesis or the polymerase chain reaction (PCR) in conjunction with standard molecular biological and/or protein chemical methods, it is possible for a person skilled in the art to identify the corresponding nucleic acids and even complete genes using known DNA and/or amino acid sequences to produce. Such methods are, for example, from Sambrook, J., Fritsch, E. F. and Maniatis, T. 2001. Molecular cloning: a laboratory manual, 3rd Edition Cold Spring Laboratory Press. known.

In particularly preferred embodiments, peptides and/or peptide conjugates described herein are produced using biotechnological processes as described above and/or as are known in the art.

The term “expression” refers to the process by which nucleic acid is translated into peptides or is transcribed into RNA, which, for example, can be translated into peptides, polypeptides, or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

The term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

The terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The agents, compounds, compositions, peptides, enzymes, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods for Examples 1-5

Synthetic DNA oligonucleotides were purchased from Integrated DNA Technologies. Restriction endonucleases were purchased from Thermo Fisher Scientific. Q5 high-fidelity DNA polymerase and Taq DNA polymerase were purchased from NEB. Products of PCR and restriction digestion were purified by gel electrophoresis and Syd Laboratories Gel Extraction columns. Plasmid DNA was purified using Syd Laboratories Miniprep columns. DNA sequences were analyzed by Eurofins. Potassium D-luciferin was purchased from Thermo Fisher Scientific. Coelenterazine was purchased from Gold Biotechnology. Furimazine (Nano-Glo®) was purchased from Promega. AkaLumine-HCl was purchased from Aobious. CTZ and DTZ were obtained from GoldBio and Haoyuan Chemexpress, respectively. All other chemicals were purchased from Sigma-Aldrich, Fisher Scientific, or VWR and used without further purification. Bruker Avance DRX 600 and Varian NMRS 600 at the UVA Biomolecular Magnetic Resonance Facility was used to record all NMR spectra. Chemical shift (δ) is given in parts per million relative to 1H (7.24 ppm) and 13C (77.23 ppm) for CDCl₃; 1H (2.50 ppm) and 13C (39.5 ppm) for DMSO-d₆; 1H (3.31 ppm) and 13C (49.15 ppm) for methanol-d₄. Splitting patterns are reported as s (singlet), bs (broad singlet), d (doublet), t (triplet), dd (doublet of doublets), and m (multiplet). Coupling constant (J) is given in Hz. High resolution ESI-MS was run on an Agilent 6545 Q-TOF LC/MS system by direct infusion. A Waters Delta Prep ZQ 2000 LC-MS Purification System equipped with a XBridge BEH Amide OBD Prep Column (130 Å, 5 μm, 30 mm×150 mm) was used for preparative reverse-phase HPLC purifications. Nu/J mice were obtained from the Jackson Laboratory (Cat. #002019) and maintained and treated in standard conditions that complied with all relevant ethical regulations. All animal procedures were approved by the UVA Institutional Animal Care and Use Committee. Images were analyzed using the Fiji image analysis software. Microsoft Excel and GraphPad Prism were used to analyze data and prepare figures.

### Chemical Synthesis of Compounds

### 5-bromo-3-phenylpyrazin-2-amine (1a)

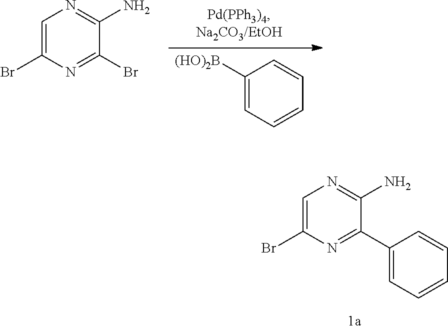 [SMILES: Nc1ncc(Br)nc1Br ; OB(O)c1ccccc1 ; Nc1ncc(Br)nc1-c1ccccc1]

To a solution of Pd (PPh₃) (460 mg, 0.4 mmol, 0.1 equiv.) in 200 mL EtOH was added 2-amino-3,5-dibromopyazine (1010 mg, 4 mmol, 1 equiv.), 1 N Na₂CO₃ solution (8 mL, 8 mmol, 2 equiv.) and phenylboronic acid (490 mg, 4 mmol, 1 equiv.). The resultant mixture was stirred at 80° C. under argon for 12 h. The solvent was removed in vacuo and the residue was suspended in 200 mL ddH₂O, which was extracted twice with EtOAc (200 mL). The organic layers were combined and dried over anhydrous Na₂SO₄, filtered and removed in vacuo. The residue was purified by silica column chromatography with elution (DCM:MeOH=100:1) to yield compound 1a as yellow solid (360 mg, 36%). ¹H NMR (600 MHz, CDCl₃) δ 8.07 (s, 1H), 7.71 (d, J=7.4 Hz, 2H), 7.50 (t, J=7.4 Hz, 2H), 7.45 (t, J=7.4 Hz, 1H), 4.82 (s, 2H). ¹³C NMR (151 MHz, DMSO-d₆) δ 152.4, 142.4, 139.3, 135.9, 129.2, 128.8, 128.7, 128.1, 127.9, 123.9. HRMS (ESI-TOF) calcd for C₁₀H₈BrN₃ [M+H]⁺: 249.9902. found: m/z 249.9916.

### 5-bromo-3-(pyridine-4-yl)pyrazin-2-amine (1b)

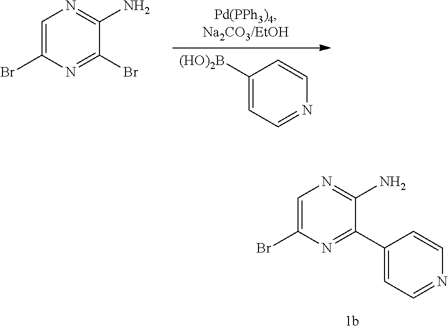 [SMILES: Nc1ncc(Br)nc1Br ; OB(O)c1ccncc1 ; Nc1ncc(Br)nc1-c1ccncc1]

The synthesis of 1b followed the same procedure as for 1a, except that 4-pyridylboronic acid (492 mg, 4 mmol, 1 equiv.) was used. Crude 1b was purified by column chromatography with elution (DCM:MeOH=10:1) to yield 1b as a yellow solid (301 mg, 30%).

¹H NMR (600 MHz, DMSO-d₆) δ 8.69 (d, J=6.0 Hz, 2H), 8.17 (s, 1H), 7.67 (d, J=6.0 Hz, 2H), 6.68 (s, 2H).

¹³C NMR (151 MHz, DMSO-d₆) δ 152.6, 150.1, 144.1, 143.3, 136.1, 124.0, 122.4.

HRMS (ESI-TOF) calcd for C₉H₇BrN₄ [M+H]⁺: 250.9854. found: m/z 250.9845.

### 3-benzyl-5-phenylpyrazin-2-amine (2a)

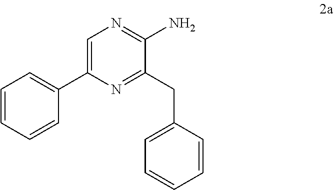 [SMILES: Nc1ncc(-c2ccccc2)nc1Cc1ccccc1]

Compound 2a was prepared following published synthesis methods. ¹H NMR (600 MHz, DMSO-d₆) δ 8.41 (s, 1H), 7.89 (d, J=7.4 Hz, 2H), 7.39 (t, J=7.7 Hz, 2H), 7.33 (d, J=7.6 Hz, 2H), 7.27 (q, J=7.7, 7.1 Hz, 3H), 7.18 (t, J=7.3 Hz, 1H), 6.39 (s, 2H), 4.07 (s, 2H). ¹³C NMR (150 MHz, DMSO-d₆) δ 153.2, 140.5, 139.2, 138.6, 137.6, 137.4, 129.4, 129.1, 128.7, 127.8, 126.6, 125.2, 39.1. HRMS (ESI-TOF) calcd for C₁₇H₁₅N₃ [M+H]⁺: 262.1266. found: m/z 262.1258.

### 3.5-diphenylpyrazin-2-amine (2b)

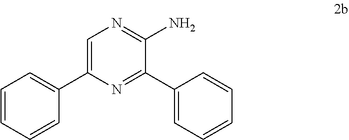 [SMILES: Nc1ncc(-c2ccccc2)nc1-c1ccccc1]

Compound 2b was reported previously.

### 5-phenyl-3-(pyridin-4-yl) pyrazin-2-amine (2c)

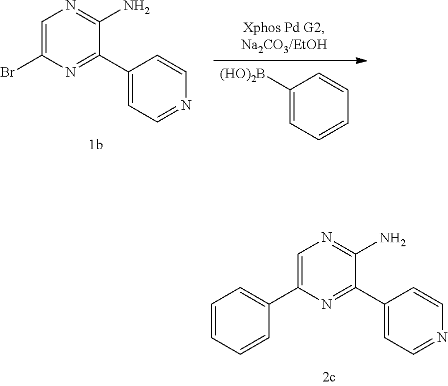 [SMILES: Nc1ncc(Br)nc1-c1ccncc1 ; OB(O)c1ccccc1 ; Nc1ncc(-c2ccccc2)nc1-c1ccncc1]

The synthesis and purification of 2c followed the same procedure as for 2d, except that 1b was used as the starting compound and phenylboronic acid (245 mg, 2 mmol, 2 equiv.) was used as the boron reagent. The product was obtained as a yellow solid (87 mg, 70%). ¹H NMR (600 MHz, DMSO-d₆) δ 7.88-7.84 (m, 2H), 7.70 (s, 1H), 7.15 (dt, J=7.8, 1.2 Hz, 2H), 7.12-7.08 (m, 2H), 6.65-6.59 (m, 2H), 6.57-6.51 (m, 1H). ¹³C NMR (150 MHz, DMSO-d₆) δ 152.3, 150.1, 144.9, 140.0, 139.4, 136.6, 134.7, 128.8, 127.9, 125.0, 122.7. HRMS (ESI-TOF) calcd for C₁₅H₁₂N₄ [M+H]⁺: 249.1062. found: m/z 249.1059.

### 3-phenyl-5-(pyridin-4-yl) pyrazin-2-amine (2d)

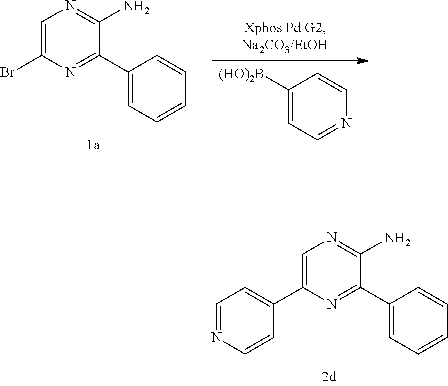 [SMILES: Nc1ncc(Br)nc1-c1ccccc1 ; OB(O)c1ccncc1 ; Nc1ncc(-c2ccncc2)nc1-c1ccccc1]

To a mixture of XPhos Pd G2 (79 mg, 0.1 mmol, 0.2 equiv.) and XPhos (24 mg, 0.05 mmol, 0.1 equiv.) in 5 mL EtOH was added 1a (125 mg, 0.5 mmol, 1 equiv.), 1N Na₂CO₃ (1 mL, 1 mmol, 2 equiv.) and 4-pyridylboronic acid (246 mg, 2 mmol, 4 equiv.). The resulting mixture was stirred at 80° C. under argon for 12 h. The solvent was then removed in vacuo and the residue was dissolved in 1N HCl (30 mL) and subsequently washed with EtOAc (30 mL). The aqueous layer was collected and the pH was then adjusted to 10 by the addition of 1N NaOH. Product 2d precipitated as yellow solid, which was filtered, washed with EtOAc and dried under reduced pressure overnight. (93 mg, 75%). ¹H NMR (600 MHz, DMSO-d₆) δ 8.71 (s, 1H), 8.58 (d, J=6.2 Hz, 2H), 7.94 (d, J=6.2 Hz, 2H), 7.78 (d, J=7.2 Hz, 2H), 7.52 (t, J=7.5 Hz, 2H), 7.46 (t, J=7.3 Hz, 1H), 6.63 (s, 2H). ¹³C NMR (150 MHz, DMSO-d₆) δ 153.3, 150.1, 144.0, 139.2, 138.6, 137.1, 128.8, 128.2, 119.0. HRMS (ESI-TOF) calcd for C₁₅H₁₂N₄ [M+H]⁺: 249.1062. found: m/z 249.1060.

### 4-(5-amino-6-benzylpyrazin-2-yl)phenol (2e)

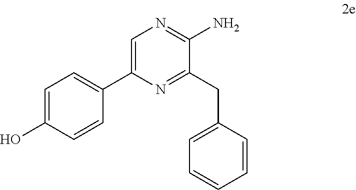 [SMILES: Nc1ncc(-c2ccc(O)cc2)nc1Cc1ccccc1]

Compound 2e was prepared following the published synthesis methods.

¹H NMR (600 MHz, DMSO-d₆) δ 9.49 (s, 1H), 8.29 (s, 1H), 7.72 (d, J=8.6 Hz, 2H), 7.33 (d, J=7.3 Hz, 2H), 7.28 (t, J=7.6 Hz, 2H), 7.18 (t, J=7.3 Hz, 1H), 6.79 (d, J=8.6 Hz, 2H), 6.19 (s, 2H), 4.06 (s, 2H). ¹³C NMR (150 MHz, DMSO-d₆) δ 157.1, 152.0, 139.7, 139.5, 138.3, 135.9, 135.9, 128.9, 128.2, 126.2, 126.1, 115.5, 115.4, 38.7. HRMS (ESI-TOF) calcd for C₁₇H₁₅N₃₀ [M+H]⁺: 278.1215. found: m/z 278.1208.

### 3-phenyl-5-(pyridin-3-yl) pyrazin-2-amine (2f)

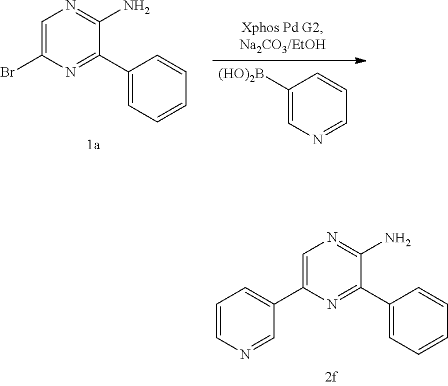 [SMILES: Nc1ncc(Br)nc1-c1ccccc1 ; OB(O)c1cccnc1 ; Nc1ncc(-c2cccnc2)nc1-c1ccccc1]

The synthesis and purification of 2f (above) followed the same procedure as 2d, whereas 3-pyridylboronic acid (246 mg, 2 mmol, 4 equiv.) was used. Yellow solid (95 mg, 77%). ¹H NMR (600 MHz, DMSO-d₆) δ 9.19 (s, 1H), 8.64 (s, 1H), 8.54 (d, J=3.9 Hz, 1H), 8.39 (d, J=8.1 Hz, 1H), 7.79 (d, J=7.3 Hz, 2H), 7.53-7.49 (m, 3H), 7.46 (t, J=7.3 Hz, 1H), 6.47 (s, 2H). ¹³C NMR (151 MHz, DMSO-d₆) δ 152.6, 148.5, 146.3, 138.4, 138.4, 137.3, 137.2, 132.6, 132.3, 132.2, 128.8, 128.6, 128.3. HRMS (ESI-TOF) calcd for C₁₅H₁₂N₄ [M+H]⁺: 249.1062. found: m/z 249.1060.

### 3-pyridin-4-yl-1,1-diethoxyacetone (+)

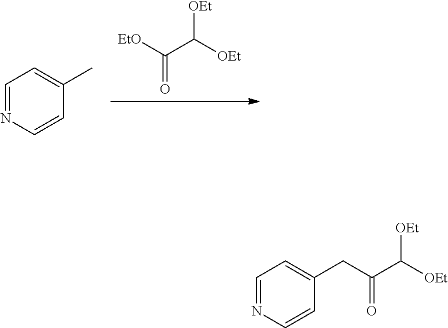

To a solution of 4-methylpyridine (931 mg, 10 mmol, 1 equiv.) in 50 mL anhydrous THF was added potassium tert-butoxide (5.6 g, 50 mmol, 5 equiv.), and the mixture was stirred at room temperature for 10 min. Ethyl diethoxyacetate (3.52 g, 20 mmol, 2 equiv.) in 20 mL THF was then added dropwise over 10 min. The resulting mixture was stirred overnight, and solvent was removed under vacuo. The residue was purified by silica column chromatography with elution (Hexane:EtOAc=1:3 to 100% EtOAc) to yield product as light yellow solid (669 mg, 30%). 1H NMR (600 MHz, DMSO-d₆) δ 8.47 (d, J=5.8 Hz, 2H), 7.18 (d, J=5.8 Hz, 2H), 4.81 (s, 1H), 3.91 (s, 2H), 3.63 (dq, J=9.7, 7.1 Hz, 2H), 3.54 (dq, J=9.7, 7.1 Hz, 2H), 1.15 (t, J=7.1 Hz, 6H). ¹³C NMR (150 MHz, DMSO-d₆) δ 202.0, 149.3, 143.2, 125.3, 101.6, 62.8, 42.6, 15.1. HRMS (ESI-TOF) calcd for C₁₂H₁₇NO₃ [M+H]⁺: 224.1208. found: m/z 224.1195.

### 8-benzyl-6-phenyl-2-(pyridin-4-ylmethyl)imidazo[1,2-a]pyrazin-3(7H)-one (3a)

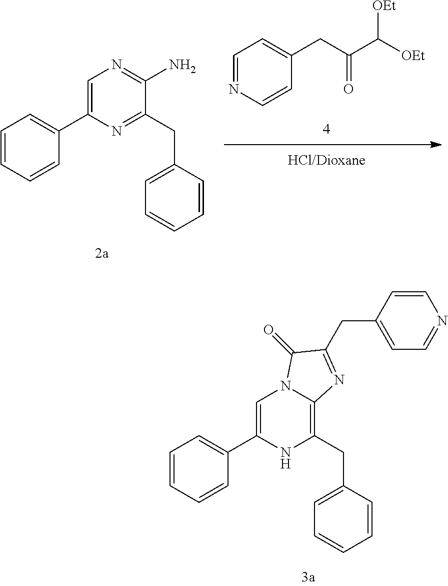 [SMILES: Nc1ncc(-c2ccccc2)nc1Cc1ccccc1 ; CCOC(OCC)C(=O)Cc1ccncc1 ; O=c1c(Cc2ccncc2)nc2c(Cc3ccccc3)[nH]c(-c3ccccc3)cn1-2]

To a solution of 2a (26 mg, 0.1 mmol, 1 equiv.) and 4 (89 mg, 0.4 mmol, 4 equiv.) in 5 mL degassed 1,4-dioxane was added 0.8 mL 6N HCl. The resulting mixture was stirred at 80° C. in a sealed tube for 12 h. The solvent was then removed in vacuo and the residue was dissolved in 1 mL (ACN:H₂O=1:1) and next purified with preparative RP-HPLC (acetonitrile/water=1:99 to 90:10, 20 mL/min, UV 254 nm). Product fractions were combined and lyophilized to give 3a as yellow powder (15 mg, 38%), which has to be stored as solid at −80° C. for long-term stability. ¹H NMR (600 MHz, Methanol-d₄) δ 8.43 (d, J=6.2 Hz, 2H), 7.74 (s, 1H), 7.65 (d, J=6.8 Hz, 2H), 7.49-7.38 (m, 7H), 7.29 (t, J=7.6 Hz, 2H), 7.23 (t, J=7.4 Hz, 1H), 4.42 (s, 2H), 4.23 (s, 2H). ¹³C NMR (150 MHz, Methanol-d₄) δ 142.6, 137.9, 131.1, 130.4, 130.0, 129.9, 129.1, 128.5, 128.3, 110.1, 49.7. HRMS (ESI-TOF) calcd for C₂₅H₂₀N₄O [M+H]⁺: 393.1637. found: m/z 393.1630.

### 6,8-diphenyl-2-(pyridin-4-ylmethyl)imidazo[1.2-a]pyrazin-3 (7H)-one (3b)

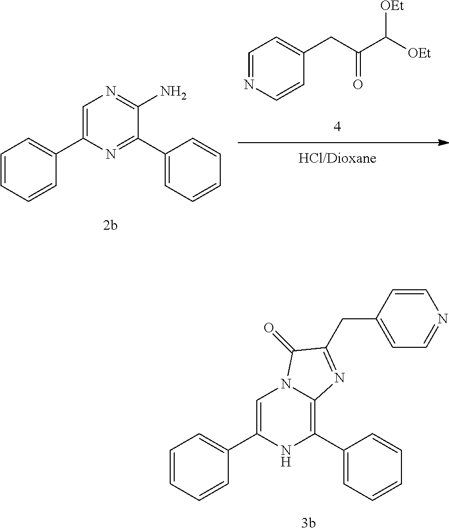 [SMILES: Nc1ncc(-c2ccccc2)nc1-c1ccccc1 ; CCOC(OCC)C(=O)Cc1ccncc1 ; O=c1c(Cc2ccncc2)nc2c(-c3ccccc3)[nH]c(-c3ccccc3)cn1-2]

The synthesis and purification of 3b (above) followed the same procedure as 3a, whereas 2b (25 mg, 0.1 mmol, 1 equiv.) was used as the starting compound. Orange powder (8 mg, 21%). ¹H NMR (600 MHz, Methanol-d₄) δ 8.79 (d, J=6.5 Hz, 2H), 8.50 (s, 1H), 8.15 (d, J=7.2 Hz, 2H), 8.06 (d, J=6.5 Hz, 2H), 8.00 (d, J=7.4 Hz, 2H), 7.69 (t, J=7.4 Hz, 1H), 7.65 (t, J=7.2 Hz, 2H), 7.58 (t, J=7.2 Hz, 2H), 7.56-7.53 (m, 1H), 4.66 (s, 2H). ¹³C NMR (150 MHz, Methanol-d₄) δ 161.0, 146.2, 142.7, 139.3, 134.7, 133.3, 133.0, 131.3, 131.0, 130.4, 130.3, 128.8, 128.5, 112.2. HRMS (ESI-TOF) calcd for C₂₄H₁₈N₄O [M+H]⁺: 379.1481. found: m/z 379.1480.

### 2-benzyl-8-phenyl-6-(pyridin-4-yl)imidazo[1,2-a]pyrazin-3 (7H)-one (3c)

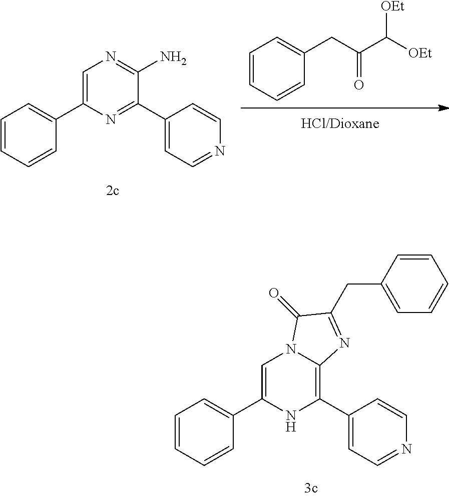 [SMILES: Nc1ncc(-c2ccccc2)nc1-c1ccncc1 ; CCOC(OCC)C(=O)Cc1ccccc1 ; O=c1c(Cc2ccccc2)nc2c(-c3ccncc3)[nH]c(-c3ccccc3)cn1-2]

To a solution of 2c (25 mg, 0.1 mmol, 1 equiv.) and 1,1-diethoxy-3-phenylpropan-2-one (89 mg, 0.4 mmol, 4 equiv.) in 5 mL degassed 1,4-dioxane was added 0.8 mL 6 N HCl, and the resulting mixture was stirred at 80° C. in a sealed tube for 12 h. The solvent was then removed in vacuo and the residue was dissolved in 1 mL (ACN:H₂O=1:1) and next purified with preparative RP-HPLC (acetonitrile/water=1:99 to 90:10, 20 mL/min, UV 254 nm). Product fractions were combined and lyophilized to give 3c as brown powder (9 mg, 23%). ¹H NMR (600 MHz, Acetonitrile-d₃ and D₂O, ratio=9:1) δ 9.31 (d, J=6.8 Hz, 2H), 8.84 (d, J=6.8 Hz, 2H), 8.54 (s, 1H), 8.09 (d, J=8.0 Hz, 2H), 7.52 (t, J=7.6 Hz, 2H), 7.44 (t, J=7.3 Hz, 1H), 7.35 (d, J=7.7 Hz, 2H), 7.28 (t, J=7.7 Hz, 2H), 7.24-7.21 (m, 1H), 4.19 (s, 2H). ¹³C NMR (150 MHz, Methanol-d₄) δ 143.0, 140.7, 140.1, 139.5, 137.5, 132.0, 130.2, 129.9, 129.6, 129.4, 127.8, 127.5, 127.4, 126.5, 113.8, 33.6. HRMS (ESI-TOF) calcd for C₂₄H₁₈N₄O [M+H]⁺: 379.1481. found: m/z 379.1477.

### 2-benzyl-6-phenyl-8-(pyridin-4-yl)imidazo[1,2-a]pyrazin-3(7H)-one (3d)

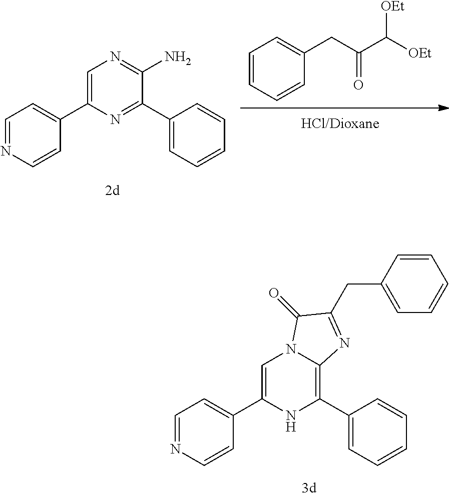

The synthesis and purification of 3d followed the same procedure as 3c, except that 2d (25 mg, 0.1 mmol, 1 equiv.) was used as the starting compound. The product was obtained as yellow powder (6 mg, 16%). ¹H NMR (600 MHz, Methanol-d₄) δ 9.52 (s, 1H), 9.01 (d, J=6.8 Hz, 2H), 8.97 (d, J=6.8 Hz, 2H), 8.15-8.11 (m, 2H), 7.71-7.66 (m, 3H), 7.35-7.30 (m, 4H), 7.25 (t, J=7.4 Hz, 1H), 4.36 (s, 2H). ¹³C NMR (150 MHz, Methanol-d₄) δ 154.5, 148.8, 143.5, 140.4, 138.4, 137.2, 135.0, 133.1, 130.6, 130.4, 130.4, 130.1, 129.8, 129.5, 128.7, 128.3, 125.3, 117.2, 30.5. HRMS (ESI-TOF) calcd for C₂₄H₁₈N₄O [M+H]⁺: 379.1481. found: m/z 379.1476.

### 8-benzyl-6-(4-hydroxyphenyl)-2-(pyridin-4-ylmethyl)imidazo[1,2-a]pyrazin-3 (7H)-one (3e)

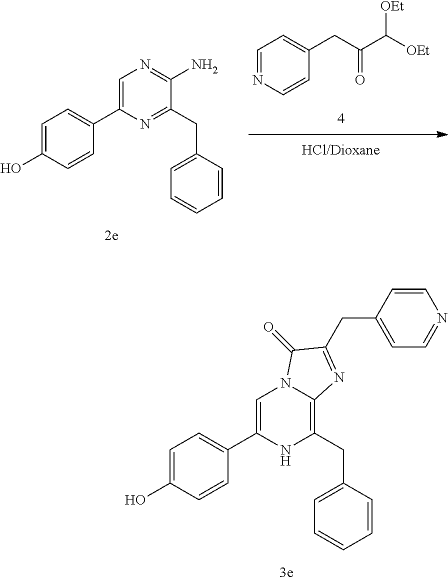 [SMILES: Nc1ncc(-c2ccc(O)cc2)nc1Cc1ccccc1 ; CCOC(OCC)C(=O)Cc1ccncc1 ; O=c1c(Cc2ccncc2)nc2c(Cc3ccccc3)[nH]c(-c3ccc(O)cc3)cn1-2]

The synthesis and purification of 3e followed the same procedure as 3a, except that 2e (28 mg, 0.1 mmol, 1 equiv.) was used as the starting compound. The product was obtained as yellow powder (14 mg, 34%). ¹H NMR (600 MHz, Methanol-d₄) δ 8.78 (d, J=5.2 Hz, 2H), 8.08 (d, J=5.2 Hz, 2H), 8.01 (s, 1H), 7.72 (d, J=7.7 Hz, 2H), 7.53 (d, J=7.7 Hz, 3H), 7.42 (d, J=7.4 Hz, 2H), 7.31 (t, J=7.4 Hz, 2H), 7.25 (t, J=7.7 Hz, 1H), 4.58 (s, 2H), 4.52 (s, 2H). ¹³C NMR (150 MHz, Methanol-d₄) δ 161.2, 142.6, 137.1, 130.3, 130.2, 130.1, 129.1, 128.7, 117.3, 111.3. HRMS (ESI-TOF) calcd for C₂₅H₂₀N₄O₂ [M+H]⁺: 409.1586. found: m/z 409.1585.

### 2-benzyl-8-phenyl-6-(pyridin-3-yl)imidazo[1,2-a]pyrazin-3(7H)-one (3f)

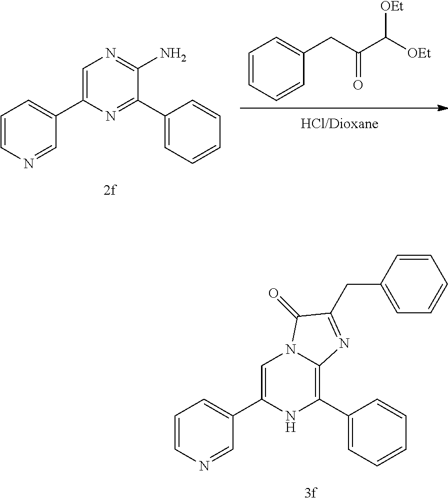

The synthesis and purification of 3f followed the same procedure as 3c, except that 2f (25 mg, 0.1 mmol, 1 equiv.) was used as the starting compound. The product was obtained as orange powder (8 mg, 21%). ¹H NMR (600 MHz, Methanol-d₄) δ 9.73 (s, 1H), 9.47 (d, J=8.3 Hz, 1H), 9.36 (s, 1H), 8.99 (d, J=5.6 Hz, 1H), 8.32-8.27 (m, 1H), 8.09 (d, J=6.6 Hz, 2H), 7.72-7.66 (m, 3H), 7.32 (7.67-7.30, 4H), 7.25 (t, J=7.0 Hz, 1H), 4.36 (s, 2H). ¹³C NMR (150 MHz, Methanol-d₄) δ 148.6, 145.3, 142.9, 141.5, 138.3, 137.4, 137.2, 134.8, 133.1, 130.5, 130.4, 130.1, 129.5, 129.1, 128.3, 128.2, 121.6, 114.8, 30.3. HRMS (ESI-TOF) calcd for C₂₄H₁₈N₄O [M+H]⁺: 379.1481. found: m/z 379.1478.

### Plasmid and Library Construction

Polymerase chain reactions (PCRs) with various synthetic oligonucleotide pairs were used to amplify genetic elements. To create a gene library with randomization at residues 20 and 21, oligo pairs pBAD-F and L20D21NNK-R, L20D21NNK-F and pBAD-R, were used to amplify two individual fragments from pBAD-teLuc; the corresponding products were used for assembly in a subsequent overlap PCR reaction by using oligos pBAD-F and pBAD-R. The assembled full-length fragment was digested with Xho I and Hind III restriction enzymes and ligated into a predigested, compatible pBAD/His B plasmid. Similarly, pBAD-F, 29VSSNNK-R, 29VSSNNK-F, and pBAD-R were used to create a library with randomization at residues 29, 30, and 31. To introduce random mutations across the gene, Taq DNA polymerase was used in all reactions with 0.2 mM MnCl2 along with unbalanced dNTPs to promote amplification errors. To create mammalian expression plasmids, HindIII-pyr-F-Koz and pyr-R-XhoI were used to amplify the LumiLuc gene fragment, which was further treated with Hind III and Xho I restriction enzymes and ligated into a predigested, compatible pcDNA3 plasmid. The Akaluc gene was synthesized by Eurofins, and cloned into a pBAD plasmid for bacterial expression and a pcDNA3 plasmid for mammalian expression, by using Aka-F-XhoI and Aka-R-HindIII or Aka-F-HindIII-Kozak and Aka-R-XhoI oligonucleotides. To build mScarlet-LumiLuc fusion library, mScarlet-F-XhoI and mScar-NNK-pyr-R oligonucleotides were used to amplify mScarlet-I gene, while mScar-NNK-pyr-F and pyr-R-HindIII oligonucleotides were used for LumiLuc cloning, which were subsequently assembled by overlap PCR reaction. The product was digested with Xho I and Hind III restriction enzymes and ligated into a predigested, compatible pBAD/His B plasmid. The LumiScarlet gene was cloned into pcDNA3 for mammalian expression using HindIII-mScarlet-F-Koz and pyr-R-XhoI oligonucleotides. All ligation products were used to transform Escherichia coli DH10B electrocompetent cells, which were next plated on LB agar plates supplemented with ampicillin (100 μg/mL).

### Library Screening

DH10B cells containing luciferase mutants were plated on LB agar plates supplemented with ampicillin (100 μg/mL) and L-arabinose (0.02%, w/v) and incubated at 37° C. overnight to form bacterial colonies. Agar plates were left at room temperature for another 6 hours, and this was followed by bioluminescence imaging using a luminescence dark box (UVP Bio Spectrum) equipped with a QSI 628 cooled CCD camera (Quantum Scientific Imaging). Digital images were acquired after spraying about 200 μL of 10 UM substrates to each agar plate, and next, images were processed with the Fiji image analysis software to derive bioluminescence intensities of individual colonies.

For each round of selection, the brightest 20 colonies from a total of about 10,000 colonies were chosen and inoculated in 5 mL liquid LB broth containing ampicillin (100 μg/mL) and L-arabinose (0.02%, w/v). After overnight growth at 37° C. and 250 rpm, the cultures were moved onto a shaker at room temperature for another 6 hours. 500 μL cell cultures were centrifuged and next lysed with 100 μL B-PER (Thermo Fisher Scientific). Next, to 1 μL lysate from each sample was added 100 μL substrate at a final concentration of 20 μM in assay buffer. Bioluminescence activities of individual samples were measured on a Synergy Mx Microplate Reader (BioTek). Kinetics were followed for 0.1 s signal integration every 60 s for a total of 20 min.

Top three mutants showing exceptionally high bioluminescence activities or extended kinetics were chosen for next-round selection, sequencing, and other additional characterization. mScarlet-I and LumiLuc fusion libraries were screened for high BRET efficiency using a 600-700 nm bandpass filter. 20 colonies were picked from each library and inoculated in 5 mL liquid LB broth containing ampicillin (100 μg/mL) and L-arabinose (0.02%, w/v). The cell lysates were prepared with B-PER and the bioluminescence emission spectra were measured by adding 20 μM 8pyDTZ. The construct showing highest BRET efficiency was designated LumiScarlet.

### In Vitro Bioluminescence Characterization

Luciferases were expressed and purified as previously described. A Synergy Mx Microplate Reader (BioTek) was used for all in vitro bioluminescence characterizations. 50 μL of luciferin substrates was injected into the wells of white 96-well plates containing 50 μL of pure enzymes in assay buffer (1 mM CDTA, 0.5% Tergitol NP-40, 0.05% Antifoam 204, 150 mM KCl, 100 mM MES pH 6.0, 1 mM DTT, and 35 mM thiourea). The final concentrations of all enzymes were 20 pM. Measurements were taken every 30 s post injection (0.1 s integration and 10 s shaking during intervals). Akaluc bioluminescence assays were performed at final concentration of 10 nM Akaluc and 100 μM AkaLumine in an assay buffer containing 30 mM MOPS (pH 7.0), 1.5 mM ATP, and 5 mM MgSO₄. To derive values for apparent Michaelis constants (Km), substrate concentrations varied from 0.78 to 50 μM, and peak bioluminescence intensities at individual substrate concentrations were used to fit the Michaelis-Menten equation. To record emission spectra, 50 μL of 20 μM substrates were injected into 50 μL of 2 nM pure enzymes, and the bioluminescence spectra were collected with 0.1 s integration and 1 nm increments from 350 to 750 nm.

### Chemiluminescence Measurement

0.63 g ammonium bicarbonate was dissolved in 12 mL water and 24 mL acetonitrile containing 30% aqueous hydrogen peroxide, resulting in an active peroxymonocarbonate solution. The solution was left at room temperature for 10 min. Each stock solution containing synthetic analogues (500 μM, 100 μL) was dispensed into wells of a 96-well plate, and chemiluminescence was triggered by addition of 100 μL of the peroxymonocarbonate solution. Light emission was recorded on a Synergy Mx Microplate Reader (BioTek) with 0.1 s integration and 1 nm increments from 350 to 750 nm.

### Mammalian Cell Culture, Transfection, and Imaging

HEK 293T cells were cultured and transfected as previously described. The number and density of cells in Dulbecco's phosphate-buffered saline (DPBS) were determined using a hemocytometer. Cells were next diluted in DPBS to gain the desired numbers in each 50 μL solution. To use the luminescence dark box to directly image cells, we added luciferase-expressing HEK 293T cells (5,000 cells per well with ˜70% transfection efficiency) and the corresponding luciferin substrates into wells of a white-wall, 96-well plate. Bioluminescence was imaged using a luminescence dark box immediately after substrate addition. The camera exposure time was set at 2 s. A Chroma Red 600-700 nm filter was used to acquire far-red emission. All images were analyzed using the Fiji image analysis software.

### Generation of Luciferase-Expressing Stable Cell Lines

Hela cells were cultured at 37° C. with 5% CO₂ in Dulbecco's Modified Eagle's Medium (DMEM) supplemented with 10% fetal bovine serum (FBS). Hela cells were transfected with pcDNA3-teLuc, pcDNA3-Antares2, pcDNA3-LumiLuc, pcDNA3-LumiScarlet, or pcDNA3-Akaluc as previously described. Forty-eight hours after transfection, cells were passed into fresh DMEM containing 10% FBS and 1 mg/mL G418. The medium was removed and replaced every 3 days. Stable polyclonal cell lines were generated after approximately 2 weeks of G418 selection.

### Xenograft Mouse Model

Hela cells stably expressing luciferases were dissociated with trypsin and re-suspended in 10 mL DMEM. Cell numbers were determined using a hemocytometer, and cell viability was determined using a trypan blue exclusion test. 10⁴ or 10⁵ cells were re-suspended in 100 μL FBS-free DMEM containing 50% Matrigel matrix (Corning). 8-week-old female nude mice were first anesthetized using isoflurane. Cells were subcutaneously injected into the left and right dorsolateral trapezius regions or thoracolumbar regions. Mice were recovered on heat pads for 5 min while cells were allowed to settle. On day 1, 3, 5, 7, 14, and 28 post tumor implants, mice were subsequently imaged using a Caliper IVIS Spectrum (Perkin Elmer) approximately 5 min after intravenous (i.v.) administration of corresponding luciferins (100 μL solution for indicated doses). DTZ was dissolved in a 100 μL solution containing 8% glycerol, 10% ethanol, 10% hydroxypropyl-β-cyclodextrin, and 35% PEG 400 in water. 8pyDTZ and AkaLumine-HCl were dissolved in normal saline. All solutions were passed through 0.22 μm pore filters before administrations. The following conditions were used for image acquisition: open filter for total bioluminescence, exposure time=60 s (Day 1, 3, and 5); 30 s (Day 7); 10 s (Day 14); 3 s (Day 28), binning=small, field of view=21.6×21.6 cm, and f/stop=1. Image analysis was performed using the Living Image 4.3.1 software.

### Deep-Tissue Mouse Model

10⁶ Hela cells stably expressing either LumiLuc, LumiScarlet or Akaluc were i.v. injected to female nude mice. After 4 h, images were acquired using a Caliper IVIS Spectrum immediately after i.v. delivery of 0.2 μmol 8pyDTZ or 1.5 μmol AkaLumine-HCl in 100 μL normal saline. The following conditions were used for image acquisition: open filter for total bioluminescence, exposure time=10 s, binning=small, field of view=21.6×21.6 cm, and f/stop=1.

### Fluorescence Imaging of ATP in Mammalian Cells

HEK293T and HeLa cells were cultured and transfected as aforementioned. Images were acquired on a Leica DMi8 inverted microscope equipped with the SPE confocal module. Cells were cultured in DMEM (no phenol red) with 4.5 g/L glucose. 405 and 488 nm laser was used to excite PercevalHR, and emission was collected from 510 nm to 600 nm. Intervals between each image were 5 seconds. 50 μM 8pyDTZ was added to cells. For the internal calibration purpose, iodoacetic acid (IAA) was added to a final concentration of 5 mM to completely deplete intracellular ATP. pHRFP was also used to monitor the cellular pH change before and after addition of 50 μM 8pyDTZ.

### Statistical Analysis

Unpaired two-tailed t-tests were used to determine all p-values. No statistical methods were used to predetermine the sample size. Animals were randomly assigned to receive various treatments. Unless otherwise indicated, data are shown as mean±s.d., and error bars in figures represent s.d.

### Example 1: Design and Synthesis of Pyridyl CTZ and DTZ Analogs with Enhanced Water Solubility

Despite that recent studies have synthesized and tested a number of CTZ analogs with NanoLuc, the luciferase has not yet been optimized to pair with these new substrates and the water solubility issue of the substrates has not yet been tackled systematically. Thus, a need remained for CTZ and DTZ analogs with improved water solubility.

The chemical structures of coelenterazine (CTZ), furimazine (FRZ) and diphenylterazine (DTZ) are provided below.

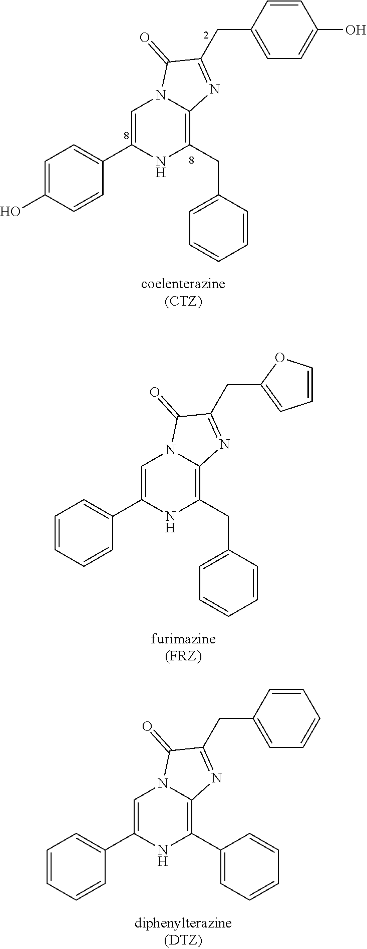 [SMILES: O=c1c(Cc2ccc(O)cc2)nc2c(Cc3ccccc3)[nH]c(-c3ccc(O)cc3)cn1-2 ; O=c1c(Cc2ccco2)nc2c(Cc3ccccc3)[nH]c(-c3ccccc3)cn1-2 ; O=c1c(Cc2ccccc2)nc2c(-c3ccccc3)[nH]c(-c3ccccc3)cn1-2]

The need was fulfilled, as disclosed herein, by using the concept of bioisostere replacements in medicinal chemistry. Pyridine is considered a biocompatible N-heterocycle substituent for benzene with enhanced water solubility, because pyridine-containing molecules can be readily turned into pyridinium salts. Therefore, a convergent synthetic route was designed to prepare a series of CTZ and DTZ analogs with pyridyl isomer substitutions at the C-2, C-6 and C-8 positions of the imidazopyrazinone core (Scheme 1, below). Briefly, Suzuki or Negishi cross-coupling reactions were first used to regioselectively functionalize 2-amino-3,5-dibromopyrazine with either pyridyl, phenyl, or benzyl functional groups to give monosubstituted products (1a-c), which were subsequently derivatized via Suzuki cross-coupling reactions to afford disubstituted intermediates (2a-f, see structures and synthetic methods above). In the second cross-coupling step, the XPhos-Pd-G2 catalyst was used to enhance reaction yields and minimize the protodeboronation of pyridyl boronic acids. An acid-catalyzed ring closing reaction in dioxane was also utilized to derive various pyridyl CTZ and DTZ analogs (3a-f, Table 2) from the disubstituted intermediates and corresponding α-ketoacetals.

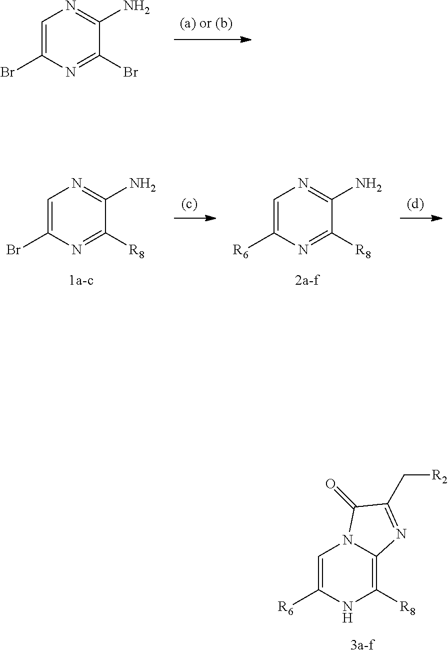

Scheme I. Synthesis of pyridyl CTZ and DTZ analogs. (a) Suzuki coupling: Pd(PPh₃)₄, Na₂CO₃, R₈-B(OH)₂, and EtOH; (b) Negishi coupling: PhCH₂MgCl, ZnCl₂, (PPh₃)₂PdCl₂, and THF; (c) Suzuki coupling: XPhos-Pd-G2, Na₂CO₃, R₆-B(OH)₂, and EtOH; (d) Acid-catalyzed ring closing: corresponding α-ketoacetal, HCl, and dioxane.

Turbidimetric solubility assays were used to evaluate water solubility of these CTZ and DTZ analogs (Table 2). Surprisingly, the newly synthesized pyridyl analogs enhanced the solubility by 4- to 14-fold as compared to CTZ and DTZ. The autoluminescence and stability of these new analogs was also evaluated, the results of which indicated that they are comparable or even better than CTZ and FRZ. Moreover, their chemiluminescence was also evaluated, because the wavelength of bioluminescence is often related to the wavelength of substrate chemiluminescence, although the luciferase enzyme provides further electrostatic tuning which can reshape the emission. In addition, the bioluminescence of these new substrates in the presence of several representative, ATP-independent luciferases such as RLuc8, NanoLuc, teLuc, and aequorin was evaluated.

Although each luciferase has different substrate preferences, the compound 3a (pyCTZ) generated strong blue bioluminescence in the presence of each of these tested luciferases. When paired with aequorin, the bioluminescence intensity of pyCTZ was comparable to native CTZ, suggesting that pyCTZ may be directly used to replace CTZ for aequorin-based calcium sensing. Furthermore, compared to DTZ, compounds 3c (8pyDTZ), and 3f were able to emit red-shifted chemiluminescence and/or bioluminescence, while 3b and 3d (6pyDTZ) caused hypsochromic shift (Table 2). Molecular mechanisms governing the spectral shift properties of these synthetic substrates remain to be investigated. Because 3c showed the most red-shifted emission and red-shifted photons can penetrate through tissue better, 3c (8pyDTZ) was selected as the candidate substrate for further development of an optimized, red-shifted luciferase-luciferin pair.

TABLE 2
CHEMICAL AND PHOTOLUMINESCENCE PROPERTIES OF SYNTHETIC PYRIDYL CTZ AND
DTZ ANALOGS.

| | | | | Bio- | Chemi- | Water |
|---|---|---|---|---|---|---|
| | | | | luminescence | luminescence | Solubility |
| Compound | R₆ | R₈ | R₂ | ^{a}λₘₐₓ (nm) | ^{b}λₘₐₓ (nm) | (μM) |
| 3a | 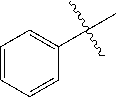 [SMILES: *c1ccccc1] | 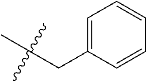 [SMILES: *Cc1ccccc1] | 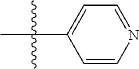 [SMILES: *c1ccncc1] | 451 | 505 | 1416 |
| 3b | 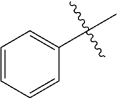 [SMILES: *c1ccccc1] | 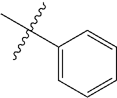 [SMILES: *c1ccccc1] | 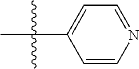 [SMILES: *c1ccncc1] | 497 | 506 | 1813 |
| 3c | 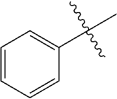 [SMILES: *c1ccccc1] | 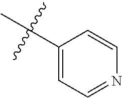 [SMILES: *c1ccncc1] | 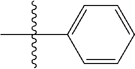 [SMILES: *c1ccccc1] | 532 | 555 | 1711 |
| 3d | 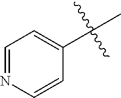 [SMILES: *c1ccncc1] | 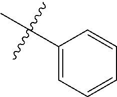 [SMILES: *c1ccccc1] | 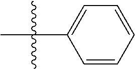 [SMILES: *c1ccccc1] | 483 | 492 | 1736 |
| 3e | 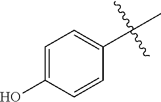 [SMILES: *c1ccc(O)cc1] | 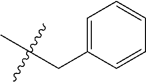 [SMILES: *Cc1ccccc1] | 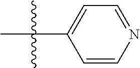 [SMILES: *c1ccncc1] | 450 | 465 | 987 |
| 3f | 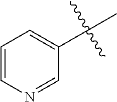 [SMILES: *c1cccnc1] | 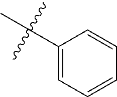 [SMILES: *c1ccccc1] | 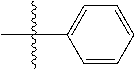 [SMILES: *c1ccccc1] | 518 | 503 | 1562 |
| CTZ | 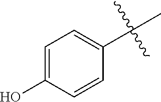 [SMILES: *c1ccc(O)cc1] | 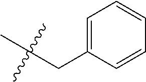 [SMILES: *Cc1ccccc1] | 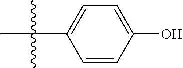 [SMILES: *c1ccc(O)cc1] | 455 | 461 | 256 |
| DTZ | 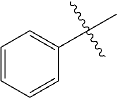 [SMILES: *c1ccccc1] | 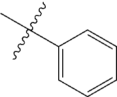 [SMILES: *c1ccccc1] | 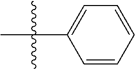 [SMILES: *c1ccccc1] | 502 | 510 | 131 |

Note:
^{a}Determined with 1 nM teLuc in PBS;
^{b}Triggered by peroxymonocarbonate formed in situ.

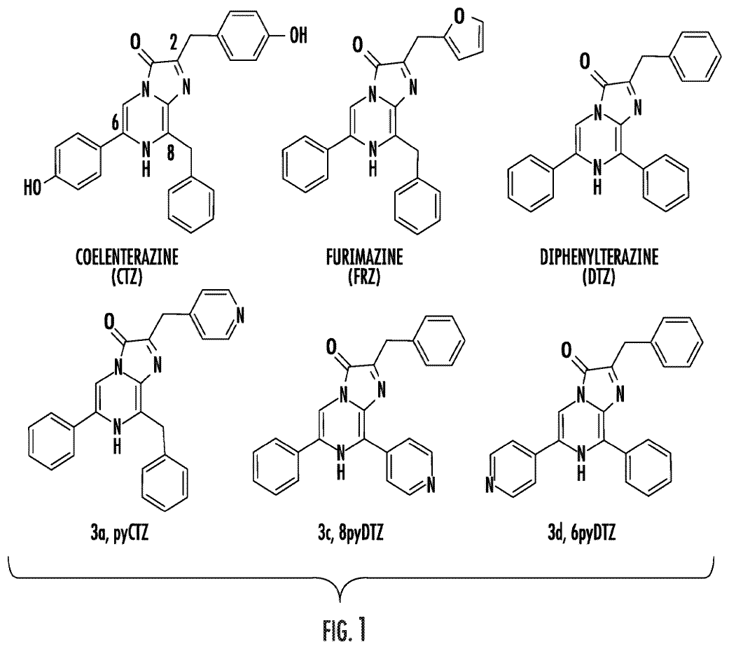

The chemical structures of pyCTZ (3a in Table 2), 8pyDTZ (3c in Table 2) and 6pyDTZ (3d in Table 2) are provided below, as well as in FIG. 1.

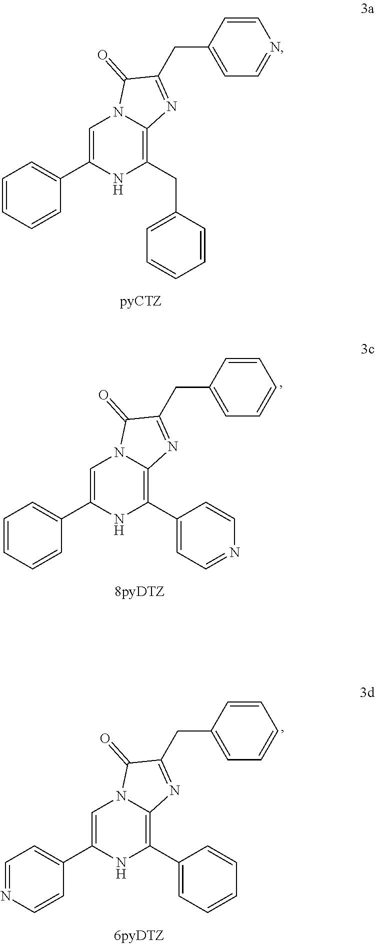 [SMILES: O=c1c(Cc2ccncc2)nc2c(Cc3ccccc3)[nH]c(-c3ccccc3)cn1-2 ; O=c1c(Cc2ccccc2)nc2c(-c3ccncc3)[nH]c(-c3ccccc3)cn1-2 ; O=c1c(Cc2ccccc2)nc2c(-c3ccccc3)[nH]c(-c3ccncc3)cn1-2]

### Example 2: Directed Evolution of the teLuc Luciferase for Improved Brightness

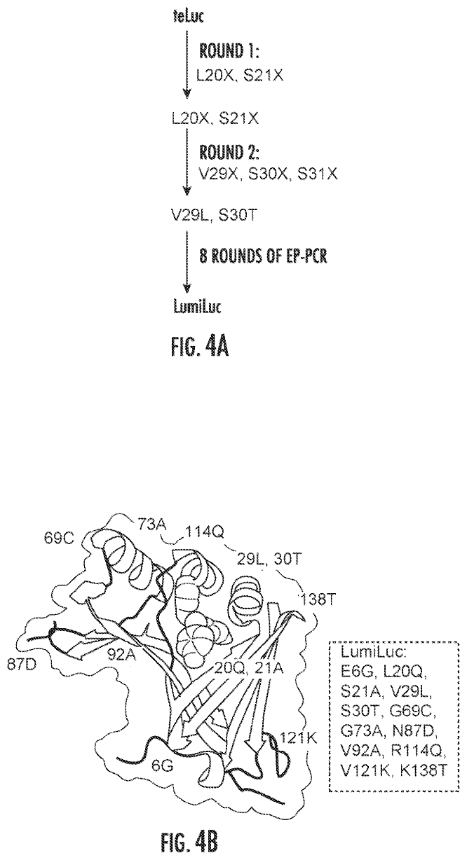

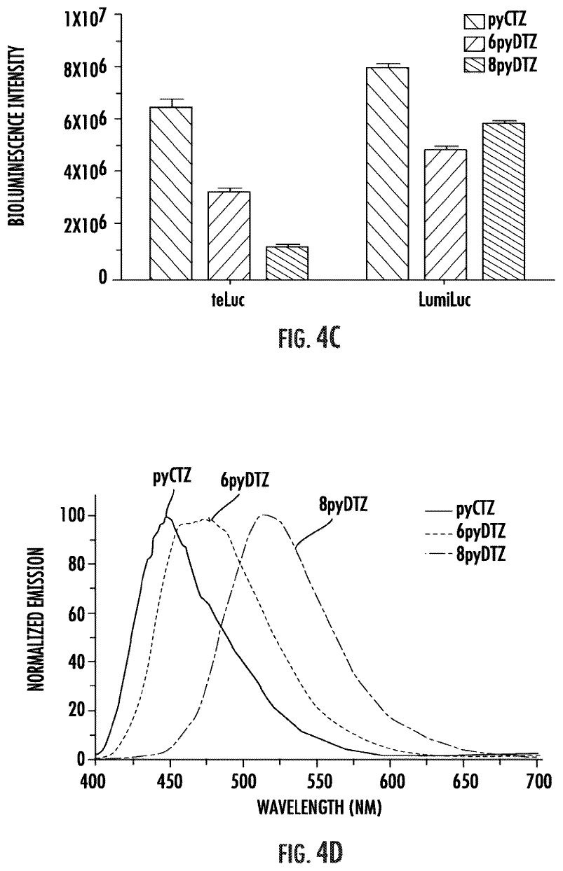

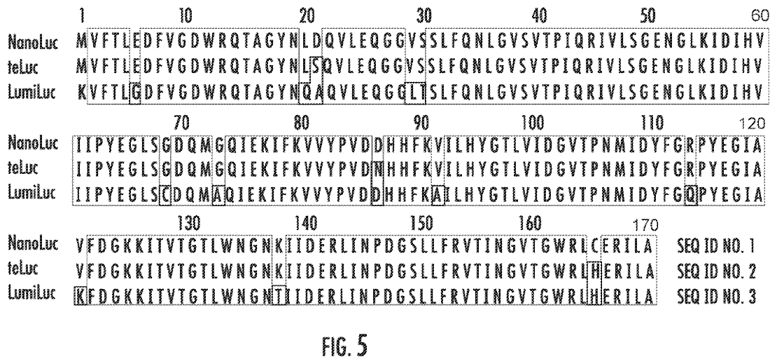

teLuc was previously optimized for DTZ, a substrate with conjugated disubstitutions on the imidazopyrazinone core. 8pyDTZ exhibits about 30 nm red-shift but the emission of teLuc-8pyDTZ has been greatly attenuated compared to teLuc-DTZ. teLuc was then engineered for increased photon flux in the presence of 8pyDTZ. On the basis of a published apo-nanoKAZ structure and our computational model, random mutations were first introduced to residues 20 and 21 close to a putative substrate-binding pocket (FIGS. 4A and 4B). After screening for improved mutants, residues 28, 30, and 31 located deeper in the putative catalytic site were further randomized. From the first two rounds of protein engineering, a teLuc-L20Q/S21A/V29L/S30T mutant was identified, to which random mutations were further introduced using error-prone PCR. After eight additional rounds of mutagenesis and screening, a LumiLuc luciferase was derived with 12 total mutations and about 5-fold enhancement of 8pyDTZ bioluminescence from teLuc (FIGS. 4C and 5).

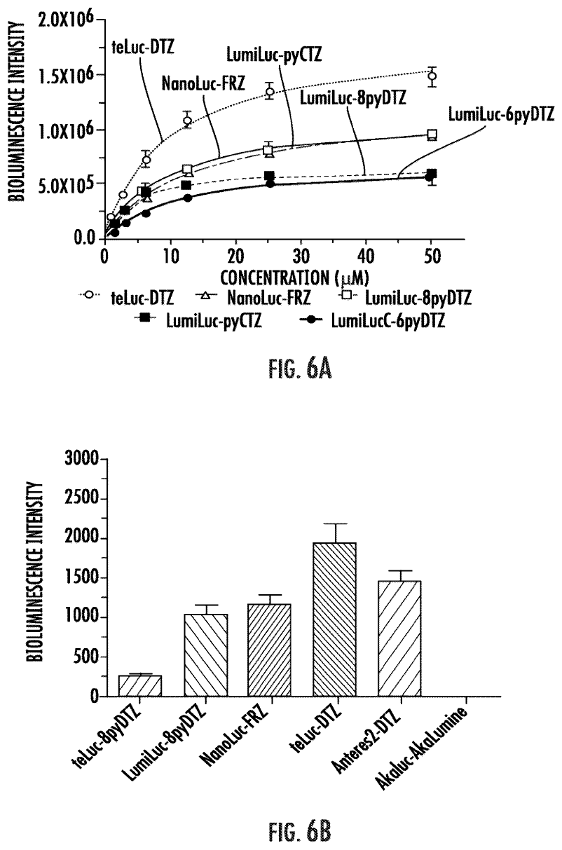

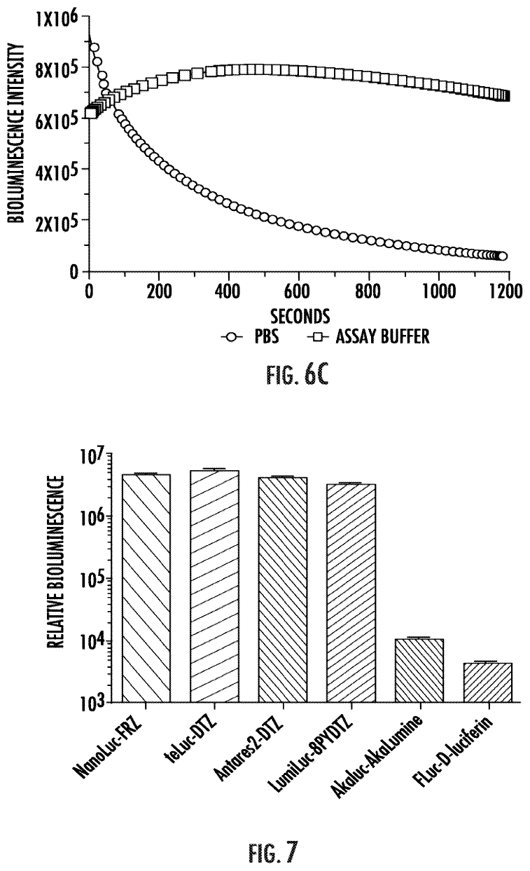

The resultant LumiLuc-8pyDTZ pair has an emission peak at 525 nm. Its in vitro maximal photon emission rate (Vmax) is about 60% and about 36% of NanoLuc-FRZ and teLuc-DTZ, respectively. The apparent Michaelis constant (KM) of LumiLuc-8pyDTZ was 4.6 μM, lower than that of teLuc-DTZ or NanoLuc-FRZ (FIG. 6A). This reduced KM is practically beneficial, since LumiLuc-8pyDTZ would be relatively brighter when effective substrate concentrations are limited, such as in live cells (FIG. 6B) and in vivo. Similar to NanoLuc and teLuc, the bioluminescence kinetics of LumiLuc is flash-type in phosphate buffer saline (PBS) and glow-type in a specially formulated assay buffer (FIG. 6C).

LumiLuc has broad substrate specificity. It improved the photon flux of 3a (pyCTZ) and 3d (6pyDTZ) from teLuc by about 120% and about 150%, respectively (FIG. 4C). The directed evolution process to enhance photon flux of teLuc for 8pyDTZ did not preclude the luciferase from catalyzing other structurally relevant substrates. LumiLuc is capable of efficiently generating blue, teal, or yellow bioluminescence when paired with pyCTZ, 6pyDTZ or 8pyDTZ (λmax: 450, 476, and 525 nm, respectively; FIG. 4D), thereby leading to a new family of ATP-independent bioluminescent reporters with water-soluble substrates.

### Example 3: LumiLuc-8pyDTZ in Cultured Mammalian Cells

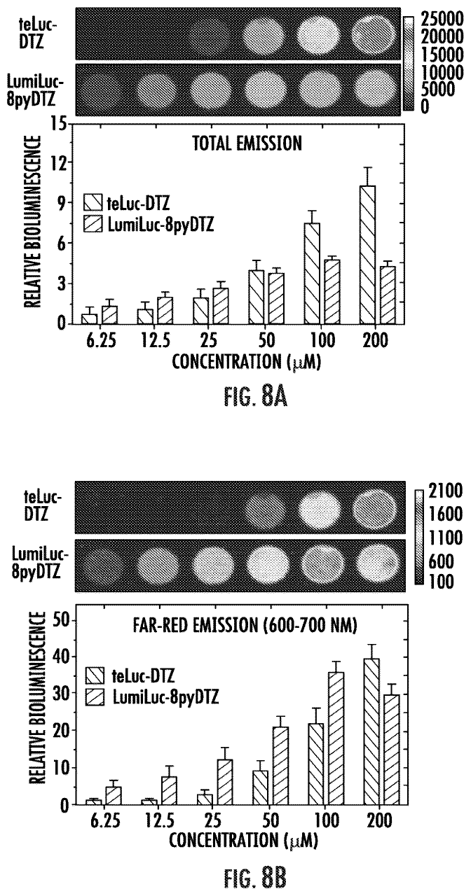

Next, LumiLuc-8pyDTZ was evaluated in human embryonic kidney (HEK) 293T cells transiently expressing the luciferase (FIG. 7). The LumiLuc-8pyDTZ pair produced about 3- to 5-fold more bioluminescence than teLuc-8pyDTZ at all tested substrate concentrations. Moreover, despite that LumiLuc-8pyDTZ is less bright than teLuc-DTZ at saturated substrate concentrations, LumiLuc-8pyDTZ is notably brighter than teLuc-DTZ at low substrate concentrations (from 6.25 to 25 μM; FIG. 8A). Far-red emission at wavelengths longer than 600 nm is more indicative of the in vivo performance of bioluminescent reporters, because mammalian tissue is more transparent in this spectral region. To compare far-red emission intensities of bioluminescent reporters, HEK 293T cells were imaged in the presence of a 600-700 nm bandpass filter. At substrate concentrations from 6.25 to 100 μM, LumiLuc-8pyDTZ consistently produces 1.6- to 3.9-fold higher photon flux than teLuc-DTZ (FIG. 8B).

ATP-dependent luciferases, such as FLuc and Akaluc, consume one ATP molecule in each catalytic cycle, leading to metabolic disruption. Instead, ATP-independent LumiLuc does not use ATP for catalysis. ATP/ADP ratios were monitored in live HEK 293T cells using PercevalHR, a previously reported fluorescent ATP/ADP biosensor. No ATP perturbation was observed from 8pyDTZ-treated, LumiLuc-expressing cells.

### Example 4: LumiLuc-8pyDTZ to Track Tumor Growth in a Mouse Xenograft Model

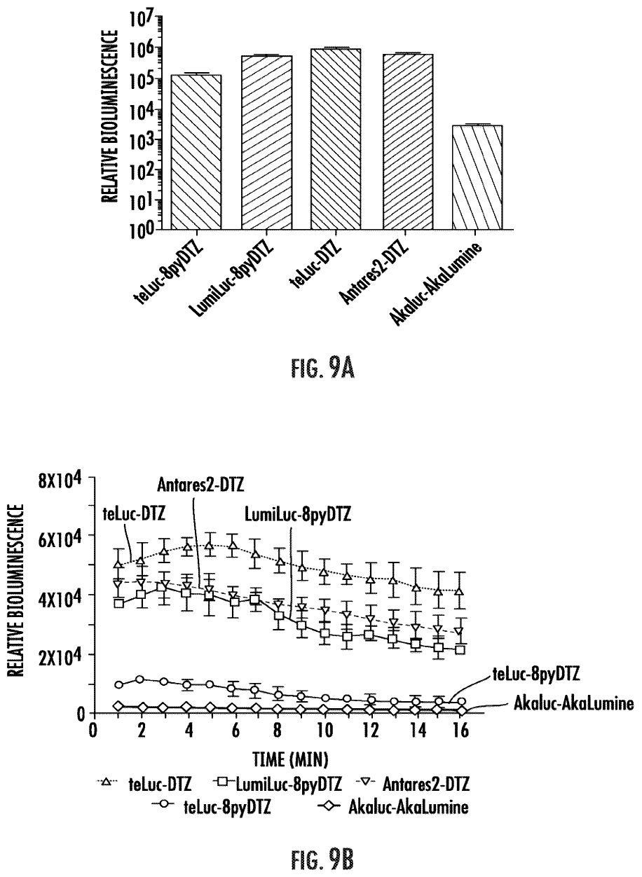

Bioluminescence imaging (BLI) has been a popular imaging modality for various animal models. The recently reported Akaluc-AkaLumine and Antares2-DTZ pairs are two benchmark reporters for in vivo BLI. A biologically-relevant tumor xenograft mouse model was adapted to compare these bioluminescent reporters. Cervical cancer HeLa cell lines stably expressing individual luciferases were generated, including teLuc, Antares2, LumiLuc, and Akaluc (FIGS. 9A and 9B). Next, 10⁴ or 105 luciferase-expressing HeLa cells were injected into the left or right dorsolateral trapezius and thoracolumbar regions of immunodeficient NU/J mice (day 0) and monitored tumor growth over 4 weeks. Bioluminescence was quantified on days 1, 3, 5, 7, 14, and 28 after tail vein injection of corresponding substrates. AkaLumine-HCl was delivered at a dose of 1.5 μmol per mouse. This dosage (about 75 nmol/g), which is normalized against the body weights of mice, is identical to the previously reported dosage. Moreover, when 3 μmol of AkaLumine-HCl per mouse (about 150 nmol/g) was used, death for 2 out of 3 mice was observed in the pilot experiment. 8pyDTZ was dissolved in normal saline to its saturation concentration and intravenously injected, resulting in a dose of 0.2 μmol per mouse (about 10 nmol/g).

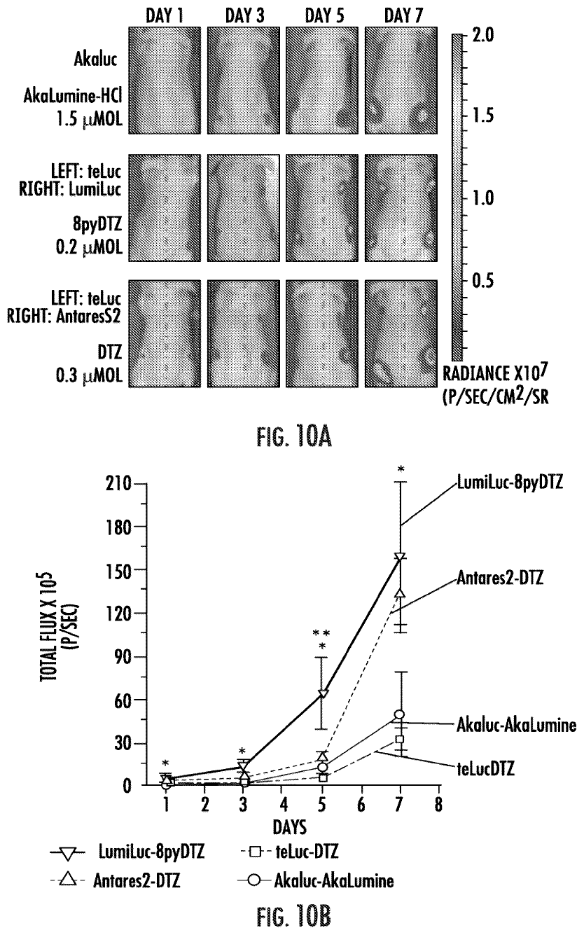

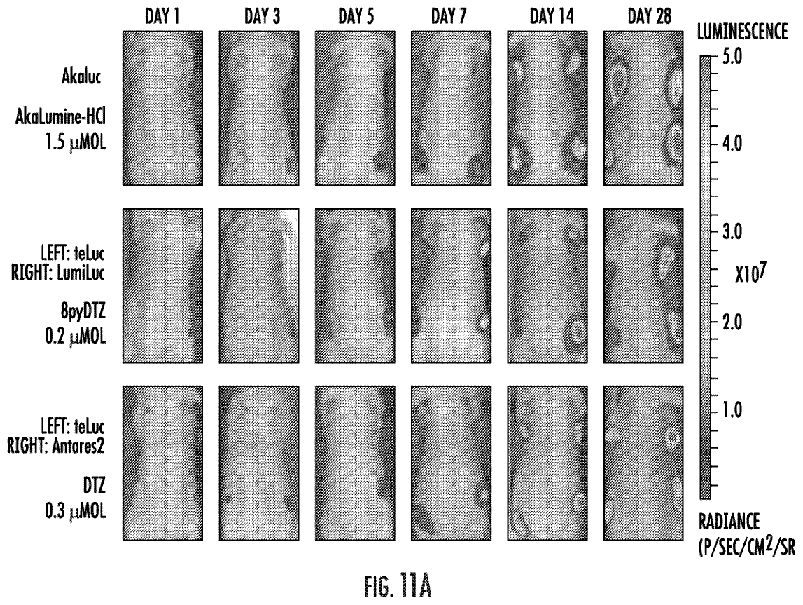

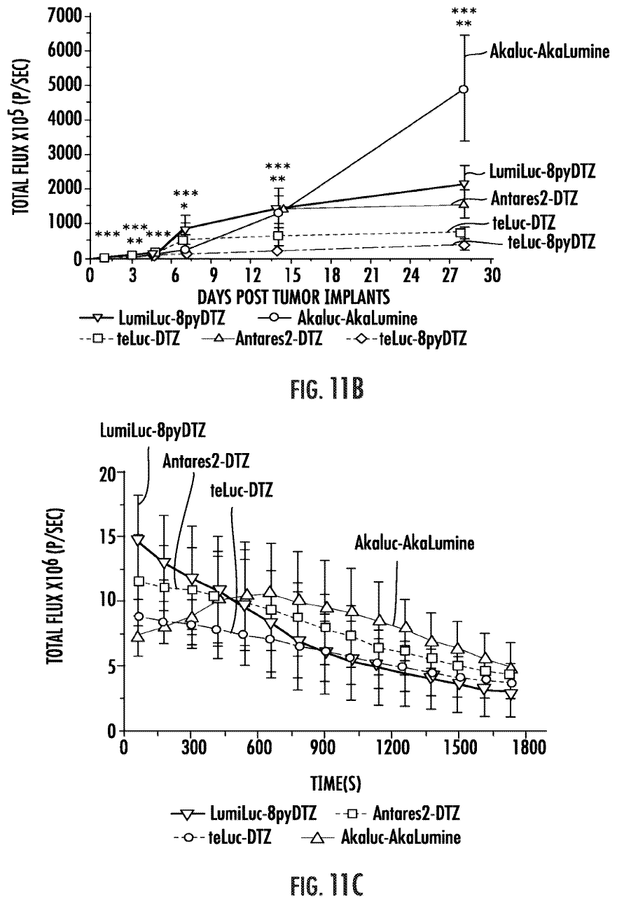

The LumiLuc-8pyDTZ pair showed detectable bioluminescence on day 1 at sites inoculated with 10⁴ cells, and kept exhibiting about 3-fold higher photon flux over Akaluc-AkaLumine up to day 7 (FIGS. 10A and 10B). The signals for Akaluc-AkaLumine at sites inoculated with 10⁴ cells were not consistently higher than background until day 3. Furthermore, the in vivo brightness of LumiLuc-8pyDTZ is comparable to, if not higher than, the Antares2-DTZ pair (FIGS. 10A, 10B and 11A-11C), despite the fact that the majority of emitted photons from LumiLuc-8pyDTZ has not yet exceeded 600 nm. These data collectively support that LumiLuc-8pyDTZ is a superior bioluminescent reporter system for high-sensitivity in vivo BLI.

The bioluminescence of Akaluc-AkaLumine eventually surpassed LumiLuc-8pyDTZ from day 14 (FIGS. 11A-11C). In addition to differences in biodistribution and pharmacokinetic properties of AkaLumine and 8pyDTZ, it was interpreted that 8pyDTZ may be a limiting reagent in large tumors because AkaLumine-HCl could be delivered into mice at a much higher dose than 8pyDTZ due to the higher solubility of AkaLumine-HCl. It may be possible to further enhance the in vivo performance of marine luciferases and their derivatives by further increasing the water solubility and thus the administration dosage of CTZ and DTZ analogs.

This data provides for the monitoring of the disclosed luciferase-luciferin pairs for in vivo monitoring of tumor models. For example, bioluminescence can be monitored in a tumor model by establishing a luciferase expressing cell (e.g., a cell expressing LumiLuc, teLuc, RLuc8 and OpyLuc) in the model, e.g., by transfecting cells in the model and/or by administering to that model already transfected cells expressing a luciferase, and administering to the model a luciferin (e.g., pyCTZ, pyOHCTZ, pyOMeCTZ, pyOEtCTZ, pyiPrCTZ, 2pyDTZ, 6pyDTZ, 60pyDTZ or 8pyDTZ). Using these luciferase-luciferin pairs as reporter systems the bioluminescence can be used as a tool to monitor the tumor.

### Example 5: Engineering of Bret-Based Lumiscarlet and Tescarlet for Deep-Tissue BLI

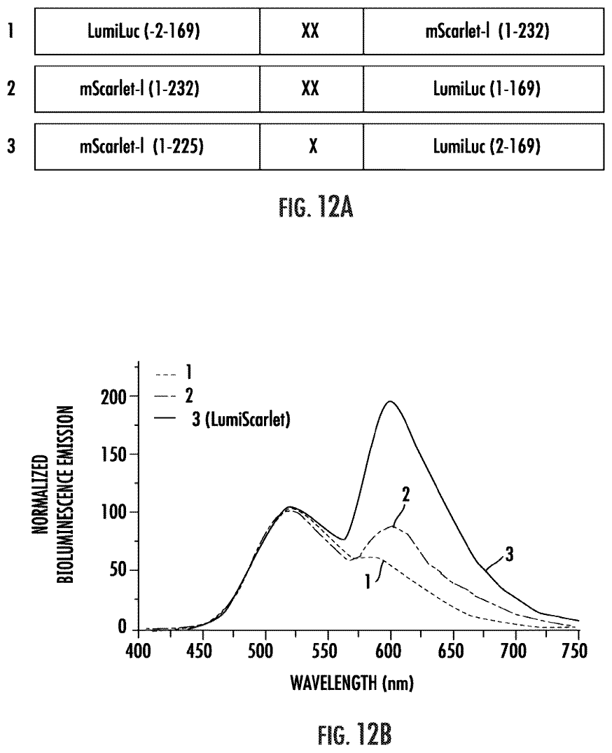

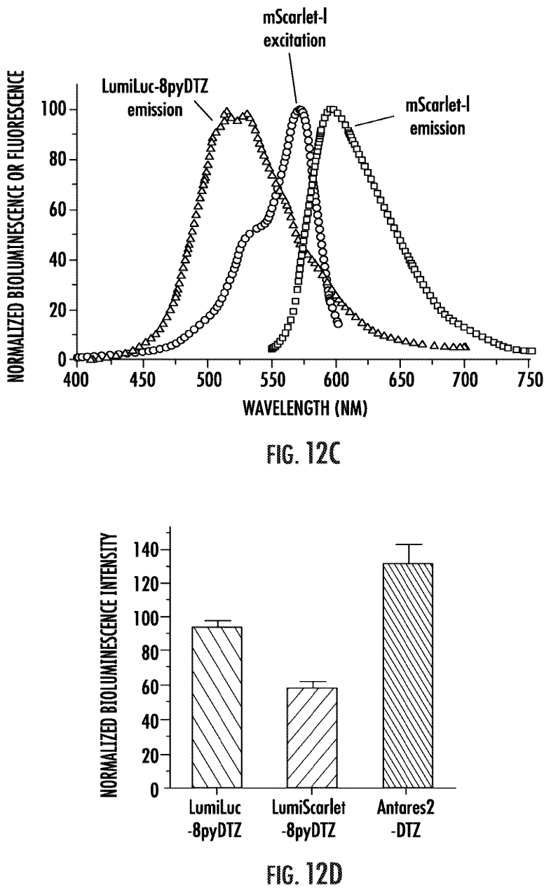

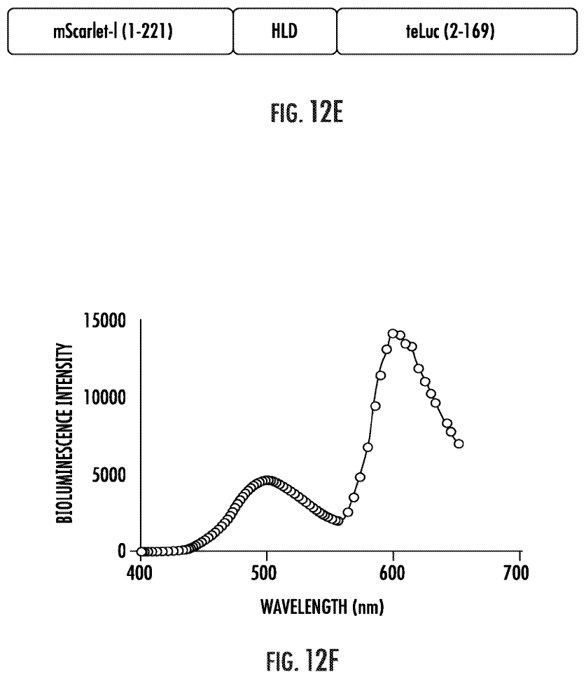

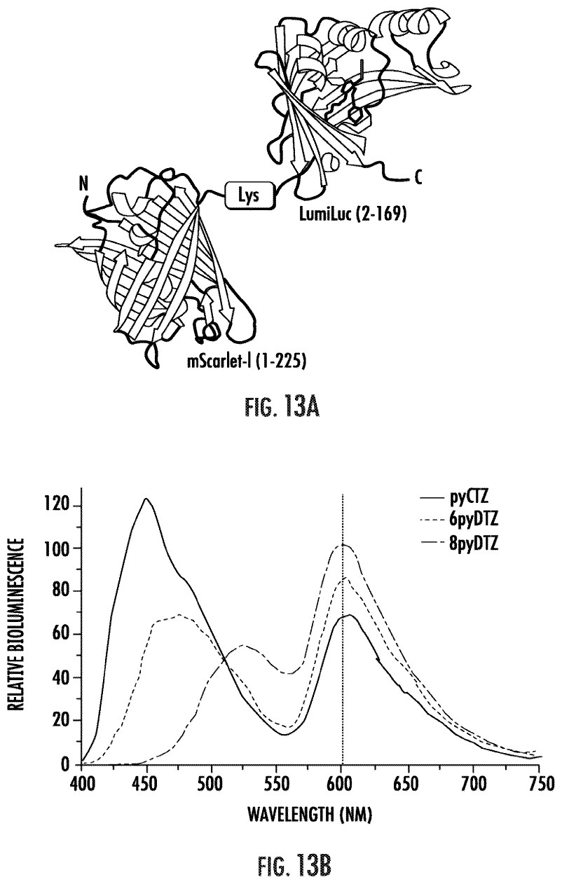

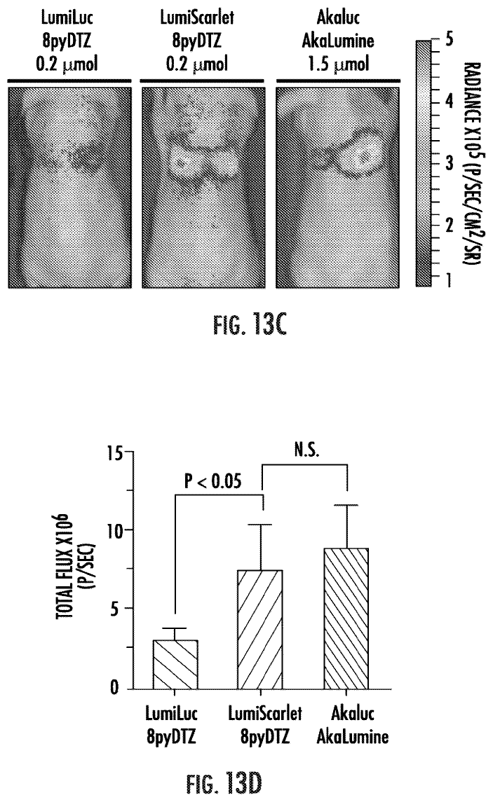

mScarlet-I is a recently reported red fluorescent protein with high quantum yield and excellent performance as a Förster resonance energy transfer (FRET) acceptor. It was thus hypothesized that LumiLuc could be genetically fused to mScarlet-I for BRET, thereby red-shifting the emission of LumiLuc. Several fusion strategies between LumiLuc and mScarlet-I were explored, libraries were constructed by randomizing the linkers, and mutants with high BRET efficiency were screened (FIGS. 12A and 12B). A mutant was identified, namely LumiScarlet (FIG. 13A), which is a fusion protein of mScarlet-I (residues 1-225; Bindels et al, 2016, Nature Methods, 14 (1), 53-56; incorporated herein by reference) linked to the N-terminus of LumiLuc (residues 2-169; SEQ ID NO. 3) through a single-residue “Lys” linker (nucleotide and polypeptide sequences provided herein as SEQ ID NOs. 4 and 5, respectively).

High BRET efficiency was achieved with LumiScarlet in the presence of either pyCTZ, or 6pyDTZ, or 8pyDTZ (FIG. 13B). In particular, since the emission spectrum of LumiLuc-8pyDTZ overlaps well with the excitation spectrum of mScarlet-I (FIG. 12C), about 51% of the total emission of LumiScarlet, when paired with 8pyDTZ, was longer than 600 nm (Table 3).

TABLE 3
BRET-BASED BIOLUMINESCENT REPORTERS THAT ARE BASED ON
NANOLUC AND ITS DERIVATES.

| | | | Size | λₘₐₓ | | Photon > 600 nm |
|---|---|---|---|---|---|---|
| BRET Construct | BRET Donor | BRET Acceptor | (kDa) | (nm) | Luciferin | (%) |
| Lumi Scarlet | LumiLuc | mScarlet-I | 44 | 527, | 8pyDTZ | 51 |
| | | | | 600 | | |
| LumiLuc | mScarlet-I | | 44 | 476, | 6pyDTZ | 38 |
| | | | | 600 | | |
| LumiLuc | mScarlet-I | | 44 | 450, | pyCTZ | 26 |
| | | | | 600 | | |
| Antares | NanoLuc | CyOFP | 70 | 456, | FRZ | 23 |
| | | | | 583 | | |
| Antares2 | teLuc | CyOFP | 70 | 501, | DTZ | 33 |
| | | | | 583 | | |
| ReNL | NanoLuc | tdTomato | 72 | 459, | FRZ | 24 |
| | | | | 583 | | |

Next, the newly engineered LumiLuc-8pyDTZ and LumiScarlet-8pyDTZ were compared with Akaluc-AkaLumine for deep-tissue BLI. A million Hela cells stably expressing corresponding luciferases were injected into each of NU/J mice via tail vein and performed BL imaging 4 h later. Immunodeficient mice were used here to minimize immune responses to Hela cells, so that signals will be mostly from live cells trapped in the lungs. LumiScarlet gave about 3-fold higher detectable signals than LumiLuc under this condition (FIGS. 13C and 13D), even though the in vitro brightness of LumiScarlet is only about 70% of LumiLuc (FIG. 12D). Moreover, the signals from LumiScarlet-8pyDTZ were comparable to the signals from Akaluc-AkaLumine.

Of note, some diffuse signals were observed from areas other than the lungs. These signals were not caused by substrate background, as injection of 8pyDTZ into blank mice resulted in only weak background much lower than what was observed in FIG. 13C. Luciferase activities were detectable in blood after tail vein injection of luciferase-expressing Hela cells, suggesting partial lysis of luciferase-labeled cells during cell injection. Also, in contrast to ATP-independent LumiLuc and LumiScarlet, ATP-dependent Akaluc is enzymatically inactive in serum because of relatively low ATP levels and further deactivation by serum components. Thus, diffuse signals from LumiScarlet in FIG. 13C were likely from the LumiScarlet luciferase released into blood.

Collectively, the deep-tissue BLI results confirm that red-shifted BRET-based LumiScarlet has better mammalian tissue penetration than LumiLuc. Moreover, LumiScarlet-8pyDTZ is a novel, ATP-independent bioluminescent reporter with exceptional deep-tissue BLI performance comparable to ATP-dependent Akaluc-AkaLumine.

Similar to the BRET-based strategy of creating LumiScarlet, a BRET-based fusion of teLuc and mScarlet-I was engineered. Libraries were constructed to randomize the linker between teLuc and mScarlet-I. A mutant was identified, namely teScarlet (FIG. 12E), which is a fusion protein of mScarlet-I (residues 1-221) linked to the N-terminus of teLuc (residues 2-169) through a three-residue “His-Leu-Asp” linker (nucleotide and polypeptide sequences provided herein as SEQ ID NOs. 7 and 8, respectively). High BRET efficiency was also achieved with teScarlet in the presence of DTZ (FIG. 12F).

### Discussion of Examples 1-5

Conventionally, ATP-dependent bioluminescent reporters, such as FLuc and Akaluc, are considered to be more useful for in vivo BLI than ATP-independent marine luciferases, because the emission of ATP-dependent insect luciferases is often at the red end of the visible spectrum where the mammalian tissue is relatively transparent. However, these insect luciferases require ATP and Mg²⁺ for bioluminescence. The ATP- and Mg²⁺-dependency is sometimes problematic because ATP and Mg²⁺ levels may vary under different biological circumstances. In particular, ATP-dependent luciferases are inactive in extracellular space and common biological fluids such as blood and urine, where ATP accessibility is limited. Moreover, ATP-dependent luciferases consume ATP in bioluminescence reactions and may cause concerns such as metabolic disruption. In contrast, most ATP-independent marine luciferases are enzymatically active in extracellular space and common biological fluids; they do not consume ATP for bioluminescence. Furthermore, some marine luciferase derivatives have fast catalytic turnover and thus give high photon flux. It is therefore not surprising that marine luciferase and their derivatives, such as NanoLuc and Gaussia luciferase, have been widely used for in vitro bioluminescence assays. However, currently, the in vivo applications of marine luciferases are hindered by their blue emission and poor substrate water solubility. As disclosed herein, combined chemical synthesis and protein engineering approaches yielded enhanced ATP-independent marine luciferases for in vivo BLI by developing red-shifted colors and water-soluble substrates.

First, a series of pyridyl CTZ and DTZ analogs with diverse emission profiles were prepared. The water solubility of these synthetic analogs generally increased by about 10-fold from their ancestors. These substrate analogs can not only be paired with the new luciferases engineered here, but also existing ATP-independent reporters, such as RLuc and aequorin.

Further, a luciferase was engineered for the 8pyDTZ substrate via directed protein evolution. The resultant LumiLuc-8pyDTZ bioluminescent reporter system exhibited reduced KM and red-shifted emission. These factors favored in vivo BLI. As a result, LumiLuc-8pyDTZ showed high sensitivity in a mouse xenograft model. In addition, LumiLuc-8pyDTZ did not perturb the intracellular ATP/ADP level, and 8pyDTZ could be dissolved up to about 2 mM in low-viscosity saline without using irritative and toxic organic cosolvent. Therefore, the efforts disclosed herein enhanced not only the biocompatibility of bioluminescent reporters, but also reproducibility for intravenous injections.

Furthermore, a BRET-based LumiScarlet reporter was developed for further red-shifted emission. The emission of LumiLuc-8pyDTZ overlaps well with the excitation of mScarlet-I, an excellent red-emitting resonance energy transfer acceptor. LumiScarlet-8pyDTZ exhibited high brightness, significant emission longer than 600 nm, and excellent tissue penetration. LumiScarlet-8pyDTZ was comparable to NIR-emitting Akaluc-AkaLumine in a mouse model for deep-tissue BLI. Moreover, because LumiScarlet is enzymatically active in blood, it will be an excellent reporter for monitoring targets of interest in the blood of in vivo models.

LumiLuc is a luciferase with broad substrate specificity. When it was paired with different substrates, intense blue, teal, and yellow bioluminescence was generated. Subsequently, different emission profiles from LumiScarlet were gained in the presence of different substrates. The use of LumiScarlet-8pyDTZ for deep-tissue imaging was also demonstrated. In addition, because the two emission peaks of LumiScarlet-pyCTZ or LumiScarlet-6pyDTZ are more separated than LumiScarlet-8pyDTZ, LumiScarlet-pyCTZ and LumiScarlet-6pyDTZ will be useful for studying protein-protein interactions or constructing BRET-based biosensors.

In summary, disclosed herein are several engineered luciferase-luciferin pairs that emit photons spanning an appreciable range in the visible spectrum. The discoveries disclosed herein greatly enhance the biocompatibility and sensitivity of ATP-independent bioluminescent reporters for in vivo BLI. Future studies are likely to continuously increase the water-solubility of CTZ and DTZ analogs and red-shift the emission of marine luciferases. Subsequently, it is expected that a large array of bioluminescent biosensors will be developed on the basis of these bright, ATP-independent bioluminescent reporters. The new reporters and biosensors will further ease non-invasive imaging of freely moving animals, leading to new biological insights.

### Materials and Methods for Examples 6-9

### Materials and General Methods

Synthetic DNA oligonucleotides were purchased from Integrated DNA Technologies. Restriction endonucleases were purchased from Thermo Fisher Scientific. Q5 high-fidelity DNA polymerase and Taq DNA polymerase were purchased from NEB. Products of PCR and restriction digestion were purified by gel electrophoresis and Syd Laboratories Gel Extraction columns. Plasmid DNA was purified using Syd Laboratories Miniprep columns. DNA sequences were analyzed by Eurofins. AkaLumine-HCl was purchased from Aobious. All other chemicals were purchased from Sigma-Aldrich, Fisher Scientific, or VWR and used without further purification. Bruker Avance DRX 600 and Varian NMRS 600 at the UVA Biomolecular Magnetic Resonance Facility was used to record all NMR spectra. Chemical shift (8) is given in parts per million relative to 1H (7.24 ppm) and 13C (77.23 ppm) for CDCl₃; 1H (2.50 ppm) and 13C (39.5 ppm) for DMSO-d₆; 1H (3.31 ppm) and 13C (49.15 ppm) for methanol-d₄. Splitting patterns are reported as s (singlet), bs (broad singlet), d (doublet), t (triplet), dd (doublet of doublets), and m (multiplet). Coupling constant (J) is given in Hz. High resolution ESI-MS was run on an Agilent 6545 Q-TOF LC/MS system by direct infusion. A Waters Delta Prep ZQ 2000 LC-MS Purification System equipped with a XBridge BEH Amide OBD Prep Column (130 Å, 5 μm, 30 mm×150 mm) was used for preparative reverse-phase HPLC purifications. Images were analyzed using the Fiji image analysis software. Microsoft Excel and GraphPad Prism were used to analyze data and prepare figures.

### Chemical Synthesis

### 3-benzyl-5-(4-methoxyphenyl) pyrazin-2-amine (2)

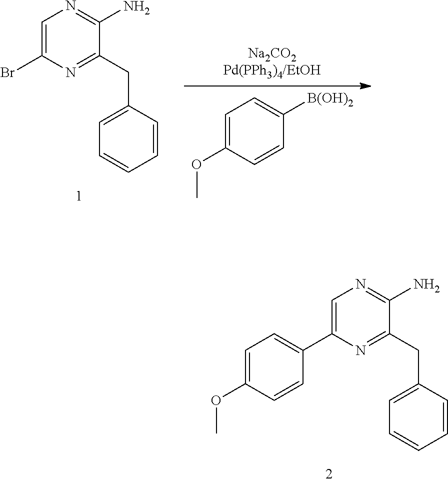

1 was prepared following the published synthesis methods. To a solution of Pd (PPh₃) 4 (230 mg, 0.2 mmol, 0.1 equiv.) in 50 mL EtOH was added 1 (528 mg, 2 mmol, 1 equiv.), 1N Na₂CO3 solution (4 mL, 4 mmol, 2 equiv.) and 4-methoxylphenylboronic acid (304 mg, 2 mmol, 1 equiv.). The resultant mixture was stirred at 80° C. under argon for 12 h. The solvent was removed in vacuo and the residue was suspended in 100 mL ddH₂O, which was extracted twice with EtOAc (100 mL). The organic layers were combined and dried over anhydrous Na₂SO₄, filtered and removed in vacuo. The residue was purified by silica column chromatography with elution (Ethyl acetate:Hexane=1:1) to yield compound 2 as yellow solid (413 mg, 71%). ¹H NMR (600 MHz, DMSO-d₆) δ 8.33 (s, 1H), 7.82 (d, J=8.8 Hz, 2H), 7.32 (d, J=6.7 Hz, 2H), 7.26 (t, J=7.6 Hz, 2H), 7.17 (t, J=7.4 Hz, 1H), 6.95 (d, J=8.8 Hz, 2H), 6.26 (s, 2H), 4.05 (s, 2H), 3.76 (s, 3H). ¹³C NMR (150 MHz, DMSO-d₆) δ 158.9, 152.2, 139.8, 139.0, 138.2, 136.2, 128.9, 128.2, 126.1, 126.1, 114.1, 55.1, 38.6. HRMS (ESI-TOF) calcd for C₁₈H₁₇N₃O [M+H]⁺: 292.1372. found: m/z 292.1369.

### 8-benzyl-6-(4-methoxyphenyl)-2-(pyridin-4-ylmethyl)imidazo[1,2-a]pyrazin-3 (7H)-one (pyQMeCTZ)

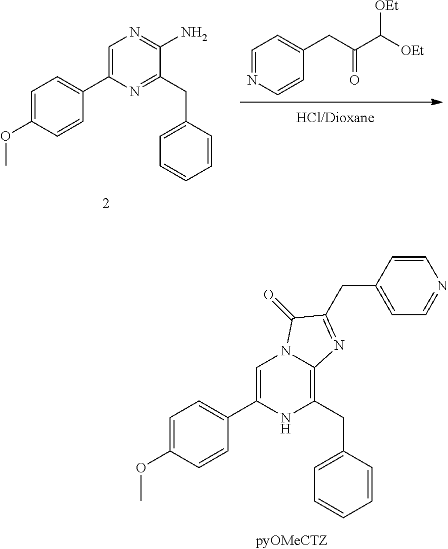 [SMILES: COc1ccc(-c2cnc(N)c(Cc3ccccc3)n2)cc1 ; CCOC(OCC)C(=O)Cc1ccncc1 ; COc1ccc(-c2cn3c(=O)c(Cc4ccncc4)nc-3c(Cc3ccccc3)[nH]2)cc1]

To a solution of 2 (29 mg, 0.1 mmol, 1 equiv.) and 3-pyridin-4-yl-1,1-diethoxyacetone (45 mg, 0.2 mmol, 2 equiv.) in 2 mL degassed 1,4-dioxane was added 1 mL 6N HCl. The resulting mixture was stirred at 80° C. in a sealed tube for 12 h. The solvent was then removed in vacuo and the residue was dissolved in 1 mL (ACN:H2O=1:1) and next purified with preparative RP-HPLC (acetonitrile/water=1:99 to 90:10, 20 mL/min, UV 254 nm). Product fractions were combined and lyophilized to give pyOMeCTZ as yellow powder (10 mg, 24%), which has to be stored as solid at −80° C. for long-term stability. ¹H NMR (600 MHz, Methanol-d₄) δ 8.82 (d, J=6.4 Hz, 2H), 8.27 (s, 1H), 8.09 (d, J=6.2 Hz, 2H), 7.73 (d, J=8.7 Hz, 2H), 7.43 (d, J=7.5 Hz, 2H), 7.32 (t, J=7.6 Hz, 2H), 7.26 (t, J=7.2 Hz, 1H), 7.10 (d, J=8.7 Hz, 2H), 4.66 (s, 2H), 4.61 (s, 2H), 3.87 (s, 3H). ¹³C NMR (150 MHz, Methanol-d₄) δ 142.7, 137.0, 130.2, 130.1, 129.1, 128.7, 126.5, 115.9, 56.2. HRMS (ESI-TOF) calcd for C₂₆H₂₂N₄O₂ [M+H]⁺: 423.1743. found: m/z 423.1740.

### 2-benzyl-6-phenyl-8-(pyridin-4-yl)imidazo[1,2-a]pyrazin-3 (7H)-one (pyDTZ)

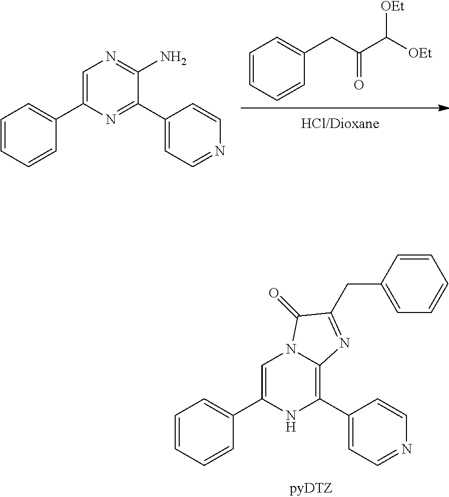 [SMILES: Nc1ncc(-c2ccccc2)nc1-c1ccncc1 ; CCOC(OCC)C(=O)Cc1ccccc1 ; O=c1c(Cc2ccccc2)nc2c(-c3ccncc3)[nH]c(-c3ccccc3)cn1-2]

To a solution of 5-phenyl-3-(pyridin-4-yl) pyrazin-2-amine (25 mg, 0.1 mmol, 1 equiv.) and 1,1-diethoxy-3-phenylpropan-2-one (89 mg, 0.4 mmol, 4 equiv.) in 5 mL degassed 1,4-dioxane was added 0.8 mL 6 N HCl, and the resulting mixture was stirred at 80° C. in a sealed tube for 12 h. The solvent was then removed in vacuo and the residue was dissolved in 1 mL (ACN:H2O=1:1) and next purified with preparative RP-HPLC (acetonitrile/water=1:99 to 90:10, 20 mL/min, UV 254 nm). Product fractions were combined and lyophilized to give pyDTZ as brown powder (8 mg, 22%). ¹H NMR (600 MHz, Acetonitrile-d₃ and D₂O, ratio=9:1) δ 9.31 (d, J=6.8 Hz, 2H), 8.84 (d, J=6.8 Hz, 2H), 8.54 (s, 1H), 8.09 (d, J=8.0 Hz, 2H), 7.52 (t, J=7.6 Hz, 2H), 7.44 (t, J=7.3 Hz, 1H), 7.35 (d, J=7.7 Hz, 2H), 7.28 (t, J=7.7 Hz, 2H), 7.24-7.21 (m, 1H), 4.19 (s, 2H). ¹³C NMR (150 MHz, Methanol-d₄) δ 143.0, 140.7, 140.1, 139.5, 137.5, 132.0, 130.2, 129.9, 129.6, 129.4, 127.8, 127.5, 127.4, 126.5, 113.8, 33.6. HRMS (ESI-TOF) calcd for C₂₄H₁₈N₄O [M+H]⁺: 379.1481. found: m/z 379.1477.

### Plasmid Construction

Polymerase chain reactions with various synthetic oligonucleotide pairs (see Table 4) were used to amplify genetic elements. Generating gene libraries with randomizations were previously described. The above-mentioned screening approach was applied to the selection process of random mutagenesis by error-prone PCR. Oligonucleotides pBAD-F and pBAD-R were used to create a library with randomization by using Taq DNA polymerase, 0.2 mM MnCl2, and unbalanced dNTPs to promote amplification errors. The PCR product was digested with Xho I and Hind III restriction enzymes and ligated into a predigested, compatible pBAD/His B plasmid. To create mammalian expression plasmids containing the NFKB response element, NFKB_SacI F and NFKB_BgIII_R were used to amplify the fragment from pHAGE NFKB-TA-LUC-UBC-GFP-W plasmid (Addgene: 49343), which was further treated with Sac I and BgI II restriction enzymes and ligated into a predigested, compatible SRE reporter vector_559 plasmid (Addgene: 82686). For the antioxidant response element, the DNA fragment was synthesized by IDT and ligated into Sac I and BgI II predigested SRE reporter vector_559 plasmid. The OpyLuc, RLuc8, and Akaluc genes were cloned into corresponding plasmids containing the desired response element by using opyluc_AscI_Kozak_F/opyluc_FseI_R, Rluc_AscI_Kozak_F/Rluc_Fsel_R, or Akaluc_Ascl_Kozak_F/Akaluc_FseI_R oligonucleotide pairs with Asc I and Fse I double digestion. All ligation products were used to transform Escherichia coli DH10B electrocompetent cells, which were next plated on LB agar plates supplemented with ampicillin (100 μg/mL).

TABLE 4
OLIGONUCLEOTIDES USED IN THIS STUDY.

| | SEQ ID | |
|---|---|---|
| Oligo name | NO: | Nucleotide sequence (5′→3′) |
| pBAD-F | 19 | ATGCCATAGCATTTTTATCC |
| pBAD-R | 20 | GATTTAATCTGTATCAGG |
| NFkB_SacI_F | 21 | TACCGAGCTCATCCAGTTTGGACTAGTGG |
| NFKB_BgIII_R | 22 | AGCCCAGATCTCCTCTAGAGTCTAGATCTGG |
| opyluc_AscI_Kozak_F | 23 | AAAGCCACCGGCGCGCCGCCGCCACCATGGTCTTCAC |
| opyluc_FseI_R | 24 | TCTCGAGATTTGTTCGAAGCGGCCGGCCTTACG |
| | | CAGAGATGCGTTCATGCA |
| Akaluc_AscI_Kozak_F | 25 | AAAGCCACCGGCGCGCCGCCGCCACCATGGAAGATG |
| | | CCAAAAACATTAAGA |
| Akaluc_FseI_R | 26 | TCGAAGCGGCCGGCCTTACACGGCGATCTTGCCGTCC |
| | | TTCTT |
| Rluc_AscI_Kozak_F | 27 | AAAGCCACCGGCGCGCCGCCGCCACCATGGCTTCCAA |
| | | GGTGTACGACC |
| Rluc_FseI_R | 28 | TCGAAGCGGCCGGCCTTACTGCTCGTTCTTCAGCACG |
| | | CGCT |

### Preparation of Mammalian Cell Culture and Cell Lysate

HEK 293T cells were cultured and transfected as previously described. The number and density of cells in Dulbecco's phosphate-buffered saline (DPBS) were determined using a hemocytometer. Cells were next diluted in DPBS to gain the desired numbers in each 50 μL solution. Cell lysates were obtained by incubating the desired number of cells in a CelLytic M solution for 15 minutes and centrifuged.

### Library Screening

DH10B cells containing luciferase mutants were plated on LB agar plates supplemented with ampicillin (100 μg/mL) and L-arabinose (0.02%, w/v) and incubated at 37° C. overnight to form bacterial colonies. Agar plates were left at room temperature for another 6 h, and this was followed by bioluminescence imaging using a luminescence dark box (UVP Bio Spectrum) equipped with a QSI 628 cooled CCD camera (Quantum Scientific Imaging). Digital images were acquired after spraying about 200 μL of 50 μM pyDTZ to each agar plate, and next, images were processed with the Fiji image analysis software to derive bioluminescence intensities of individual colonies. For each round of selection, colonies showing bright bioluminescence were chosen and inoculated in 1 mL liquid LB broth containing ampicillin (100 μg/mL) and L-arabinose (0.02%, w/v) in 96-well deep plates. After overnight growth at 37° C. and 250 rpm, the cultures were moved onto a shaker at room temperature for another 6 h. The 96-well plates were centrifuged and the pellet in each well was lysed with 200 μL B-PER. After 30-minute incubation, the 96-well plates were centrifuged again. Next, 2 μL lysate from each sample was transferred to the wells of new white 96-well plates where 100 μL of 20 μM pyDTZ in assay buffer was added to each well. Bioluminescence activities of individual samples were measured on a microplate reader. Kinetics were followed for 0.1 s signal integration every 30 s for a total of 10 min. Meanwhile, 2 μL lysate from each sample was added to 100 μL of 20 μM pyOMeCTZ in assay buffer. The selectivity was determined by the specific activity toward pyDTZ/activity toward pyOMeCTZ. Top three mutants showing exceptionally high bioluminescence selectivity of pyDTZ over pyOMeCTZ were chosen for next-round selection, sequencing, and other additional characterization.

### In Vitro Bioluminescence Characterization

Luciferases were expressed and purified as previously described. A microplate reader was used for all in vitro bioluminescence characterizations. To record bioluminescence emission spectra, 50 μL of luciferin substrates was injected into the wells of white 96-well plates containing 50 μL of pure enzymes in PBS (1.5 mM ATP and 5 mM MgSO₄ were supplemented for Akaluc). Kinetic measurements were taken every 30 s post injection with 0.1 s integration and 10 s shaking during intervals. To derive values for apparent Michaelis constants (Kₘ), substrate concentrations varied from 0.78 to 50 μM, and 10-min integrated bioluminescence at individual substrate concentrations were used to fit the Michaelis-Menten equation.

Bioluminescence Imaging with Luminescence Dark Box

UVP Bio Spectrum luminescence dark box was used for all bioluminescence imaging. To record bioluminescence imaging with pure enzymes, 50 L of 60 μM substrates were injected into corresponding 50 μL pure enzymes (final concentration: 10 nM for RLuc8 and OpyLuc; 100 nM for Akaluc), and the bioluminescence imaging was collected with 10 s exposure time. A filter wheel equipped with a Chroma Blue 360-500 nm, a Chroma Green 495-580 nm, and a Chroma Red 600-700 nm filter was used to acquire emission in each channel. To use the luminescence dark box to directly image cells, we added luciferase-expressing HEK 293T cells (5,000 cells per well for RLuc8 and OpyLuc; 30,000 cells per well for Akaluc) and the indicated luciferin substrates solution were injected into wells of a white 96-well plate. Final concentration of each substrate were 25 μM for pyOMeCTZ, 10 μM for pyDTZ, and 100 μM for AkaLumine-HCl. Bioluminescence was imaged in the luminescence dark box immediately after substrate addition. The camera exposure time was set at 30 s. All images were analyzed using the Fiji image analysis software.

### Transfection and Activation of Signaling Pathways in HEK293T Cell Line

HEK293T cells were transfected at about 70% confluency by using plasmid DNA:PEI=3:9 mixture. Plasmids used in this study included SRE-RLuc8, ARE-OpyLuc, and NF-KB-Akaluc, NF-KB-RLuc8, SRE-OpyLuc, ARE-Akaluc and CMV-Akaluc. 3 h after transfection, the medium was removed and replaced by fresh medium. The cells were allowed to recover for another 3 h. 20% fetal bovine serum (FBS), 50 μM tert-butylhydroquinone (tBHQ), or 10 ng/mL tumor necrosis factor alpha (TNFa) were used to activate serum response element (SRE), antioxidant response element (ARE), or nuclear factor kappa B (NF-KB) responsive element. Bioluminescence signals were acquired 16 h post induction. An un-transfected sample was used for background subtraction and an un-induced sample was used as a negative control.

### Example 6: Design of Triple Luciferase System and the Directed Evolution of Luciferase to Improve Substrate Selectivity for Synthetic pyDTZ

To choose bioluminescent reporters that can generate different colors of emission, available luciferase-luciferin pairs that have been reported previously in literature were first screened. RLuc8 (nucleotide and polypeptide sequences provided herein as SEQ ID NOs. 13 and 14, respectively) is able to produce intense bioluminescence in a violet wavelength range (λmax: ˜405 nm) when methoxy-eCoelenterazine (me-eCTZ) was used as the substrate. Renilla luciferase (RLuc) is also known to be not tolerant to C-8 chemical modifications. It was reasoned that the blue-shifted emission might be due to dihedral angle twist caused by the C-6 methoxyl substitution. Therefore, a me-eCTZ analog, pyOMeCTZ, with a pyridyl substitution on C-2 was synthesized to improve the water solubility by taking advantage of the fact that pyridine-containing molecules can be readily turned into pyridinium salts. As a result, RLuc8-pyOMeCTZ pair is able to generate violet emission with λmax at about 416 nm, or yield a bioluminescence of about 380-470 nm.

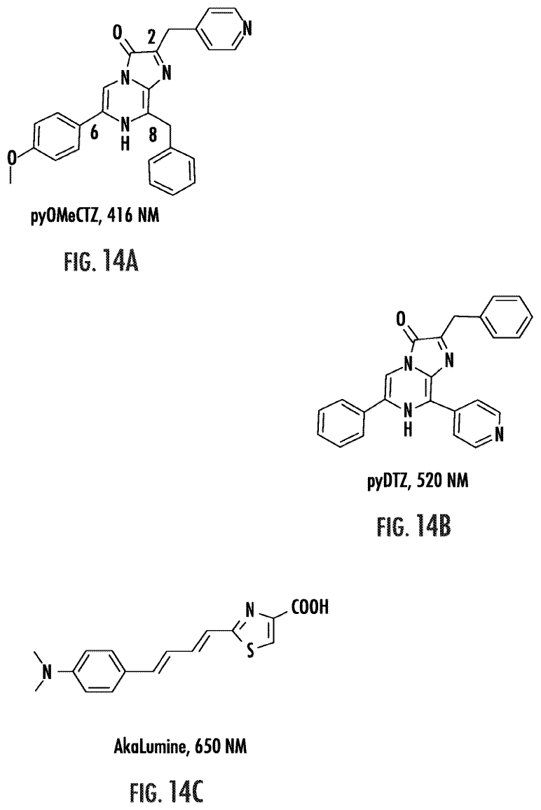

According to our result, teLuc is tolerant to a variety of C-8 chemical modifications, including both electronic and steric derivatives. Herein, pyDTZ that can emit green to yellow photons (λmax: about 530 nm, or yield a bioluminescence of about 480-600 nm) when paired with teLuc was synthesized. Since the emission wavelength between RLuc8-pyOMeCTZ and teLuc-pyDTZ pairs are well resolved, they are readily available to pair with Akaluc (nucleotide and polypeptide sequences provided herein as SEQ ID NOs. 15 and 16, respectively)-AkaLumine pair that produces near infrared (NIR) photons (λmax: about 650 nm, or yield a bioluminescence of about 600-750 nm; FIG. 14C) to yield a triple-color luciferase reporter system. It has been known that CTZ-utilizing murine luciferases do not have substrate cross-talk with D-luciferin-utilizing insect luciferases, so the remaining issue is the substrate selectivity between RLuc8 and teLuc to engineer a fully orthogonal triple-color luciferase system.

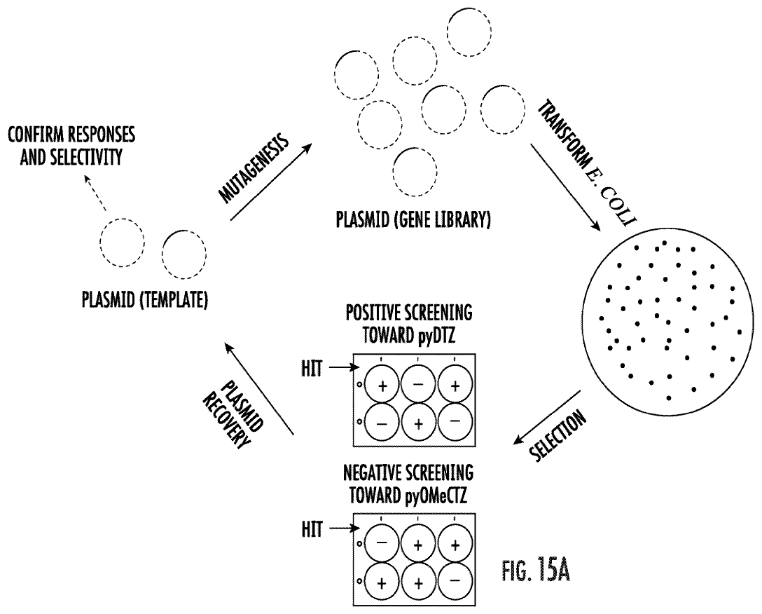

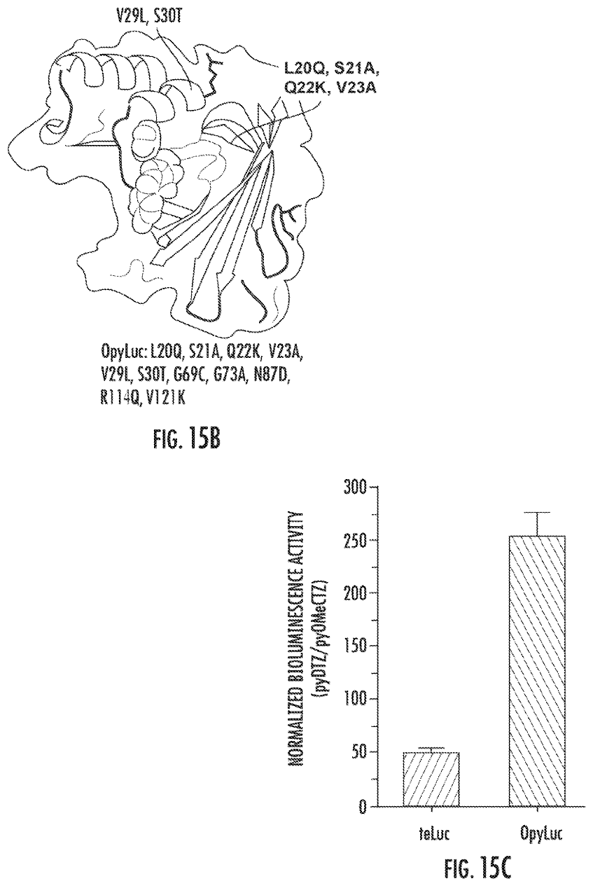

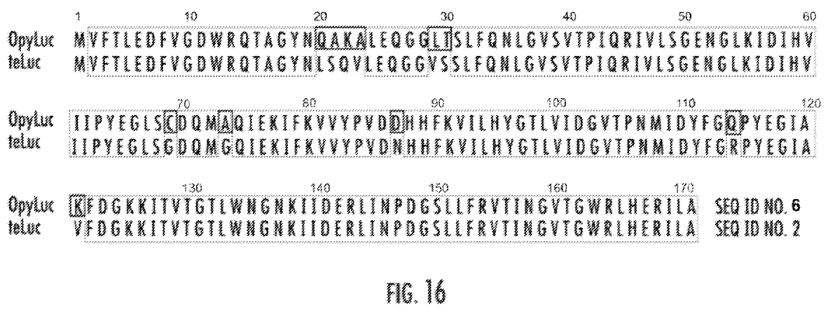

To address this issue, it was noticed that teLuc exhibited a substrate preference to pyDTZ over pyOMeCTZ by about 50-fold activity, suggesting that it might be feasible to engineer a mutant via directed evolution to more selectively access pyDTZ rather than pyOMeCTZ. Instead of screening the library for only enhanced bioluminescence output, a method was designed where the BL activity of the mutants to both of pyDTZ (positive screening) and pyOMeCTZ (negative screening) were screened in parallel. The “hit” mutants showing not only high specific activity in positive screening but also low activity in negative screening were selected for the next round selection (FIG. 15A). After 8 rounds of selection, a mutant (designated OpyLuc; SEQ ID NO. 6) carried 11 mutations was obtained (FIG. 15B, and FIG. 16) and showed about 250-fold selectivity to pyDTZ over pyOMeCTZ (FIG. 15C). Notably, Q22K⁺ and V23A mutations were acquired during random mutagenesis, suggesting residues nearby the catalytic site contribute to the substrate selectivity.

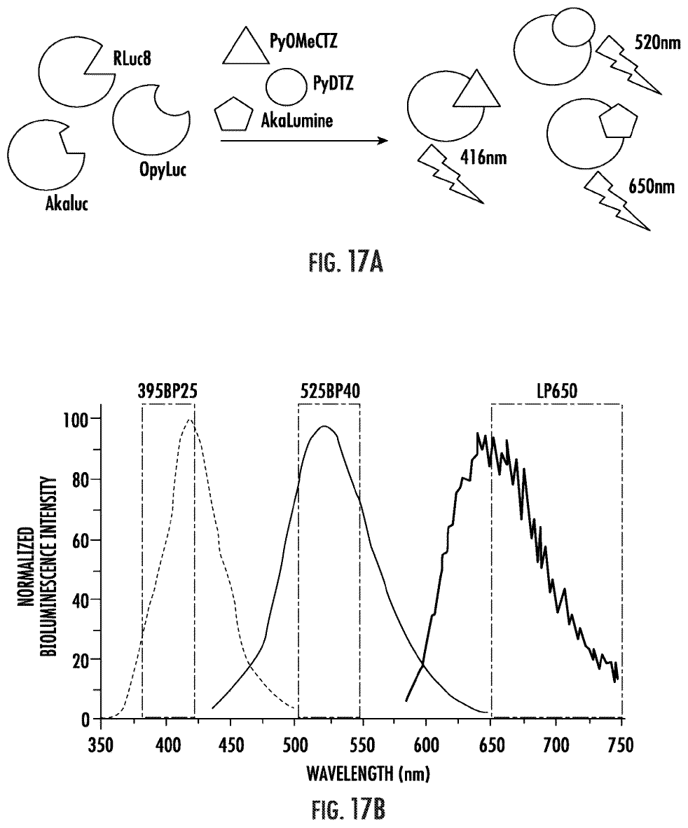

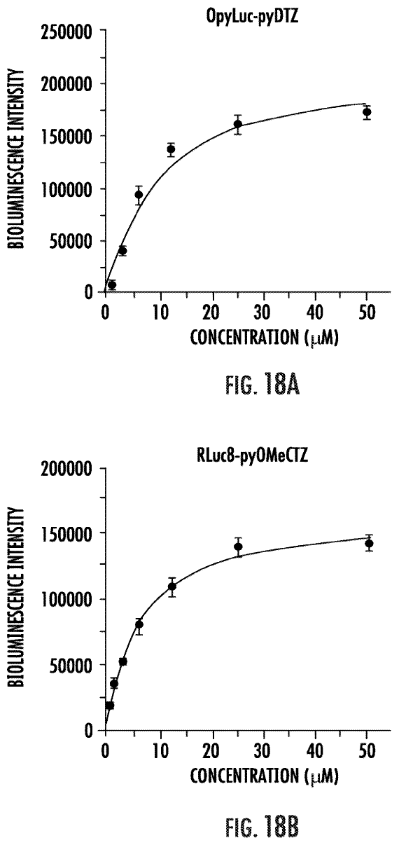

Collectively, provided herein are three luciferase-luciferin pairs (RLuc8-pyOMeCTZ; λmax: 416 nm, OpyLuc-pyDTZ; λmax: 520 nm, and Akaluc-AkaLumine; λmax: 650 nm) that can access its specific luciferin and produce distinct colors of emission across the visible spectrum (FIGS. 17A, 18A and 18B). Their emission spectra are well separated and with only minimal spectra cross-talk. These features allow researcher to either initiate a specific luciferase activity by adding its corresponding luciferin substrate or scan the full spectra or use commercial filters to determine individual luciferase signals, thereby providing flexible data acquisition methods for any chosen purpose (FIG. 17B).

### Example 7: Triple Luciferase System Produces Orthogonal BL Signals in Purified Enzyme and in Transfected HEK293T Cells

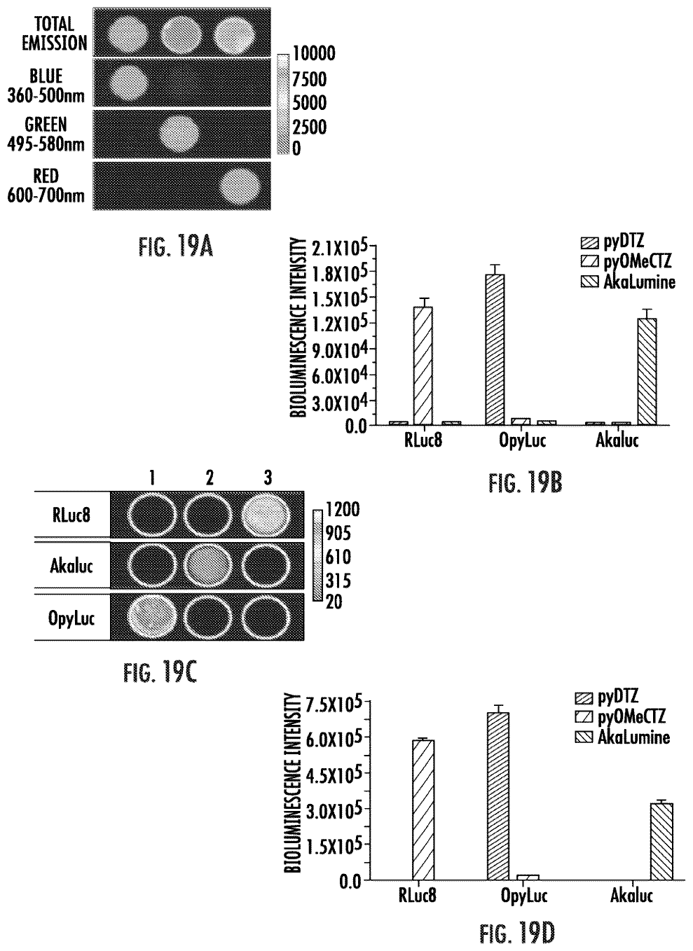

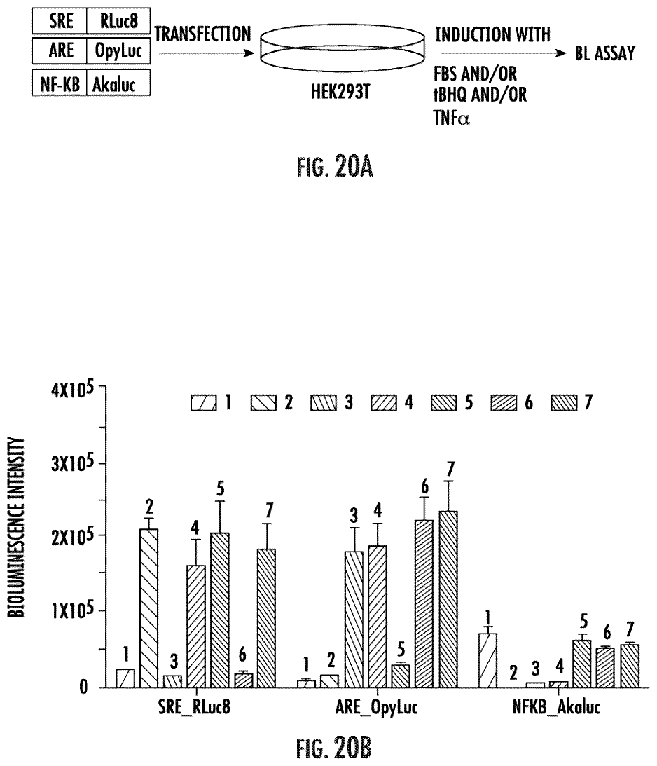

To validate that the emission of these three luciferase-luciferin pairs are indeed spectrally separated, and can be resolved by filters, recombinant luciferases were first purified from E. coli and the respective BL signals were imaged with/without 360-500 nm, 495-580 nm, or 600-700 nm bandpass filters (FIGS. 19A and 19B). The result indicated RLuc8-pyOMeCTZ, OpyLuc-pyDTZ, and Akaluc-AkaLumine pairs all give the highest signal under the filter set-up that matches its respective emission color, suggesting their emission spectra can be well separated simultaneously by a set of commercial filters. Since these signals can be recorded in the same time, it solved the need of sequential sampling by Promega Dual-Luciferase Reporter assay.

To demonstrate that the disclosed triple luciferase system is a practical tool to monitor gene expression levels in live mammalian cells, the photon flux of each luciferase-luciferin pair was first evaluated in the presence of a series of substrate concentrations. It would have been ideal to explore an optimal concentration for each luciferin (pyOMeCTZ, pyDTZ, and AkaLumine) that can provide a similar level of photon flux from individual luciferase-luciferin pair. Unfortunately, the photon flux of Akaluc-AkaLumine at saturated concentration is about 10-fold lower than that of RLuc8-pyOMeCTZ and OpyLuc-pyDTZ pairs, possibly due to its nature BL mechanism of ATP-dependency and two-step reaction. In order to at least keep the photon flux of RLuc8-pyOMeCTZ and OpyLuc-pyDTZ pairs at the same level, a condition containing 25 μM pyOMeCTZ, 10 μM pyDTZ, and 100 μM AkaLumine-HCl was selected as the “Optimal Mix” for live cell imaging. By comparing Optimal Mix and only its respective substrate, only OpyLuc exhibited slightly unspecific inhibition by Optimal Mix while RLuc8 and Akaluc remained unaffected.

Next, the performance of each luciferase was examined for in cellulo imaging in the presence of chosen luciferin concentration. The indicated luciferin substrate solution was injected into luciferase-expressing HEK 293T cells in a 96-well plate. As expected, pyDTZ initiated the BL emission only in the presence of OpyLuc. The excellent substrate selectivity was also observed in both Akaluc for AkaLumine and RLuc8 for pyOMeCTZ (FIGS. 19C and 19D). Taken together, the results indicated again that each of luciferase in the disclosed triple luciferase system can process its distinct substrate and generate distinguishable emission wavelength. To briefly sum up, each luciferase can be selectively activated by its specific luciferin and the emission photons can also be distinguished by wavelength, which provided the flexibility to monitor multiple transcriptional activities specifically, stepwise, or simultaneously as a versatile experimental design. Moreover, the equations to calculate the activities of the individual luciferases by splitting emissions is not necessarily required since the spectra cross-talks are minimized.

While reporter assays are typically performed in lysates from cultured cells, the disclosed triple luciferase system was also evaluated in lysates. The results indicated that RLuc8-pyOMeCTZ and OpyLuc-pyDTZ pairs showed about 2 to 3-fold higher BL signals in lysates while Akaluc-AkaLumine exhibited decreased signal even after supplementing with additional ATP. Therefore, using lysates is not required in the disclosed triple luciferase system because all three luciferins described here are cell-permeable and work well with intact mammalian cells. This feature is beneficial to expand the real-time measurement of BL assays without lysing cultured cells.

### Example 8: Monitor Serum Response, Antioxidant, and NF-KB Promoter Activities in HEK293T Cells by Triple Luciferase System

Subsequently, the disclosed triple luciferase system was used to monitor three signaling pathway activations in HEK293T cells where each of the luciferase expression was under control by a growth factor-regulated promoter element (serum response element, SRE), a Nrf2-antioxidant response element (ARE), or a transcription factor-nuclear factor kappa B (NF-KB) responsive promoter element (Table 5). A reporter system was designed based on SRE promoter driving the expression of RLuc8, ARE promoter driving the expression of OpyLuc, and NF-KB promoter driving the expression of Akaluc. The response element promoters can be specifically activated by its respective stimuli—fetal bovine serum (FBS), tert-butylhydroquinone (tBHQ), and tumor necrosis factor alpha (TNFa) (FIG. 20A).

TABLE 5
OLIGONUCLEOTIDES USED IN THIS STUDY

| Oligo Name | SEQ ID NO: | Nucleotide Sequence (5′→3′) |
|---|---|---|
| L20D21NNK-F | 29 | CAGACAGCCGGCTACAACNNKNNKCAAGTC |
| | | CTTGAACAGGGAGGTGTG |
| L20D21NNK-R | 30 | CACACCTCCCTGTTCAAG |
| | | GACTTGMNNMNNGTTGTAGCCGGCTGTCTG |
| 27VSSNNK-F | 31 | CAAGTCCTTGAACAGGGAGGTNNKNNKNNKTTG |
| | | TTTCAGAATCTCGGGGTG |
| 27VSSNNK-R | 32 | CACCCCGAGATTCTGAAACAAMNNMNNMNNAC |
| | | CTCCCTGTTCAAGGACTTG |
| pBAD-F | 33 | ATGCCATAGCATTTTTATCC |
| pBAD-R | 34 | GATTTAATCTGTATCAGG |
| HindIII-pyr-F-Koz | 35 | AATAAAGCTTGCCGCCACCATGGTCTTCACTCTC |
| pyr-R-XhoI | 36 | GGGATTTTAATTCTCGAGTACGCAGAAATGCGTTCAT |
| | | GCA |
| Aka-F-HindIII- | 37 | ATTATAAAGCTTGCCGCCACCATGGAAGATG |
| Kozak | | |
| Aka-R-XhoI | 38 | CCAAAACATTAAGATTACTCTCGAGTACACACGGCG |
| | | ATCTTGCCCTC |
| Aka-F-XhoI | 39 | CTTATACTCACACATGGAAGAGATGCAAAAACATTA |
| Aka-R-HindIII | 40 | TTGCCAAGCTTACACGGCGATCTTGCCGTCCTTCTT |
| HindIII-mScarlet-F- | 41 | ATTATAAAGCTTGCCGCCACCATGGTGAGCAAGGGC |
| Koz | | GAGGCAGT |
| mScarlet-F-XhoI | 42 | ATAACTCGAGCATGGTGAGCAAGGGCGAGGCAGTG |
| pyr-R-HindIII | 43 | TTGCCAAGCTTACGCCAGAATGCGTTCATGCA |
| mScar-NNK-pyr-F | 44 | GAGGGCCGCCACTCCACCGGANNKACTCTCGGG |
| mScar-NNK-pyr-R | 45 | GATTTTCTTGGCCCA |
| | | CACAAATCCCGGGAGAGTMNTCCGGTGGTGGCGGCC |
| | | CTC |

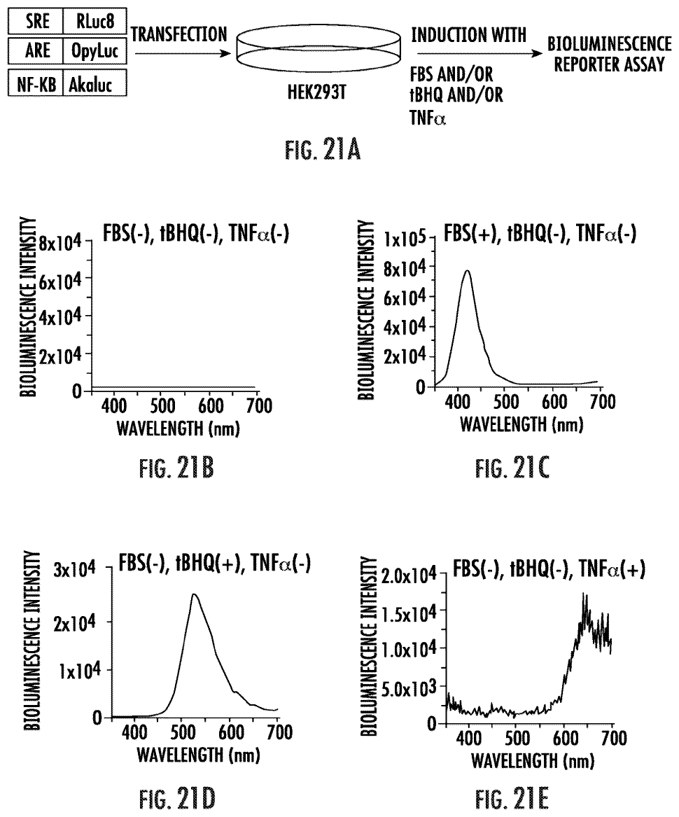

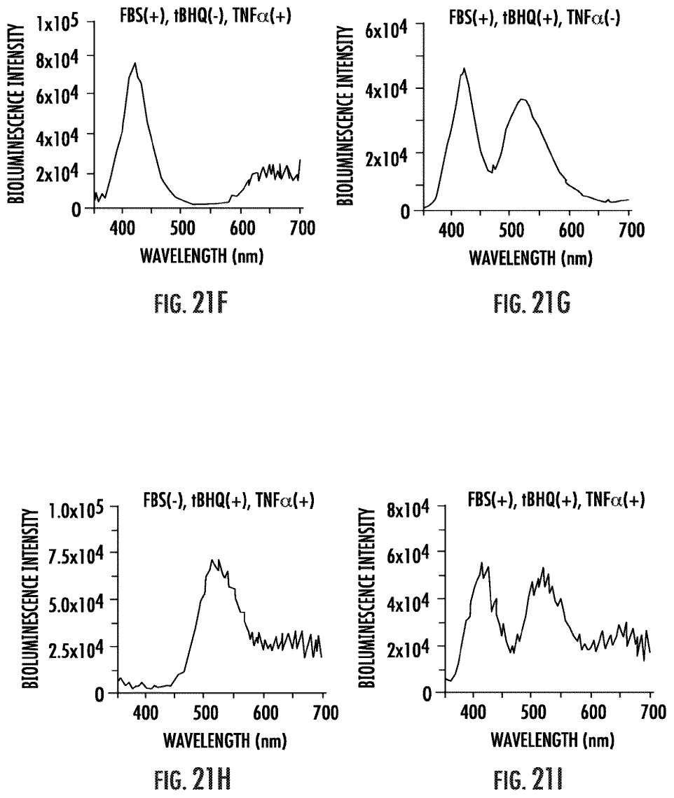

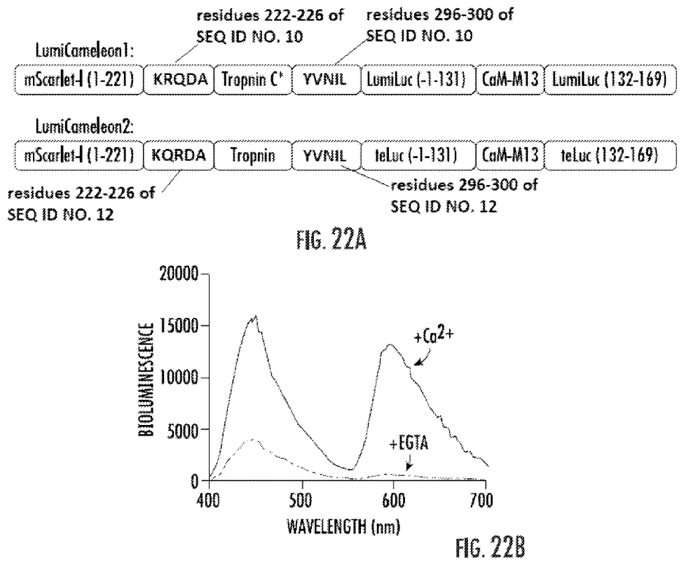

The basal promoter activities of all SRE, ARE, and NF-KB response elements were low (FIG. 6A). The individual luciferase activity was measured (RLuc8, OpyLuc, and Akaluc) by adding its corresponding luciferin (pyOMeCTZ, pyDTZ, and AkaLumine). As expected, treating with single stimuli enabled the activation of its specific pathway and drove the downstream expression of genetically encoded luciferase. Stimulating the cells with either two or three stimuli resulted in activation of multi-pathway and were reported correctly by our triple luciferase system (FIG. 20B). Next, the emission spectra were recorded after injection of Optimal Mix solution to the transfected HEK293T cells (FIG. 20B and FIGS. 21C, D, and E). Due to the fact that all three emission spectra are well-separated, this system demonstrated a proof-of-concept of simultaneous recording of three pathway activations. Again, the results suggested that there is no cross-reactivity between each luciferase-luciferin pair, providing an advantage in future studies that require either sequential or simultaneous multicomponent monitoring.

Herein, the ability of the disclosed triple luciferase system to monitor the simultaneous activation of two or all three labeled pathways was demonstrated. The ability to qualitatively detect three signaling activation states after treating with stimuli mixtures was demonstrated (FIGS. 20B and 21F, G, H, and I).

Next, the promoters were switched over to drive the downstream luciferase expression by using an alternative combination (NF-KB-RLuc8, SRE-OpyLuc, and ARE-Akaluc). After inducing with all stimuli (TNFa, FBS, and tBHQ), the signals from RLuc8 and OpyLuc were obvious as expected, while the signal from Akaluc was overwritten by the broad emission tailing of OpyLuc (FIG. 21). This result suggested that Akaluc is only suitable to monitor strong promoter activity when paired with the other two luciferases, because the photon flux of Akaluc is relatively lower. This is a factor that needs to be taken into account when researchers conducting the initial experimental design.

These data support the use of the new luciferase-luciferin pairs for assays of cell signaling pathways. Particularly, the data summarized in FIG. 21 suggest that the use of the disclosed luciferase-luciferin pairs can allow for the monitoring of particular cell signaling pathways, which can be implemented for candidate compound and drug screening applications.

### Example 9: Akaluc-Akalumine Pair is More Suitable as an Internal Control

As mentioned above, it is recommended to normalize the BL assay results by an internal control for cell number, and transfection efficiency normalizations. Another set of plasmids containing NF-KB-RLuc8, SRE-OpyLuc, and a control of the constitutively active cytomegalovirus (CMV) promoter (CMV-Akaluc) was prepared. The cells were transfected with all three plasmids and incubated with two stimuli (TNFa and FBS) for 16 h. In this case, the BL signal was selectively triggered from individual luciferase by adding its respective luciferin to intact cells (FIG. 6A). The BL kinetics were monitored for each luciferase after the addition of its respective luciferin (FIG. 6B). All three luciferase generated BL signal, and the BL signal attenuated by the function of time. The advantage of incorporating an internal control here is not only limited to the normalization of cell number and transfection efficiency of each sample, but also the kinetic of each BL signal can be normalized to give a more constant ratio readout, which is more amenable to high-throughput screening (FIG. 6C). Thus, this system enabled the monitoring of two signaling activations and improved the accuracy of assay by including another orthogonal control to exclude factors such as cell number, transfection efficiency, and BL emission kinetic.

### Example 10: Analysis of Bioluminescent Ca²⁺ Biosensors

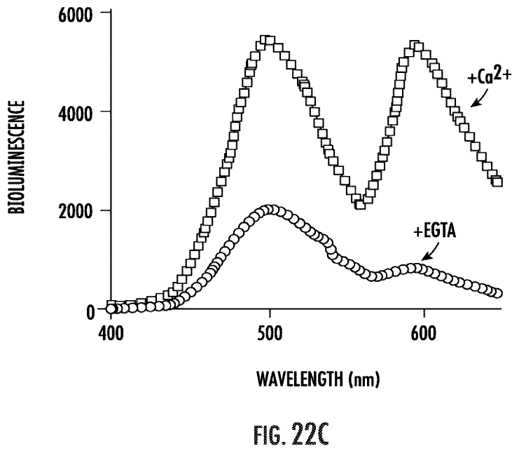

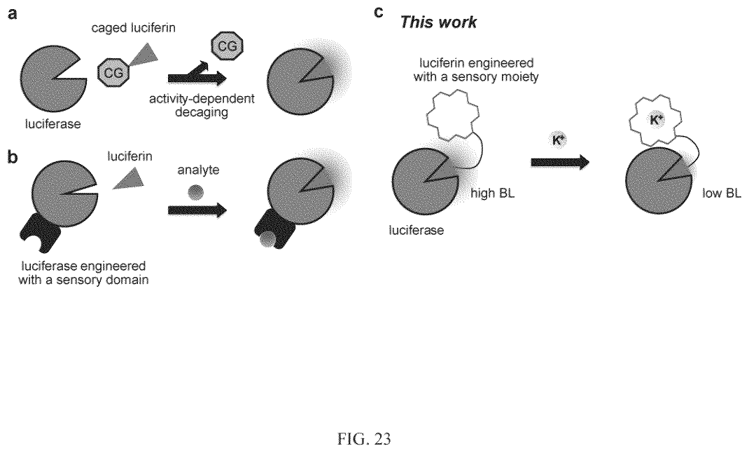

Ca²⁺ is one of the most important signaling cations in biological systems. Bioluminescent Ca²⁺ biosensors are expected to have broad applications in non-invasive imaging and drug screening. Recent studies have reported a few bioluminescent Ca²⁺ biosensors based on NanoLuc. To address the limitations of these existing sensors, such as small dynamic range, low brightness, and/or relatively blue emission, Ca²⁺ biosensors based on the disclosed brighter and redder luciferase-luciferin pairs were developed. Moreover, in contrast to existing approaches, multiple Ca²⁺ binding elements were introduced to modulate bioluminescence through two different mechanisms. In particular, a calcium sensory element (e.g., a modified Troponin C) was sandwiched between a LumiLuc and Scarlet-I BRET pair. After testing several linker lengths, a prototypic biosensor was derived, whose bioluminescence at wavelengths longer than 600 nm increases by about 4-fold in response to Ca²⁺. Further, calmodulin and M13 were inserted between the residue 133 and 134 of Lumiluc to modulate its intensity, resulting in LumiCameleon1 (FIGS. 22A and 22B; SEQ ID NOs. 9 and 10) showing a total of greater than about 20-fold increase of greater than about 600 nm emission in response to Ca²⁺. The brightness of LumiCameleon1 in the Ca²⁺: saturated state is about 52% of LumiScarlet. This sensor is thus well suited for in vivo and in vitro applications, because of its high brightness, large dynamic range, and red-shifted emission compared to any previously reported NanoLuc-based Ca²⁺ sensor. Along the same line, a similar biosensor based on teLuc was created. The resultant LumiCameleon2 (FIGS. 22A and 22C; SEQ ID NOs. 11 and 12) also showed a large bioluminescence increase in response to Ca2+.

### Discussion of Examples 6-10

Herein, substrate selectivity was utilized to engineer a mutually orthogonal luciferase-luciferin pair for multiplexed cell-based BL assay. In combination with RLuc8 and Akaluc, this triple-color BL system features the selectivity of synthetic substrates and production of well separated emission spectra from 400 nm to 650 nm. Several advantages of previous bioluminescence technology (Table 6) were combined to develop a spectral-resolved triple-color BL system, which provides flexible and convenient approaches to monitor multiple biological events in either qualitative or quantitative manners.

New bioluminescent Ca²⁺ biosensors were also developed based on the modified luciferase compounds disclosed herein. These bioluminescent Ca²⁺ biosensors showed large bioluminescence increase in response to Ca²⁺, making them well suited for in vivo and in vitro applications.

TABLE 6
QUALITATIVE COMPARISON OF THIS STUDY AND
COMMERCIAL LUCIFERASE REPORTER SYSTEMS

| | | | Pierce | Orthogonal |
|---|---|---|---|---|
| | Promega | | Cypridina- | Triple |
| | Dual- | Promega | Firefly | Luciferase |
| Feature/Assay | Luciferase | Chroma- | Luciferase | Assay |
| System | Assay | Glo | Dual Assay | (this work) |
| Number of | 2 | 1 | 2 | 3 |
| Enzymes | | | | |
| Types of | Rluc, | 2 | VLuc, | 3 |
| Enzymes | FLuc | CBLucs | Red FLuc | |
| Gene | low | >99% | low | low |
| Identity | | | | |
| Orthogonal | Yes | No | Yes | Yes |
| Signals | | | | |
| Simultaneous | No | Yes | Yes | Yes |
| Detection of | | | | |
| 2 Signals | | | | |
| Detection of | No | No | No | Yes |
| 3 Signals | | | | |
| Data | No | Yes | No | Yes/No |
| Calculations | | | | |
| Required | | | | |
| Luminometer | No | Yes | Yes | Yes/No |
| Filters | | | | |
| Required | | | | |
| Single | No (CTZ, | Yes | Yes | Yes (pyDTZ, |
| Reagent | D-luciferin) | (D-luciferin) | (Vargulin, | pyOMeCTZ, |
| Solution | | | D-luciferin) | Akalumine- |
| | | | | HCl) |
| Emission | Yes | No | Yes | Yes |
| Signals Well- | | | | |
| Separated | | | | |
| Cell Lysis | Yes | Yes | Yes | Yes/No |
| Required | | | | |

By using this triple luciferase system, it was demonstrated that the activations of cell signaling can be detected simultaneously or separately from live cells in a single experiment where each individual BL signal can be distinguished from the other two luciferase-luciferin pairs. It is expected that there is also an ability to combine newly discovered luciferase-luciferin pairs to independently activate even more innate processes in the same sample to study the cross-talks of cellular signaling pathways. Moreover, multiplexed BL assay is compatible with modern genetically encoded fluorescent biosensors to further investigate complex biological events via functional imaging. The development of such a versatile tool ensures an accurate and precise analysis of signaling pathways which will extend to study other physiologically transcriptional activation and is critical to improve the design and screening of new drugs, as well as the diagnosis and treatment of disease.

### Development of Potassiorin Luciferin and BRIPO

### Selected Abbreviations

BRIPO bioluminescent red indicator for potassium;
BL bioluminescence;
BLI bioluminescence imaging;
FLuc Firefly luciferase;
FP fluorescent proteins;
RET resonance energy transfer;
K⁺ potassium ions;
Na⁺ sodium ions;
RFP red fluorescent protein;
K_{M} Michaelis constant;
K_{d} apparent dissociation constant;
AAVs adeno-associated viruses.

This disclosure additionally introduces novel bioluminescence imaging methods; and, in particular, a method for studying K⁺ dynamics. Developed embodiments include potassiorin, a luciferin responsive to K⁺. Additional embodiments include the engineered artificial luciferase enzyme BRIPO developed to work in synergy with potassiorin. As below discussed, the BRIPO-potassiorin system demonstrated robust K⁺-dependent bioluminescence quenching in purified proteins, cell lines, primary neurons, and live mice, validating the effectiveness of this new system.

Interestingly, the presence of K⁺ was observed to enhance the affinity of potassium only minimally for the enzyme and slightly reduce the bioluminescence quantum yield, but it resulted in a notable decrease in photon production rate. This observation suggests that K⁺ binding to potassiorin allosterically modulates the enzyme activity, potentially through interactions involving the mutated residues. In the protein engineering process, the introduction of S260R and V261W mutations was identified as particularly preferable for augmenting the response magnitude and selectivity of the BRIPO-potassiorin system. It is envisioned that, in principal, the positively charged guanidinium group in R260 could potentially interact with the crown ether ring of potassiorin, while W261 may engage in a cation-π interaction when K⁺ binds to the crown ether ring.

Importantly, the further disclosed and described K-induced turn-off response provides a practical advantage: the system exhibits a bioluminescence turn-on response when cells are activated physiologically; since, under these conditions, K⁺ efflux occurs through K⁺ channels, leading to hyperpolarization of the cell membrane. In addition, embodiments of the disclosed method advantageously overcome the limitations of traditional K-detection techniques like ion-selective electrodes, flame photometry, and fluorescent indicators, which face challenges in tissue- and organism-level studies. By enabling visualization and study of K⁺ dynamics from live cells to animals, the embodiments disclosed and described new avenues for understanding the role of K in physiology and disease.

Additionally, as aforementioned, this disclosure introduces a novel approach for generating bioluminescent indicators by modifying the luciferin molecule with an analyte-binding moiety. In embodiments, it was surprisingly found that extending the luciferin molecule through the C2 aromatic ring of imidazopyrazinone is a viable option, as NanoLuc-derived luciferases display tolerance to such structural modifications while remaining sensitive to the modulation caused by analyte binding. The effectiveness of this strategy was successfully demonstrated through the development of the herein disclosed indicator for K⁺. Further embodiments and applications of the techniques taught herein also led to the development of a prototype bioluminescent Na⁺ indicator by substituting the 18-member crown ether moiety in potassiorin with a 15-member ring. It is further envisioned that the sensory luciferin may be enhanced by incorporating ligands with higher affinity to K⁺ and Na⁺, aiming to improve sensitivity and enable the detection of extracellular K⁺ as well as intracellular and extracellular Na⁺.

Prior research has utilized the caged luciferin strategy (FIG. 23, a) to successfully create indicators for metal ions such as Cu²⁺ and Fe²⁺, contingent upon the catalytic capability of these transition metals in catalyzing decaging reactions. In these cases, the bioluminescence intensity is influenced by the kinetics of decaging and luciferin clearance, which may not accurately reflect the spatiotemporal dynamics of the metals. In contrast, the approaches disclosed herein utilize binding and dissociation kinetics; which often occur more rapidly than decaging reactions and luciferin clearance. Moreover, the approaches disclosed herein can be extended to metal ions that are not amenable to decaging reactions, as well as broader targets including anions and nonionic entities. Through the integration of various sensory moieties into the luciferin structure, embodiments of the disclosed methodology has the potential to significantly broaden the spectrum of bioluminescent indicators.

Overall, this disclosure provides advancements in bioluminescent sensors, allowing for the creation of versatile indicators that can be adapted to monitor various ions, molecules, and molecular interactions. The flexibility in sensor design opens new avenues for broad applications of BLI, which will enhance the understanding of biological systems and drive forward biomedical research.

### Results

### Design and Synthesis of Potassiorin

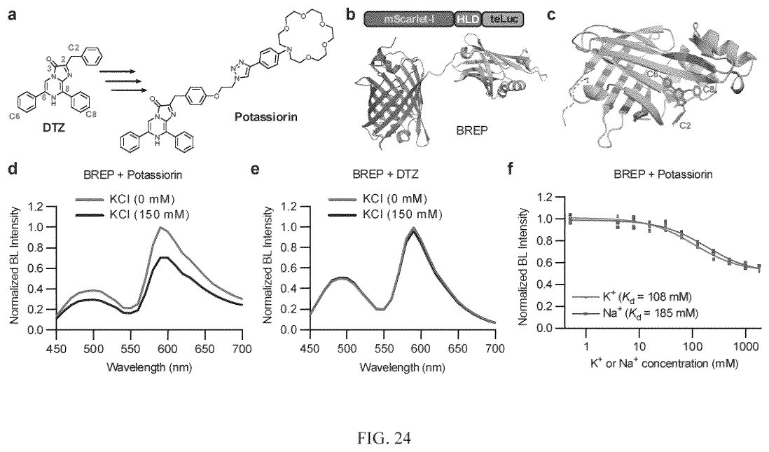

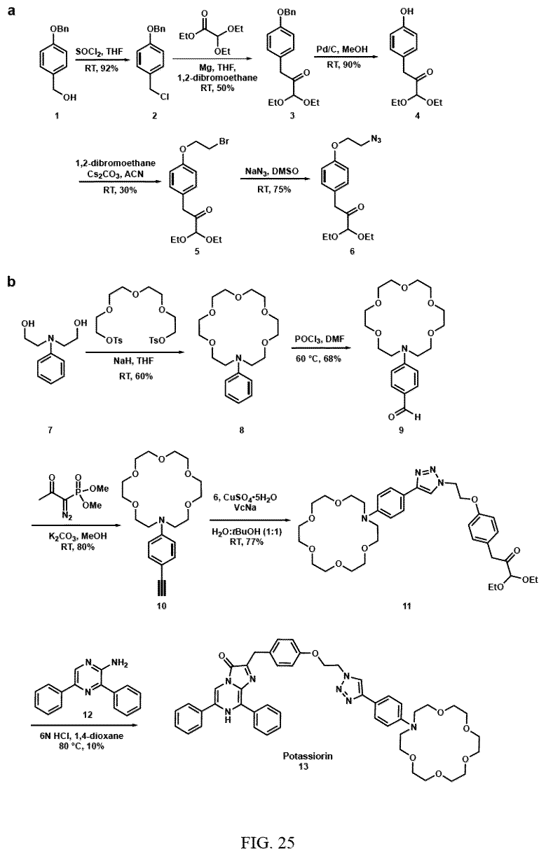

A prior disclosure details a NanoLuc variant called teLuc, which emits teal bioluminescence at around 500 nm when combined with the synthetic luciferin DTZ (FIG. 24, a). Notably, DTZ could be readily synthesized from commercially available reagents in just two steps with a good yield. The red-shifted emission of teLuc compared to NanoLuc, led to the development of BREP (FIG. 24, b), a fusion construct between teLuc and a red FP (RFP) mScarlet-I, emitting approximately 60% of its total emission above 600 nm. BREP enables deep-tissue photon penetration and has emerged as one of the most powerful luciferases for in vivo BLI. Building upon these findings, we selected DTZ and BREP as the foundations for the current disclosure.

K⁺ is the most abundant intracellular cation, with a high concentration of 140-150 mM within cells. It plays a critical role in generating functional activity in muscle cells, neurons, and cardiac tissue. To address the limited methods available for tracking K⁺ in living systems, we aimed to develop a novel bioluminescent K⁺ indicator by incorporating a K-binding moiety into DTZ. Specifically, we selected a crown ether called 1-aza-18-crown-6, known for its ability to form a complex with K⁺, to derivatize DTZ.

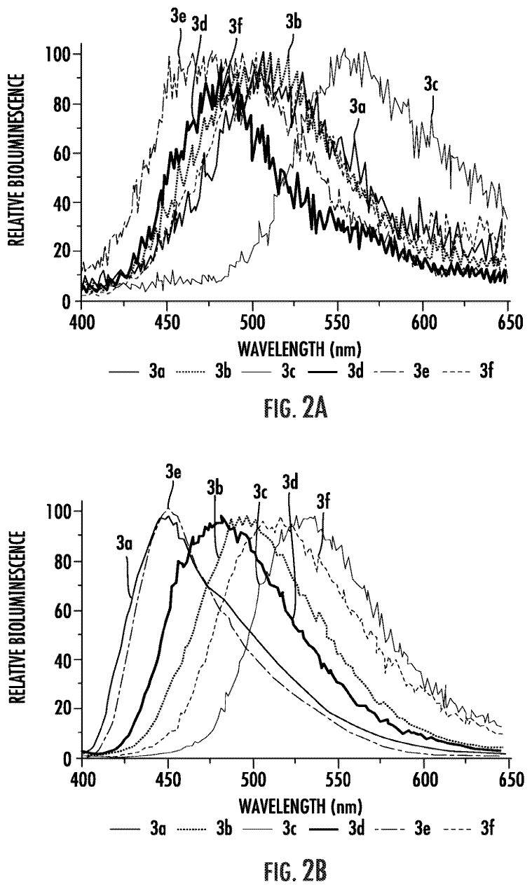

To overcome the challenge of not having a co-crystal structure of NanoLuc and its substrate, we utilized a previously generated docking structure of CTZ in NanoLuc (FIG. 24, c). From this model, we deduced that installing the 1-aza-18-crown-6 moiety through C6 or C8 of the imidazopyrazinone core of the substrate would likely result in the complete loss of bioluminescence activity due to their buried positions and the inability of the putative substrate-binding pocket to accommodate the size of the K⁺-binding moiety. However, we identified the aromatic ring at the C2 position of imidazopyrazinone as a promising site for installing the K⁺-binding moiety, as it extends outside of the putative substrate binding pocket of the luciferase enzyme (FIG. 2, c). Notably, this rationale, which was derived from the docking model, is consistent with a recently available co-crystal structure of NanoLuc and its inactive substrate analog.

We designed a DTZ analog called potassiorin, which incorporates the 1-aza-18-crown-6 moiety extended through the C2 position (FIG. 24, a). The synthesis of potassiorin involved multiple steps. Briefly, starting from commercially available 4-benzyloxybenzyl alcohol (compound 1 in FIG. 25), we synthesized 3-(4-(2-azidoethoxy)phenyl)-1,1-diethoxypropan-2-one (6) in five steps with an overall yield of 9.3% (FIG. 25, a). Simultaneously, we prepared N-(4-ethynylphenyl) aza-18-crown-6 (10) in three steps from N-phenyldiethanolamine (7), with an overall yield of 32.6%. The subsequent Cu (I)-catalyzed azide-alkyne cycloaddition (CuAAC) reaction between compounds 6 and 10 produced 11 in a 77% yield (FIG. 25, b). Finally, we employed our previously established procedure to synthesize 5-diphenylpyrazin-2-amine (12), which was then subjected to an acid-catalyzed cyclization reaction with 11, resulting in the final product, potassiorin, with a 10% yield (FIG. 25, b).

Initial Characterization of Potassiorin with BREP

After synthesizing potassiorin, we assessed the compound using purified BREP protein. We measured the emission spectra of BREP and potassiorin in the absence and presence of 150 mM K⁺ ions (FIG. 2, d). The results indicated that the presence of 150 mM K⁺ led to a reduction in bioluminescence by approximately 30%. In contrast, the bioluminescence of BREP and DTZ (as a negative control) showed only marginal changes in response to K⁺ (FIG. 2, 3). Furthermore, we examined the bioluminescence of BREP and potassiorin in the presence of various concentrations of K⁺ or Na⁺. Through our analysis, we determined the apparent affinities for K⁺ and Na⁺ (i.e., the concentrations to cause 50% of the overall bioluminescence change) to be 108 mM and 185 mM, respectively (FIG. 2, f). These findings suggest that potassiorin exhibits

K-dependent bioluminescence, although further improvements are necessary to enhance the dynamic range and selectivity towards K⁺ over Na⁺.

Engineering BREP into BRIPO for Enhanced Responsiveness

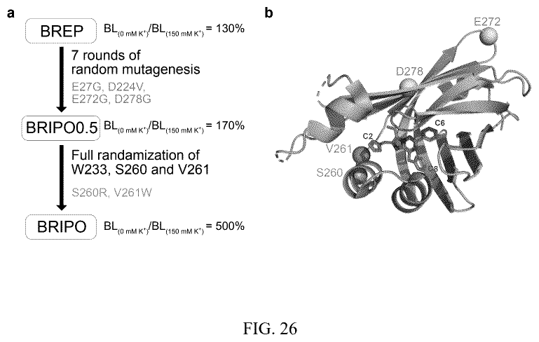

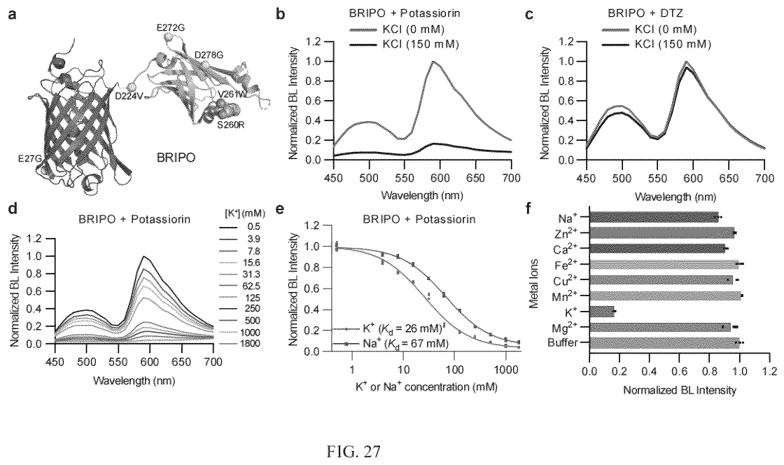

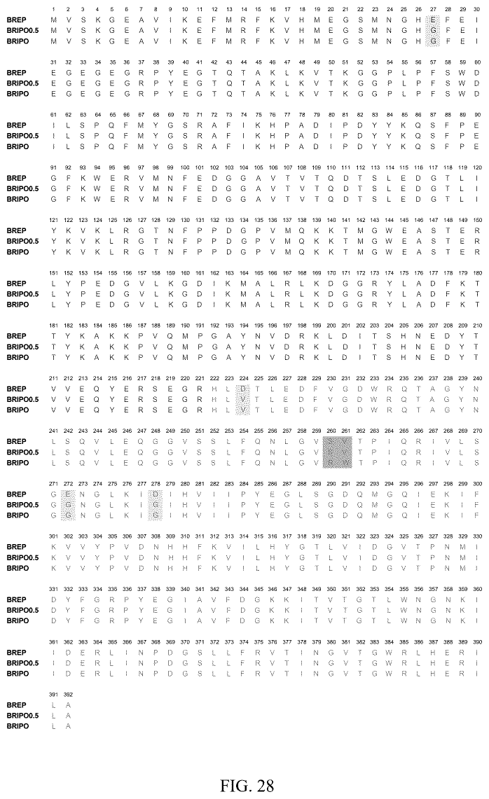

To enhance the dynamic range and selectivity, we employed random mutagenesis on BREP using error-prone PCRs. We screened the resulting gene library and selected clones exhibiting large K-dependent bioluminescence changes. These clones were subjected to counter-selection to ensure minimal bioluminescence changes in response to Na⁺. We conducted seven rounds of random mutagenesis and screening but observed only marginal improvement (FIG. 26). To further enhance performance, we turned to multi-site-directed mutagenesis. By utilizing the docking model, we identified and simultaneously mutated three amino acids (W233, S260, and V261) near the C2 position of the luciferin. Through screening this library, we successfully identified a mutant that exhibited a remarkably improved response magnitude and selectivity. This mutant, named BRIPO, contains a total of six mutations from the original BREP sequence (FIG. 27, a and FIG. 28). Notably, the S260R and V261W mutations obtained during the final step of engineering played a crucial role in enhancing the performance (FIG. 26, a). The co-crystal structure of NanoLuc and its substrate analog reaffirmed that these mutations are close to the C2 position of the luciferin substrate (FIG. 26, b), suggesting their ability to influence the interactions between potassiorin and the enzyme.

In the presence of BRIPO and potassiorin, 150 mM K⁺ resulted in a remarkably six-fold decrease in bioluminescence intensity (FIG. 5, b). Conversely, when BRIPO was combined with DTZ, the responsiveness to K⁺ was minimal (FIG. 27, c). Further titration experiments involving varying concentrations of K⁺ and Na⁺ revealed that the apparent affinities of the BRIPO and potassiorin pair for K⁺ and Na⁺ have been altered to 26 mM and 67 mM, respectively (FIGS. 27, d and e). Since the intracellular concentration of Na⁺ is approximately ten times lower than that of K⁺, the BRIPO-potassiorin system should exhibit sufficient specificity to accurately sense intracellular K⁺ levels over Na⁺.

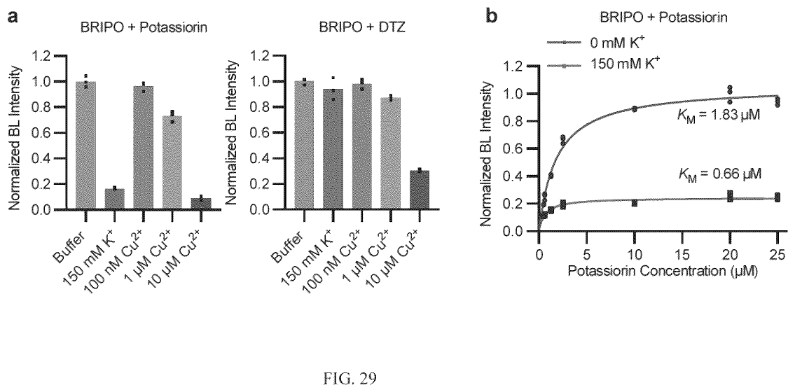

To validate the specificity of the BRIPO-potassiorin system, we conducted additional tests using a range of cations at physiologically relevant or higher concentrations (FIG. 27, f). The results confirmed the system's selectivity towards K⁺. Notably, high concentrations of Cu²⁺ led to bioluminescence quenching (FIG. 29, a), which is consistent with a previously documented mechanism involving the Cu²⁺-mediated oxidation of the luciferin. However, these high Cu²⁺ concentrations are unlikely to be physiologically relevant.

Moreover, we conducted additional investigations into the potassiorin concentration dependency of BRIPO bioluminescence in the presence or absence of 150 mM K⁺ (FIG. 29, b). Our findings indicate that the presence of K⁺ enhances the binding of potassiorin to the enzyme, resulting in a lower Michaelis constant (K_{M}). However, this slightly enhanced affinity does not lead to increased bioluminescence. Instead, we observed a nearly five-fold reduction in maximal photon production rates (Vₘₐₓ) and a small decrease in quantum yields (Table 1). These results support that a K⁺-dependent reduction in enzyme catalysis is the primary factor contributing to the observed bioluminescence turn-off response.

TABLE 1
Apparent Michaelis constants (K_{M}), relative maximal photon
production rates (Vₘₐₓ) and quantum yield (QY) of the BRIPO-
potassiorin pair in the presence or absence of 150 mM K⁺.

| BRIPO- | | | |
|---|---|---|---|
| potassiorin | K_{M} (μM) | Relative Vₘₐₓ | QY (%) |
| + KCl | 0.66 | 1 | 1.75 |
| − KCl | 1.83 | 4.33 | 2.01 |

### Imaging Intracellular K⁺ Dynamics in Cultured Cell Lines

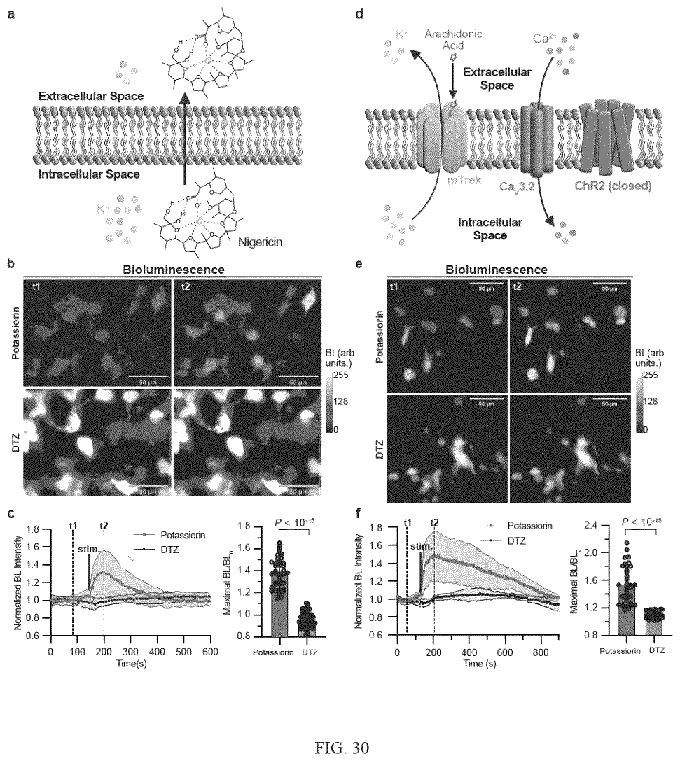

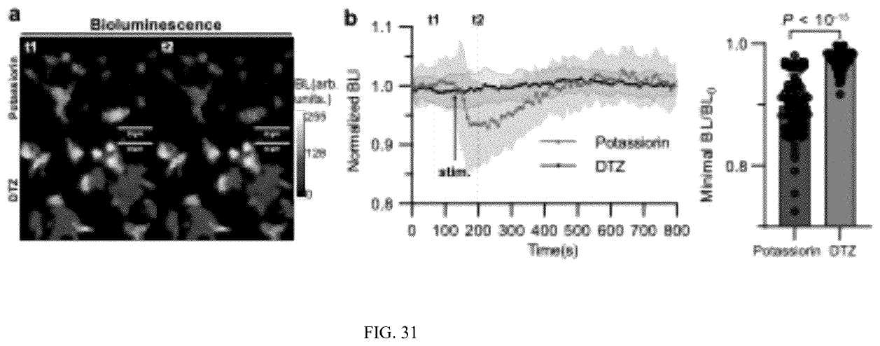

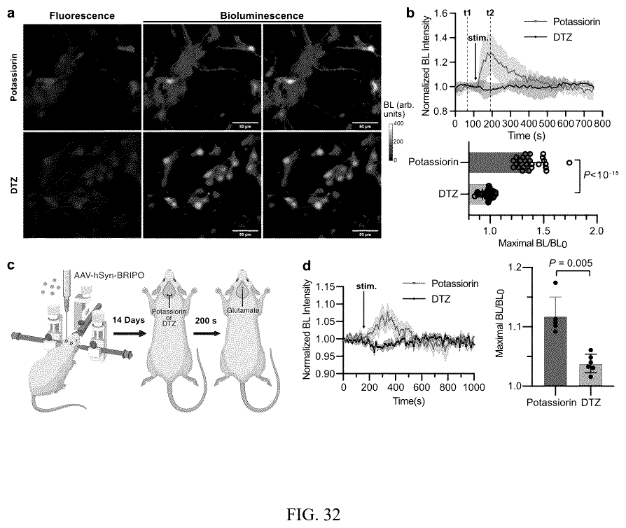

To investigate the ability of BRIPO and potassiorin to visualize K⁺ dynamics in live mammalian cells, we expressed BRIPO in HEK 293T cells and imaged the cells in a low K⁺ buffer supplemented with potassiorin. Inducing K⁺ efflux with a combination of nigericin (a K⁺ ionophore), bumetanide (an inhibitor of Na⁺/K⁺/2Cl⁻ cotransporter), and ouabain (an inhibitor of Na⁺, K⁺-ATPase pump) resulted in an approximately 30% increase in bioluminescence (FIG. 30 a-c). Furthermore, we replicated these experiments using HEK 293T cells in a high K⁺ (200 mM) buffer, facilitating the movement of K⁺ from the extracellular space to the intracellular space, resulting in an approximately 8% decrease in bioluminescence (FIG. 31). In addition, we expressed BRIPO in a HEK 293T cell line stably expressing a mouse leak K⁺ channel (mTrek) and a few other ion channels. We next used arachidonic acid to stimulate the mTrek channel, allowing K⁺ efflux to the extracellular low K⁺ space. This led to a nearly 50% increase in bioluminescence (FIGS. 30, d-f). For all three cases, control experiments with DTZ did not show much change in bioluminescence (FIG. 30 and FIG. 31). These findings support the effectiveness of the BRIPO-potassiorin system for selectively monitoring K⁺ dynamics in live mammalian cells.

### Imaging K⁺ Dynamics in Primary Neurons and Live Mice

Using the BRIPO-potassiorin system, we imaged K⁺ dynamics in primary mouse neurons. We transduced the neurons with adeno-associated viruses (AAVs) and imaged them in a low K⁺ buffer with potassiorin. Glutamate was used to activate the cells, causing membrane depolarization followed by repolarization due to K⁺ channel activation and K⁺ efflux. Around 40% of the examined neurons, which are likely to express glutamate receptors, exhibited robust bioluminescence increases (FIG. 32, a, b). In contrast, almost no cells in the control experiments using DTZ showed obvious changes in bioluminescence.

To further validate the effectiveness of the BRIPO-potassiorin system for in vivo imaging, we performed BLI in live mouse brains. We administered AAVs carrying the BRIPO gene into the hippocampal and cortical regions of mice. After three weeks of gene expression, we injected potassiorin and conducted time-lapse imaging on anesthetized mice placed in a dark box. Further delivery of glutamate resulted in notable increases in bioluminescence in all five mice injected with potassiorin, indicating the detection of K⁺ dynamics (FIG. 32, c, d). In contrast, control experiments using DTZ showed minimal changes in bioluminescence, which can be attributed to animal movement or alterations in blood flow. These findings provide strong evidence for the efficacy of the BRIPO-potassiorin system for in vivo imaging applications.

### Expanding the Approach for Na⁺ Sensing

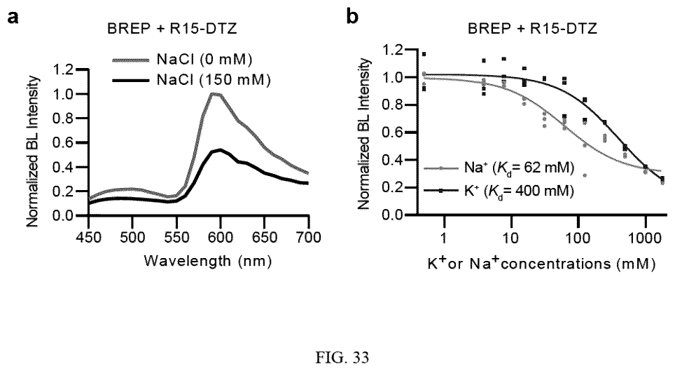

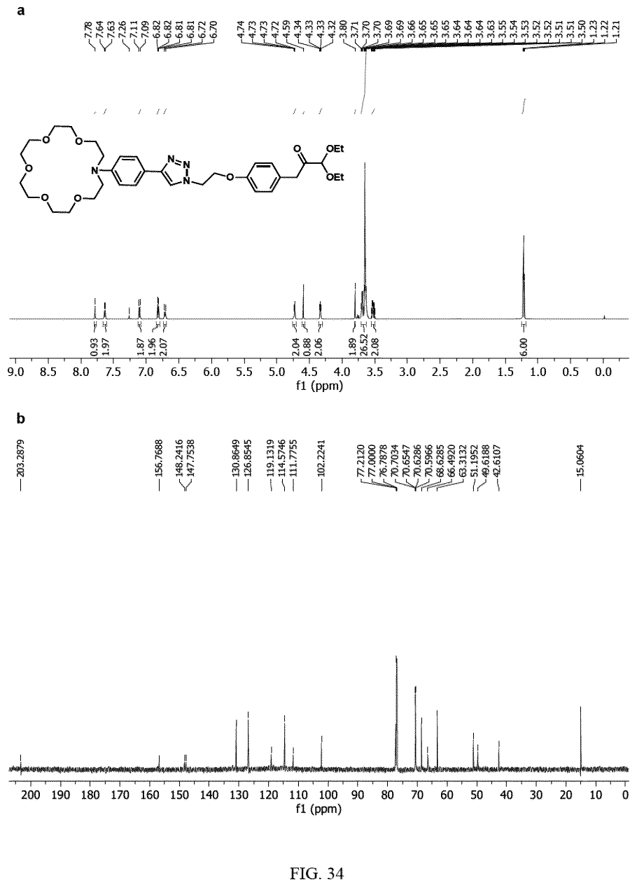

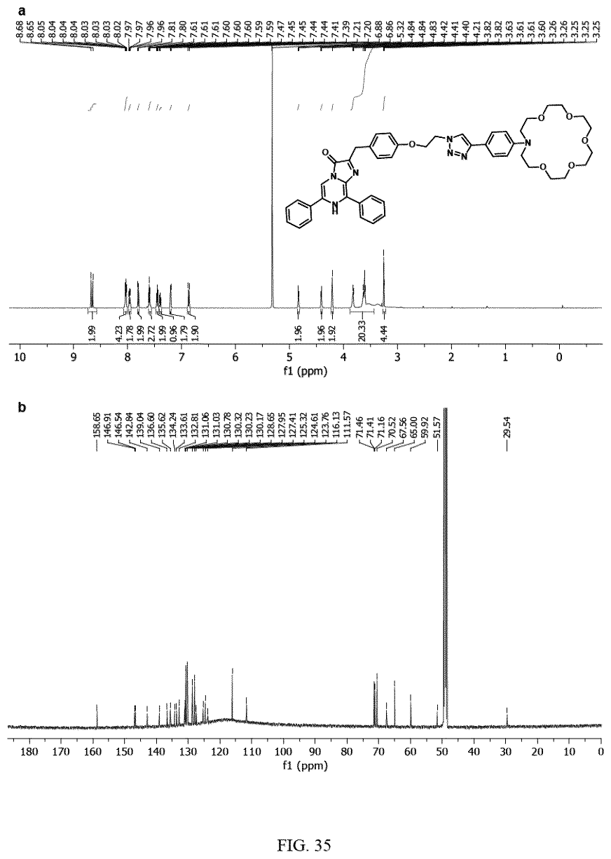

To explore the potential extension of our strategy in incorporating other analyte-binding moieties into luciferins, we synthesized a potassiorin analog with a smaller crown ether ring. The size of the crown ether cavity directly affects its ability to selectively bind specific cations. In this case, we anticipated that the smaller crown ether ring would enhance the complexation with Na⁺, which is smaller than K⁺. Using a similar multi-step synthesis process as potassiorin, we successfully prepared R15-DTZ (FIG. 31), which contains a 15-member-ring aza-crown-ether moiety. To assess the performance of R15-DTZ, we conducted assays using BREP and observed strong bioluminescence quenching induced by Na⁺ (FIG. 33, a). The BREP and R15-DTZ combination also exhibited responsiveness to K⁺, but the apparent affinity for K⁺ was approximately 6.5-fold lower than that for Na⁺ (FIG. 33, b). This suggests that the BREP and R15-DTZ pair may be well-suited for detecting extracellular Na⁺, as Na⁺ is more dominant than K⁺ in the extracellular space. Furthermore, these results indicate the potential for future modifications to develop bioluminescent indicators specifically designed for the detection of intracellular Na⁺. Overall, these findings highlight the broad applicability of our strategy in incorporating sensory moieties into luciferins, paving the way for the development of diverse bioluminescent indicators for various analytes.

### Methods

### General Methods and Information

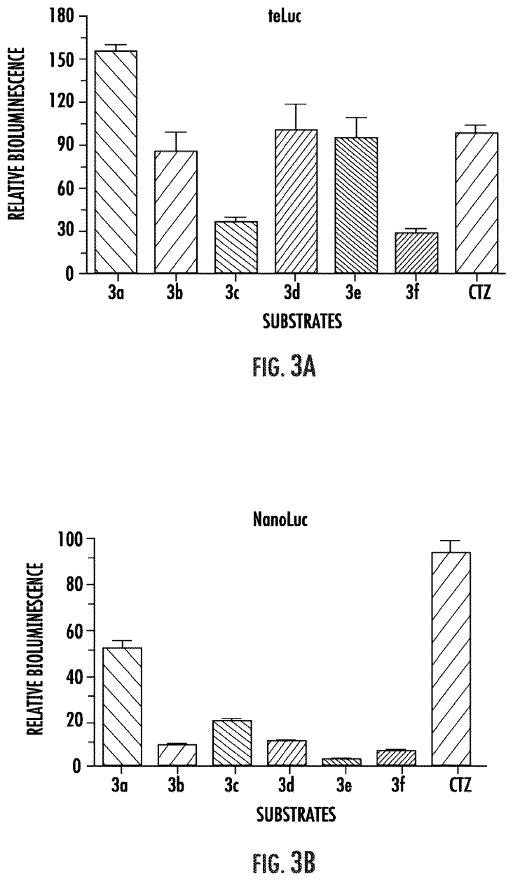

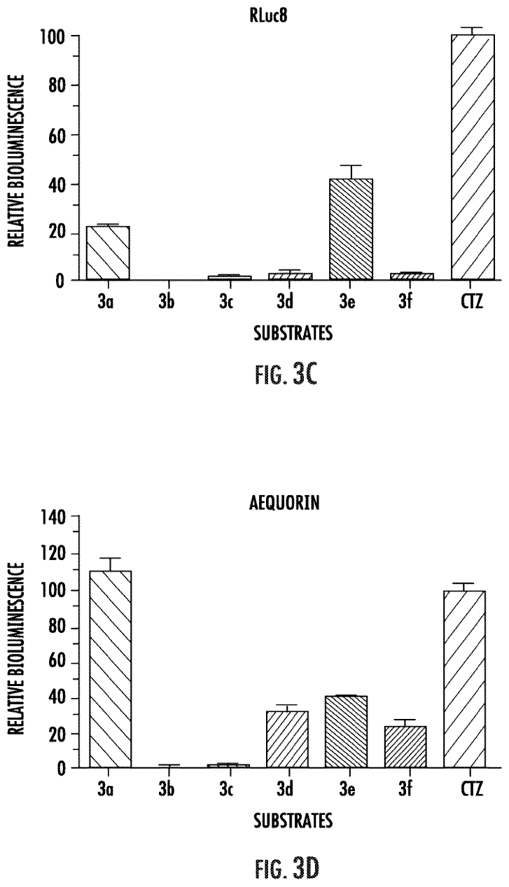

DNA oligos were purchased from either Integrated DNA Technologies or Eurofins Genomics. Restriction enzymes and Phusion High-Fidelity DNA polymerase were purchased from ThermoFisher. Taq DNA polymerase was purchased from New England Biolabs. DNA sequencing was performed by Eurofins Genomics. All animal experiments were conducted following the guidelines and approval (Protocol #4196) of the University of Virginia Institutional Animal Care and Use Committee. BALB/cJ mice (#000651) were obtained from the Jackson Laboratory and housed in a temperature-controlled room (˜23° C.) with a 12-hour light-dark cycle and approximately 50% humidity. At approximately six weeks of age, the mice were randomly assigned to experimental groups, ensuring a balance of both female and male animals. All 1H and ¹³C NMR spectra were collected on a Bruker Avance DRX 600 NMR Spectrometer at the UVA Biomolecular Magnetic Resonance Facility. Chemical shifts (δ) are given with the internal standards: ¹H (7.26 ppm) and ¹³C (77.0 ppm) for CDCl₃; 1H (5.32) for CD₂Cl₂, and ¹³C (49.00 ppm) for CD₃OD. Splitting patterns are reported as s (singlet), d (doublet), t (triplet), dd (doublet of doublets), and m (multiplet). Coupling constants (J) are reported in Hz. Synthetic schemes and compound numbering information are shown in FIG. 3. NMR spectra for key compounds are presented in FIGS. 12 and 13. ChatGPT was utilized to paraphrase sentences and correct grammatical errors in this manuscript.

### Synthesis of 3-(4-(2-azidoethoxy)phenyl)-1,1-diethoxypropan-2-one (6)

First, compound 2 was synthesized as a white powder from 4-benzyloxybenzyl alcohol (1) following a published procedure. Next, 4 was synthesized from 2 in two steps using a previous procedure. Subsequently, 6 was obtained as a colorless oil from 4 in two steps as previously reported.

### Synthesis of N-(4-ethynylphenyl) aza-18-crown-6 (10)

Compound 10 was synthesized from N-phenyl diethanolamine (7) through several steps. First, 8 was obtained as a white solid according to the literature. Then, 9 was prepared from 8 using a published procedure. In the next step, compound 10, which was reported previously, was obtained from 9 using a revised procedure. Briefly, 9 (600 mg, 1.63 mmol, 1 equiv.) was dissolved in 10 mL dry methanol and stirred with dry K₂CO₃ (899 mg, 6.52 mmol, 4 equiv.) in a 100 mL round bottom flask at room temperature. Then, 588 μL of dimethyl (1-diazo-2-oxopropyl) phosphonate (753 mg, 3.9 mmol, 2.4 equiv.) was added. The reaction mixture was stirred overnight at room temperature and monitored by thin-layer chromatography (TLC). After the reaction neared completion, 50 mL ddH₂O was added to the reaction mixture. Then, the resulting mixture was subjected to three extractions with 50 mL of ethyl acetate each time. The organic layers were combined, dried over anhydrous Na₂SO₄, filtered, and concentrated under vacuum. The resulting residue was purified by silica column chromatography using an elution solvent mixture of ethyl acetate and hexane (3:10, gradually shifting to pure ethyl acetate). This yielded compound 10 as a white solid (473 mg, 1.3 mmol, 80% yield).

### Synthesis of 3-(4-(2-(4-(4-(1,4,7,10,13-pentaoxa-16-azacyclooctadecan-16-yl)phenyl)-1H-1,2,3-triazol-1-yl) ethoxy)phenyl)-1,1-diethoxypropan-2-one (11)

Compound 10 (180 mg, 0.585 mmol) and compound 6 (236 mg, 0.585 mmol) were suspended in a mixture of ddH₂O (4 mL) and tert-butyl alcohol (4 mL). Then, sodium ascorbate (12.8 mg, 0.0585 mmol, freshly prepared as a 5 mL solution in ddH₂O) was added, followed by copper (II) sulfate pentahydrate (1 mg, 0.00585 mmol, pre-dissolved in 5 mL ddH₂O). The resulting heterogeneous mixture was vigorously stirred overnight until it cleared, and TLC analysis confirmed the complete consumption of the reactants. The reaction mixture was subsequently diluted with 20 mL of ddH₂O and extracted three times with 20 mL of ethyl acetate. The organic layers were combined, washed with 20 mL of brine, dried over Na₂SO₄, filtered, and concentrated under vacuum. The resulting residue was purified by silica column chromatography using an elution solvent mixture of ethyl acetate and hexane (3:10, gradually shifting to pure ethyl acetate). This yielded 300 mg (77% yield) of pure product as a sticky light-yellow oil. ¹H NMR-(600 MHz, CDCl₃): δ 7.78 (s, 1H), 7.62 (d, J=8.8 Hz, 2H), 7.10 (d, J=8.7 Hz, 2H), 6.82-6.81 (m, 2H), 6.70 (d, J=8.9 Hz, 2H), 4.74-4.72 (m, 2H), 4.59 (s, 1H), 4.34-4.32 (m, 2H), 3.80 (s, 2H), 3.71-3.63 (m, 26H), 3.55-3.50 (m, 2H), 1.21 (t, J=14 Hz, 6H). ¹³C NMR (151 MHz, CDCl₃): δ 203.3, 156.8, 148.2, 147.8, 130.9, 126.9, 119.1, 114.6, 111.8 102.2, 70.7, 70.65, 70.62, 70.60, 68.6, 66.5, 63.3, 51.2, 49.6, 42.6, 15.0. HR-MS (C₃₅H₅₀N₄O₉): [M+H]⁺, calcd: 671.3651. found: 671.3653.

### Synthesis of Potassiorin (13)

3,5-diphenylpyrazin-2-amine (12) was prepared from commercially available 3,5-dibromopyrazin-2-amine according to a previously described procedure. Next, a solution of compound 12 (25 mg, 0.1 mmol, 1 equiv.) and compound 11 (134 mg, 0.2 mmol, 2 equiv.) in 5 mL degassed 1,4-dioxane was prepared. Then, 0.5 mL of 6 N HCl (30 equiv.) was added to the solution. The resulting mixture was stirred at 80° C. in a sealed pressure tube (MilliporeSigma, Cat. #Z568767) for 12 hours. Afterward, the reaction was cooled down to room temperature, and the solvent was removed under vacuum. The residue was dissolved in a 1 mL solution of methanol and water (1:1, v/v). The resulting mixture was filtered through a 0.22 μm polytetrafluoroethylene (PTFE) membrane filter and further purified with a Waters Prep 150 liquid chromatography coupled with an SQ Detector 2 mass spectrometer. An XBridge BEH Amide/Phenyl OBD Prep Column (130 Å, 5 μm, 30 mm×150 mm) was used along with a gradient elution of acetonitrile and water (1:99 to 90:10) at a flow rate of 20 mL/min. The fractions containing the desired product were combined and subjected to lyophilization, resulting in the potassiorin compound as an orange powder (8 mg, 0.01 mmol, 10% yield). 1H-NMR (600 MHz, CD₂Cl₂) δ 8.66 (d, J=20.3 Hz, 2H), 8.05-8.02 (m, 4H), 7.97 (dd, J=1.5, 7.5 Hz, 2H), 7.80 (d, J=8.7 Hz, 2H), 7.61-7.59 (m, 3H), 7.47-7.44 (m, 2H), 7.41-7.38 (m, 1H), 7.25 (d, J=8.7, 2H), 6.91 (d, J=8.7, 2H), 4.84 (t, J=9.9 Hz, 2H), 4.41 (t, J=10 Hz, 2H), 4.21 (s, 1H), 3.82-3.60 (m, 20H), 3.26-3.25 (m, 4H). ¹³C NMR (151 MHz, CD₃OD) δ 158.7, 146.9, 146.5, 142.8, 139.0, 136.6, 135.6, 134.2, 133.6, 132.8, 131.1, 131.0, 130.8, 130.3, 130.2, 130.1, 128.7, 127.9, 127.4, 125.3, 124.6, 123.8, 116.1, 111.6, 71.5, 71.4, 71.2, 70.5, 67.6, 65.0, 59.9, 51.6, 29.5. HR-MS (C₄₇H₅₁N₇O₇): [M+H]⁺, calcd: 826.3923. found: 826.3892.

### Library Construction and Screening

To create libraries with random mutations, the BREP gene was amplified from our previously described pcDNA3-BREP plasmid (Addgene, Cat. #172337) using Taq DNA polymerase under a previously established error-prone condition. The resulting mutated genes were then subcloned into a pBAD/His B plasmid using Gibson assembly. E. coli DH10B competent cells were transformed by electroporation and plated on 2xYT agar supplemented with 100 μg/mL ampicillin and 0.2% (w/v) L-arabinose. After overnight incubation at 37° C., approximately 200 μL of 25 μM potassiorin was sprayed onto the colonies on each plate. BLI was performed using a UVP BioSpectrum dark box, a Computar Motorized ZOOM lens (M6Z1212MP3), and a Teledyne Photometrics Evolve 16 EMCCD camera. Colonies displaying strong bioluminescence were selected and cultured individually in wells of 96-well plates containing 1 mL of 2xYT media supplemented with 100 μg/mL ampicillin and 0.2% (w/v) L-arabinose. After shaking at 37° C. for 20 hours, bacterial cells were pelleted by centrifugation and lysed using 500 μL of Thermo Fisher Bacterial Protein Extraction Reagent (B-PER). In the initial screening stage, the bioluminescence of E. coli lysates was measured in the presence of potassiorin under two KCl concentrations: 0 mM and 150 mM. For this, 30 μL of each cell lysate was diluted with 50 μL of MOPS buffer (10 mM, pH 7.4) containing either 0 mM or 240 mM KCl, resulting in final KCl concentrations of 0 mM and 150 mM, respectively. Meanwhile, potassiorin (5 mM) dissolved in a premade stock solution (ethanol: 1,2-propanediol=1:1 (v/v), supplemented with 0.88 mg/mL L-ascorbic acid) was diluted to 100 μM using the MOPS buffer containing no KCl or 150 mM KCl. A 20 μL aliquot of the potassiorin solution was dispensed into each well of a microplate using an automated dispenser on a CLARIOstar Microplate Reader (BMG Labtech). After a 1-second shake, the bioluminescence spectra ranging from 450 nm to 700 nm were recorded using the plate reader equipped with a red-sensitive PMT. Mutants that exhibited extreme K-dependent bioluminescence changes were chosen for subsequent screening, which focused on their resistance to Na⁺. A similar procedure as described above was employed to test the bioluminescence responses of the mutants to 30 mM NaCl versus no NaCl. The mutant showing the highest response to K⁺ and the lowest response to Na⁺ was chosen as the template for the next screening round. To construct the focused library targeting residues 233, 260, and 261, oligos containing NNK degenerate codons (where N=A, T, G, or C and K=G or T) were utilized to amplify three short gene fragments. Subsequently, Gibson assembly was employed to fuse these fragments with the predigested pBAD/His B plasmid. The remaining steps involved in library screening were identical to the procedures described above.

### Protein Purification and In Vitro Assays

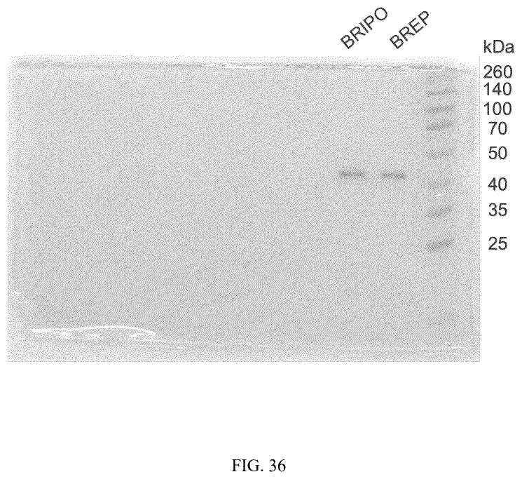

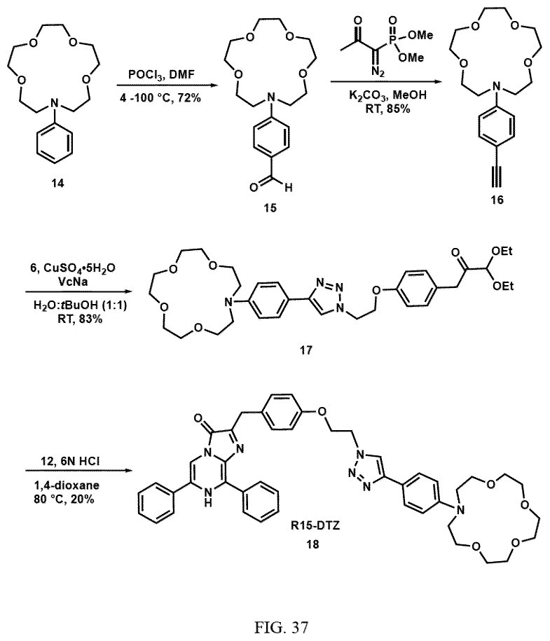

Recombinant proteins BREP and BRIPO were expressed and purified following a previous procedure, and the purity was verified using SDS-PAGE (FIG. 36). The purified proteins were diluted in MOPS buffer to a final concentration of 200 nM, with either 0 mM or 300 mM KCl. For the initial assay, 50 μL of each protein dilution was added to the wells of a 96-well plate. Then, 50 μL of potassiorin (50 μM) in MOPS buffer was dispensed into each well, resulting in final KCl concentrations of 0 mM or 150 mM. The bioluminescence spectra were recorded using the CLARIOstar Microplate Reader. For the K⁺ or Na⁺ concentration dependence assays, 50 μL of MOPS buffer containing 200 nM purified proteins and a specific concentration of NaCl or KCl was added to the wells of a 96-well plate. Then, 50 μL of potassiorin (50 M) in MOPS buffer was injected into each well, establishing final ion concentrations ranging from 1800 mM to 0.5 mM. The bioluminescence spectra were recorded and the intensity values at 590 nm were plotted against ion concentrations. Data was fit using the one-site binding model in GraphPad Prism 9. For the ion selectivity assays, 50 μL of MOPS buffer containing 200 nM purified BRIPO and a specific metal ion was added to the wells of a 96-well plate. Then, 50 μL of potassiorin (50 μM) in MOPS buffer was injected into each well. Bioluminescence was recorded, with metal ions supplied as follows: NaCl (15 mM), ZnCl₂ (10 μM), CaCl₂) (2 mM), KCl (150 mM), MgCl₂ (2 mM), MnCl₂ (10 μM), FeCl₂ (10 μM), CuCl₂ (100 nM). Two additional CuCl₂ concentrations (1 μM and 10 μM) were tested in the presence of either potassiorin or DTZ. For the substrate concentration dependence assays, 50 μL of MOPS buffer containing 200 nM purified BRIPO and either no or 300 mM KCl was added to the wells of a 96-well plate. Various volumes of MOPS buffer and potassiorin solutions were injected into each well to achieve potassiorin concentrations ranging from 25 μM to 0.5 μM. After a 1-second shake, the bioluminescence of each well was recorded. The data was fit using the Michaelis-Menten nonlinear regression function in GraphPad Prism 9. To determine the bioluminescence quantum yields (QY) of BRIPO and potassiorin, an excess of enzyme was employed. The purified protein was diluted to a final concentration of 1.7 μM using a 0.1 M Tris buffer (pH 8.0) with or without 150 mM KCl. Subsequently, 50 μL of the diluted protein was dispensed into the wells of a 96-well plate. Then, 50 μL of potassiorin, diluted in the same buffer at a concentration of 10 nM, was added to each well to achieve a final enzyme and luciferin concentration of 0.85 μM and 5 nM, respectively. Bioluminescence from each well was promptly recorded at 1-second intervals for 1000 seconds. The bioluminescence intensity diminished to insignificance by the end of the measurement. The area under the intensity curve was computed to represent the relative total photon emission. Measurements were conducted concurrently with the FLuc enzyme (MilliporeSigma, Cat. #SRE0045) and D-luciferin (Glod Biotechnology, Cat. #LUCK100) under previously established conditions, utilizing the reported QY of 0.41 as the reference to calculate the QYs for BRIPO and potassiorin in the presence and absence of 150 mM KCl.

### Characterization in Mammalian Cell Lines

The BRIPO gene was amplified from the pBAD plasmid and inserted into a pcDNA3 vector, resulting in pcDNA3-BRIPO. HEK 293T cells (ATCC, Cat. #CRL-3216) were cultured in DMEM supplemented with 10% fetal bovine serum (FBS). A HEK 293T cell line stably expressing mTrekl and the α1H subunit of Ca_{V}3.2, provided by Dr. Paula Barrett (University of Virginia), was cultured in DMEM supplemented with 10% FBS, 1% penicillin/streptomycin, 0.4 μg/mL puromycin, and 400 μg/mL G418. The generation and characterization of this cell line were previously described. Both types of cells were transfected using a previously described procedure. Imaging was conducted 2 to 3 days later. For the K⁺ efflux experiments, cells were rinsed three times with a lab-made cell imaging buffer (15 mM D-glucose, 0.1 mM sodium pyruvate, 0.49 mM MgCl₂, 2 mM CaCl₂, 0.4 mM MgSO₄, 0.44 mM KH₂PO₄, 5.3 mM KCl, 4.2 mM NaHCO₃, 0.34 mM Na₂HPO₄, 138 mM NaCl, 10 mM HEPES, pH 7.2). Cells were then maintained in this buffer supplemented with 50 μM potassiorin or DTZ for bioluminescence. Time-lapse imaging was performed using an inverted Leica DMi8 microscope equipped with a Photometrics Prime 95B Scientific CMOS camera. The imaging settings included a 40x oil immersion objective lens (NA 1.2), no filter cube, 2×2 camera binning, 10 s exposure with no interval, camera sensor temperature of −20° C., and 12-bit high-sensitivity mode. For HEK 293T cells, nigericin (10 mM), bumetanide (10 mM), and ouabain (10 mM) ethanol stocks were diluted in the imaging buffer mentioned above to achieve final concentrations of 20 μM, 10 μM, and 10 μM, respectively. For the stable HEK 293T cells, arachidonic acid (10 mM) ethanol stock was diluted in the same imaging buffer to a final concentration of 20 μM. For the K⁺ influx experiments involving the HEK 293T cells, the procedures remained identical except for the utilization of a lab-made high-K⁺ cell imaging buffer (15 mM D-glucose, 0.1 mM sodium pyruvate, 0.49 mM MgCl₂, 2 mM CaCl₂), 0.4 mM MgSO₄, 0.44 mM KH₂PO₄, 200 mM KCl, 4.2 mM NaHCO₃, 0.34 mM Na₂HPO₄, 10 mM HEPES, pH 7.2). Acquired images were processed using the Fiji version of ImageJ 1.53e as described. Data were plotted, and statistical analysis was performed using GraphPad Prism 9. The baselines caused by substrate decay were corrected according to the previously described procedure.

### Viral Preparation and Characterization in Primary Mouse Neurons

The BRIPO gene was amplified from the corresponding pcDNA3 plasmid and subsequently inserted into a pAAV-hSyn vector, resulting in the creation of pAAV-hSyn-BRIPO. AAVs carrying the BRIPO gene were prepared using our previously reported procedure. The obtained AAV titers were about 1×10¹³ GC/mL. Following preparation, the AAVs were aliquoted and stored at −80° C. for long-term preservation. Primary mouse neurons were prepared as described. Neurons were seeded on 35 mm glass-bottom dishes coated with poly-D-lysine, supplemented with 2 mL NbActiv4 medium (BrainBits). The culture was maintained at 37° C. with 5% CO₂. On the fourth day post-plating, half of the medium was changed to fresh NbActiv4. On the same day, 3 μL of the BRIPO virus and 1 μL of 1 M HEPES (pH 7.4) were added to each 35 mm culture dish. Neurons were imaged four days post-transduction. The growth medium was carefully replaced with 0.5 mL of the cell imaging buffer supplemented with 100 μM of potassiorin or DTZ before imaging. Time-lapse imaging was performed under the same settings described in the mammalian cell imaging section. During time-lapse imaging, glutamate dissolved in the above-mentioned imaging buffer was added to the dish at a final concentration of 1 mM. Image processing and data analysis were identical to the procedure described in the mammalian cell imaging section.

### Imaging of K⁺ Dynamics in Live Mice

For each BALB/cJ mouse, 500 nL of AAV was delivered to both sides of the hippocampus (AP −1.7, ML±1.2, DV −1.5) and cortex (AP −1.7, ML±1.2, DV −0.5) via intracranial stereotactic injection at a flow rate of 100 nL/min. The needle remained in the brain for additional 5 min after the infusion was complete, and the wound was sealed with surgical adhesive. Two to three weeks after the virus injection, potassiorin (5 mM) or DTZ (15 mM) pre-dissolved in a stock solution (ethanol:1,2-propanediol=1:1 (v/v), supplemented with 0.88 mg/mL L-ascorbic acid) was diluted in saline to a concentration of 25 μM. The mice were anesthetized, and 500 nL of the diluted compound was injected into the virus infusion sites. Time-lapse imaging was then performed using a UVP BioSpectrum dark box, a Computer Motorized ZOOM lens (M6Z1212MP3), and a Teledyne Photometrics Evolve 16 camera. The instrumental settings were as follows: camera sensor gain of 3, PMT gain of 600, 2×2 binning, camera sensor temperature of −20° C., and 10 s exposure time with no interval. The ZOOM lens was set to be 100% open, 0% zoom, and 0% focus. The mice were positioned 20 cm away from the front of the lens without an emission filter. During the time-lapse imaging, the mice were briefly removed from the dark box and intracranially injected with 500 nL of glutamate (10 mM in saline) into the middle of the virus injection sites (AP-0.7, ML 0, DV-1.0) at a flow rate of 250 nL/min. The mice were immediately placed back in the dark box for subsequent imaging. Data analysis followed the same procedure described in the mammalian cell imaging section.

### An Enhanced Red Bioluminescent Indicator for Responsive Detection of Physiological Calcium Dynamics in Cells and Mice

### Introduction

Ca²⁺ is among the most widely recognized secondary messengers, intricately linked to numerous biological processes. Over the years, fluorescent calcium indicators have been developed into powerful research tools, enabling researchers to monitor Ca²⁺ dynamics with high spatiotemporal resolution at both the cellular and subcellular levels. Since Ca²⁺ influx serves as a proxy for neuron activation, these Ca²⁺ indicators have been extensively applied to in vivo animal imaging, becoming indispensable for studying brain functions. However, their use is limited by inherent challenges associated with the requirement for excitation photons. These include phototoxicity to biological samples, interference with photon-sensitive biological processes, and incompatibility with optogenetic tools. Additionally, observing neuronal activity in brain regions often necessitates invasive procedures, such as the implantation of imaging windows, gradient-index (GRIN) lenses, or optical fibers.

Bioluminescence is a naturally occurring photon-generation mechanism in which a luciferase enzyme uses molecular oxygen to oxidize a luciferin substrate, producing light. The amount of light generated through bioluminescence is relatively low and is generally considered safe for biological systems. Although bioluminescence imaging (BLI) has limited spatiotemporal resolution, it complements the shortcomings of fluorescence imaging. Unlike fluorescence imaging, bioluminescence imaging does not require external excitation, making it inherently compatible with photon-sensitive biological processes and optogenetic methods. Furthermore, bioluminescence creates a self-illuminating light source, making it particularly well-suited for minimally invasive imaging in deep tissues.

NanoLuc (NLuc) is an engineered luciferase derived from the functional subunit of Oplophorus luciferase (OLuc). It features a miniaturized protein size (˜19.2 kDa) and high brightness, making it an excellent choice for bioluminescence imaging. Over the past decade, NanoLuc and its derivatives have been effectively developed into numerous bioluminescent indicators. Given the pivotal role of Ca²⁺ in neuronal activity and the potential of bioluminescent imaging for minimally invasive monitoring of specific neuronal populations in animals, developing bioluminescent indicators for Ca²⁺ has been a highly sought-after topic in the field. Despite the progress, only a few bioluminescent Ca²⁺ indicators are suitable for in vivo animal imaging since it requires the superior transmission of long-wavelength red light in mammalian tissues. Our previously reported indicator, BRIC (bioluminescent red indicator for Ca²⁺), stands out due to its superior brightness, response magnitude, and red-shifted emission, making it one of the top choices for in vivo BLI. However, despite the progress, the performance of bioluminescent Ca²⁺ indicators still lags significantly behind fluorescent Ca²⁺ sensors.

Herein, we report the engineering of an enhanced Ca²⁺ sensor, eBRIC, derived from the original BRIC through three rounds of directed evolution. Compared to BRIC, eBRIC exhibits a significantly improved Ca²⁺ response in vitro, as well as in cultured mammalian cell lines and primary neurons. To extend its application to live animal models, we used eBRIC to record Ca²⁺ dynamics in the somatosensory cortex and BLA regions in live mice, showcasing the ability of eBRIC for capturing the real-time activity of a neuronal population in a physiologically relevant context.

### Methods

General Information. All animal studies were carried out per the Institutional Animal Care and Use Committees' approvals at the University of Virginia (Protocol #4196) and Cornell (Protocol #2015-0029). BALB/cJ mice (#000651) and C57BL/6 J mice (#000664) were procured from the Jackson Laboratory and bred and maintained under standard conditions. DTZ and sDTZ luciferins were chemically synthesized according to the previously reported procedures.

Engineering and in Vitro Characterization of eBRIC. Random mutations were introduced into BRIC in a pBAD/HisB vector via error-prone PCRs, and the resultant libraries were screened as previously described except for the following modification. After preparing E. coli cell lysates, 5 μL of the supernatant was mixed with 185 μL of a buffer providing 65 nM free Ca²⁺ (30 mM MOPS, 100 mM KCl, 8.75 mM EGTA, 1.25 mM CaEGTA, pH 7.2) or another buffer providing 1.35 μM free Ca²⁺ (30 mM MOPS, 100 mM KCl, 5 mM EGTA, 5 mM CaEGTA, pH 7.2). 10 μL of DTZ solution (500 μM) was dispensed into each well using a reagent injector in a BMG Labtech CLARIOstar Plus microplate reader, resulting in a final DTZ concentration of 25 μM. The bioluminescence spectrum of each well was recorded from 450 to 700 nm with 10 nm intervals. Mutants exhibiting both high brightness and Ca²⁺ responsiveness were chosen for further analysis. Through three rounds of directed evolution, eBRIC was developed. The expression, purification, concentration determination, and in vitro characterization of the eBRIC protein were conducted following the established procedures used for BRIC.

Characterization of eBRIC in Mammalian Cell Lines. The gene for eBRIC was cloned into pcDNA3 to create pcDNA3-eBRIC, with pcDNA3-BRIC utilized for comparison. Next, 3 μg of plasmid DNA was used to transfect HeLa cells (ATCC, Cat. #CCL-2) in each 35-mm culture dish. After overnight incubation at 37° C. in a 5% CO2 incubator, cells were rinsed twice with DPBS (no Ca²⁺ and Mg2), followed by a 20-minute incubation in DPBS before imaging using an inverted Leica DMi8 microscope equipped with a Photometrics Prime 95B Scientific CMOS camera and controlled by Leica LAS X (Version 3.5.7) software. Bioluminescence was initiated by exchanging DPBS with fresh DPBS containing 100 μM DTZ. Imaging parameters included a 40× oil immersion objective lens (NA 1.2), no filter cube, 2×2 camera binning, 1 s exposure with 5 s intervals, camera sensor temperature set at −20° C., and camera in 12-bit high sensitivity mode. Histamine, dissolved in DPBS, was introduced at a final concentration of 20 μM during the time-lapse imaging. Image processing and data analysis were conducted following established protocols. The experiments with HEK 293T cells (ATCC, Cat. #CRL-3216) were performed similarly, except that a luminescence imaging buffer (0.49 mM MgCl₂, 2 mM CaCl₂), 0.4 mM MgSO₄, 0.44 mM KH₂PO₄, 5.3 mM KCl, 4.2 mM NaHCO₃, 0.34 mM Na₂HPO₄, 138 mM NaCl, 10 mM HEPES pH 7.2, 15 mM D-glucose, and 0.1 mM sodium pyruvate) was used instead of DPBS. In addition, the images were acquired with 2 s exposure and 25 s intervals. Acetylcholine (Thermo Scientific, Cat. #AC159170050), dissolved in the luminescence imaging buffer, was introduced at a final concentration of 10 μM.

Characterization of eBRIC in Primary Mouse Neurons. The production of adeno-associated viruses (AAVs), neuron preparation, transduction, and culture procedures were conducted following established protocols. Neurons expressing eBRIC and BRIC were assessed on the fifth day post-transduction with the corresponding AAVs. Prior to imaging, the growth medium was substituted with 1.6 mL of the luminescence imaging buffer supplemented with 100 M DTZ. During time-lapse imaging, 0.42 mL of high K⁺ stimulation buffer (0.49 mM MgCl₂, 2 mM CaCl₂), 0.4 mM MgSO₄, 0.44 mM KH₂PO₄, 143.2 mM KCl, 4.2 mM NaHCO₃, 0.34 mM Na₂HPO₄, 10 mM HEPES pH 7.2, 15 mM D-glucose, and 0.1 mM sodium pyruvate) was applied to depolarize neurons. The imaging setup and data analysis were consistent with the previous section, except for an exposure time of 2 s with 3 s intervals.

Surgery Preparation and Bioluminescence Recording of Somatosensory Cortex Ca²⁺ Dynamics in Mice. C57BL/6J mice (n=6) were anesthetized with isoflurane and secured in a stereotaxic frame with a heating pad. A 4-mm craniotomy was made above the whisker barrel cortex (AP: −1 to −1.5 mm; ML: 3.5 to 4.0 mm), as described previously. AAV-hSyn-eBRIC (5×10¹³ GC/mL) was injected into four sites (250 nL each at 2 nL/s) using a glass micropipette. A PDMS cranial window was placed over the cortex and sealed with Vetbond (3M), followed by skull reinforcement with Metabond (Parkell) and attachment of a titanium headplate. Mice recovered for at least three weeks before imaging. For imaging, mice were anesthetized with isoflurane (4% induction, ˜0.5% maintenance). Laser speckle imaging confirmed functional activation in the whisker barrel cortex. Expression of eBRIC was verified using a custom-built two-photon microscope (920 nm excitation) under 4× and 20× (ZEISS W Plan-Apochromat lens, NA 1.0) objectives. For intravenous DTZ delivery, the tail was disinfected with 70% ethanol and warmed. A catheter (26G needle with polyurethane intravascular tubing (BTPU-027) prefilled with 10 U/mL heparinized saline) was inserted into a lateral tail vein, and 10 μL of saline was flushed to prevent clotting. The catheter was secured with medical tape, and the mouse was head-fixed onto a stereotaxic stage via the implanted headplate. Three needle electrodes were inserted into the whisker pad and connected to an Isolated Pulse Stimulator (Model 2100, A-M Systems); one served as ground, and the others targeted distinct whisker pad regions. After locating the area of viral expression via two-photon fluorescence, the excitation laser was blocked, and bioluminescent emission was captured using the microscope detectors with a 645/65 filter on photomultiplier tube (Hamamatsu, H10770B-50) with a home-built preamplifier (I to V gain 2×10⁵ Ohms; ˜10 MHz bandwidth). A 5 mL injection buffer for DTZ was made by dissolving 1.25 g (2-hydroxypropyl)-β-cyclodextrin (HP-β-CD) and 1 mL PEG-400 in ˜3 mL normal saline. DTZ was then dissolved in this buffer to a final concentration of 2.5 mM. DTZ was infused into mice at 25 μL/min via a syringe pump (KD Scientific). Electrical stimulation (5 Hz, 5 s, 2 mA and 4 mA) was delivered through the electrodes. Following imaging, electrodes and catheter were removed, the tail was disinfected, and the mouse was returned to its home cage.

BLI of BLA Ca²⁺ Dynamics in Awake Mice. 1 μL of AAV-hSyn-eBRIC (5×10¹³ GC/mL) or an equivalent amount of AAV-hSyn-BREP was bilaterally injected into the BLA brain region (coordinates relative to Bregma: AP-3.4, ML+1.25, DV-4.9) 19 of 8-week-old BALB/cJ mice. BLI was conducted three weeks post-viral administration. Before imaging, each awake mouse received a tail vein injection of 100 μL of sDTZ (25 mM) in normal saline. The mouse was then secured in a Narishige plastic mouse head holder (SRP-AM2) for imaging. BLI was performed using a UVP BioSpectrum dark box, a Computar Motorized ZOOM lens (M6Z1212MP3), and a Photometrics Evolve 16 EMCCD camera. The camera settings included an EM gain of 1000, 8×8 binning, an exposure time of 100 ms without intervals, and a sensor temperature of −70° C. The mice were positioned 27 cm from the lens. Each experimental session consisted of a 100 s acclimation period for the animals, followed by 13 footshock trials. Each trial involved a 0.8-mA electric footshock lasting 1 s, with 40 s intervals between shocks, administered using an A-M Systems 2100 isolated pulse stimulator. Image processing and data analysis were performed according to an established procedure.

### Results

Engineering and Characterization of eBRIC In Vitro

BRIC is a bioluminescent Ca²⁺: indicator generated by inserting the Ca²⁺-sensory calmodulin (CaM) and M13 moieties into a NanoLuc-derived teLuc luciferase, linked to the red fluorescent protein mScarlet-I. The indicator has more than half of its emission above 600 nm due to bioluminescent resonance energy transfer (BRET) from the Ca²⁺-regulated luciferase to the red fluorescent protein. In vitro, it showed an excellent 6.5-fold ΔBL/BL₀ response. Although BRIC has been established as a benchmark for BLI of Ca²⁺, its cellular responses require further improvement.

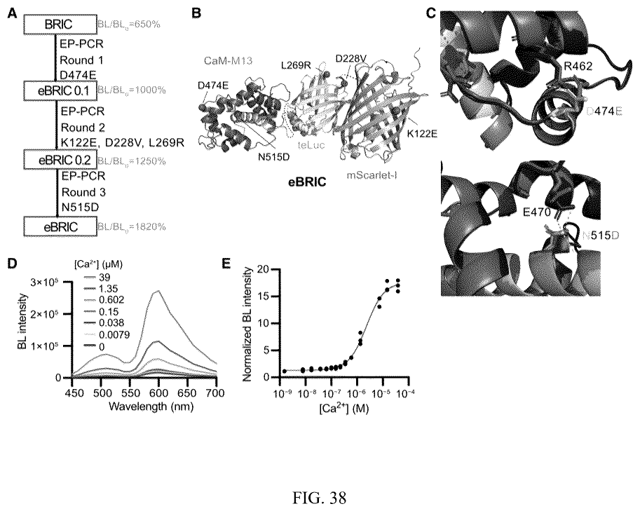

Built upon the success of BRIC, we aimed to further improve its responsiveness to physiological Ca²⁺ fluctuations. To achieve this, we conducted random mutagenesis on BRIC and screened the resulting libraries using 65 nM and 1.35 μM of free Ca²⁺, which differs from previous screening approaches that used 0 and 39 μM of free Ca²⁺. This new range of Ca²⁺: concentrations was chosen because it better matches the cytosolic Ca²⁺ concentrations of mammalian cells at rest and excited states. Through three rounds of directed evolution, we successfully generated a drastically enhanced BRIC (eBRIC) variant with an 18.2-fold Ca²⁺-included bioluminescence increase in cell lysates (FIG. 38, A). We also measured the brightness of BRIC and eBRIC in both the Ca²⁺-free (off) state and the Ca²⁺-bound (on) states using bacterial lysates. Compared to BRIC, eBRIC displayed reduced brightness in the Ca²⁺-free state but enhanced brightness in the Ca²⁺-bound state (data not shown).

During this process, five mutations were identified: two in teLuc (L269R and D228V), one in mScarlet-I (K122E), and two in the CaM-M13 region (D474E and N155D) (FIG. 38,B). Notably, the two mutations in the CaM-M13 region are primarily responsible for the observed response enhancement (FIG. 38, A). Although it is challenging to precisely understand the underlying mechanisms, based on our ColabFold-generated models, residue 474 is involved in a hydrogen bond with Arg462, bridging two a-helices close to two Ca²⁺ binding sites. Residue 155 forms a hydrogen bond with E470, facilitating interactions between M13 and CaM (FIG. 38, C). Presumably, these mutations help maximize the structural and activity differences between the Ca²⁺-free and Ca²⁺-bound states of the sensor.

We further performed spectroscopic characterization using purified proteins. From BRIC to eBRIC, the response between 65 nM and 1.35 μM of Ca²⁺ increased from 1.7-fold to 4.6-fold, while the overall Ca²⁺-induced change between 0 and 39 μM of free Ca²⁺ increased from 6.5-fold to ˜17-fold (FIG. 38, D). The apparent dissociation constant (K_{d}) also increased from 133 nM to 2.3 μM (FIG. 38, E). Collectively, these results indicate that eBRIC is a high-quality Ca²⁺ sensor with the potential for sensitively detecting Ca²⁺ changes in the physiologically relevant concentration range.

Characterization of eBRIC in Cultured Mammalian Cells and Primary Neurons

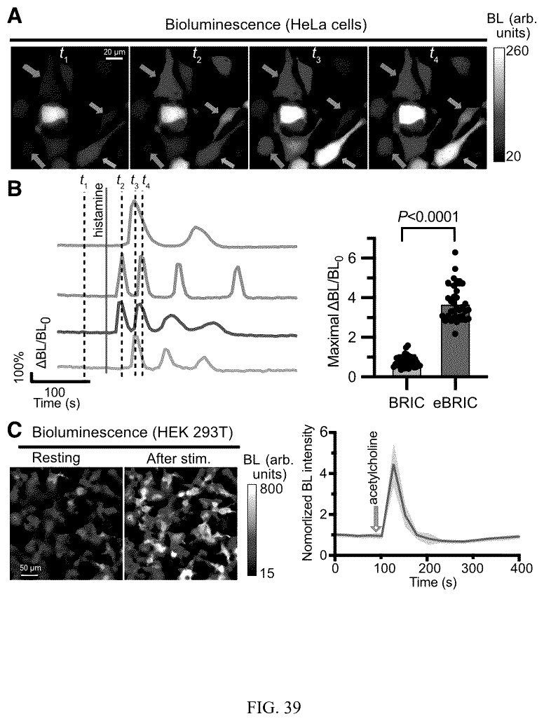

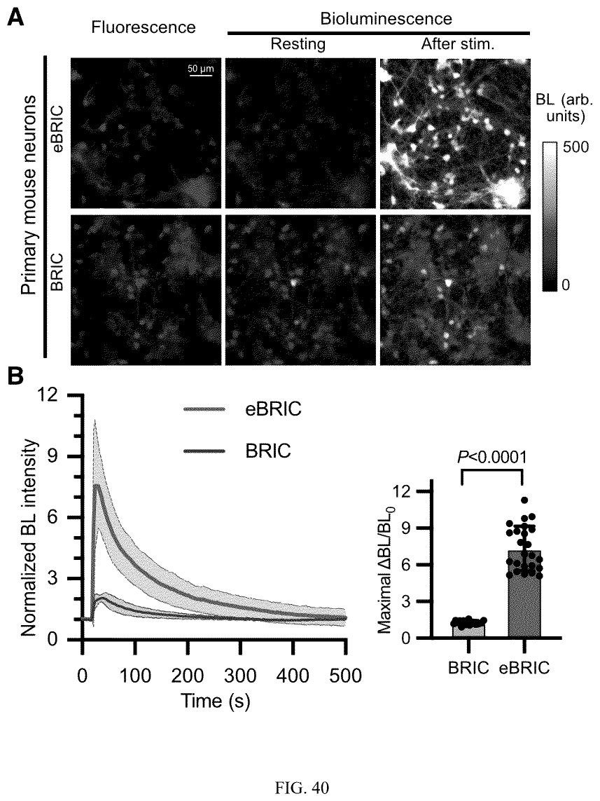

Next, we tested eBRIC for imaging Ca²⁺ dynamics in Hela cells. 100 μM DTZ was added to initiate bioluminescence. In eBRIC-cells, the stimulation with 20 μM histamine resulted in robust bioluminescence oscillations, which indicates Ca²⁺ oscillations (FIG. 39A,B). Comparing this to BRIC at identical conditions, the magnitude of the change (ΔBL/BL₀) was nearly 5-fold higher. To test whether eBRIC is functional in other cell types, we expressed eBRIC in HEK 293T cells and stimulated them with 10 μM of acetylcholine. We observed a robust 3.5-fold (ΔBL/BL₀) bioluminescence increase, indicating a successful detection of acetylcholine-induced Ca²⁺ increases in the cytol (FIG. 39 C). Finally, we compared eBRIC and BRIC in primary mouse neurons. high K⁺ depolarization induced a 7.3-fold increase (ΔBL/BL₀) in eBRIC bioluminescence, resulting in a 5.6-fold increase in responsiveness from BRIC (FIG. 40).

The brightness of bioluminescent indicators is a critical parameter for effective imaging. We compared the brightness of eBRIC to BRIC in both transfected HeLa cells and AAV-transduced primary mouse neurons (not shown). In Hela cells, we measured brightness in both the Ca²⁺-free (off) state and the Ca²⁺-bound (on) state following histamine stimulation. eBRIC exhibited reduced brightness in the Ca²⁺-free state but demonstrated approximately twice the brightness of BRIC after stimulation. A similar trend was observed in primary neurons. To further evaluate the performance of eBRIC in mammalian systems, we measured its bioluminescence emission spectrum in HEK 293T cells (not shown). The spectral profile closely resembled that of eBRIC in E. coli lysates and as a purified protein, suggesting that expression in mammalian cells does not alter the BRET efficiency.

Taken together, eBRIC has proven to be a highly effective bioluminescent indicator for detecting physiologically relevant Ca²⁺ changes in living cells.

### In Vivo BLI of Ca²⁺ Dynamics in Live Mice

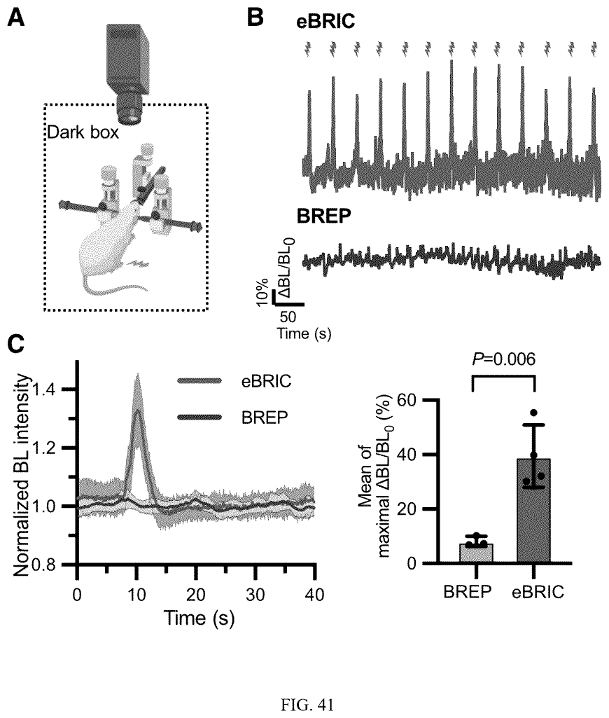

To further demonstrate the use of eBRIC for in vivo imaging of the activity of neuronal ensembles in awake animals, we used an AAV carrying the eBRIC gene to infect neurons in the basolateral amygdala (BLA) region of the brains of live mice. We paired eBRIC with our recently developed water-soluble sDTZ luciferin28 for bioluminescence whole-animal imaging. The BLA is known to play a crucial role in generating fear-associated behaviors, and aversive stimuli such as footshock are expected to activate neurons in this region. Prior to imaging, each mouse received a tail vein injection of 100 μL sDTZ (25 mM) dissolved in normal saline. During imaging, mice were subjected to repeated 1-second footshock stimulations at 0.8 mA, delivered at 40-second intervals. As expected, we observed a significant and consistent increase in bioluminescence in the eBRIC group in response to footshocks (FIG. 41). As a negative control, we generated mice expressing BREP, a Ca²⁺-insensitive bioluminescent protein, and observed minimal to no changes in bioluminescence under electric footshock stimulation. The combination of eBRIC and sDTZ allowed digitalization at a frequency of 10 Hz, which has not been achieved in previous studies. Compared to prior experiments with BRIC under the same stimulation condition, the response magnitude of eBRIC has nearly doubled. These findings confirm that eBRIC serves as a powerful tool for tracking the activity of neuronal populations with improved temporal resolution and sensitivity.

### Discussion

Bioluminescent Ca²⁺ indicators hold great promise as tools for functional imaging of bioactivities. In contrast to fluorescent indicators, bioluminescent indicators provide the advantage of being less invasive. However, a key limitation of existing bioluminescent Ca²⁺: indicators is their relatively low responsiveness. Here we developed an enhanced bioluminescent Ca²⁺ indicator (eBRIC) based on BRIC by using a physiological Ca²⁺ concentration range during library screening. We successfully optimized eBRIC to exhibit significantly improved performance. In vitro characterization and experiments in mammalian cells and animals revealed that eBRIC demonstrates increased sensitivity to physiological Ca²⁺ levels.

Although the Ka of eBRIC has increased to 2.3 μM, it exhibits a higher fold of bioluminescence changes within the range of 65 nM to 1.35 μM Ca²⁺. Subsequently, eBRIC demonstrated favorable performance in both cellular and animal models. Compared to Orange CaMBIs, we gained 7.4- to 18-fold increase in Ca²⁺ responsiveness using the new eBRIC Ca²⁺ sensor in physiologically stimulated live cells. Compared to our previously reported BRIC sensor, eBRIC is 4.5- to 5.6-fold more responsive. As BRIC has been set as a benchmark for bioluminescent Ca²⁺ indicators. eBRIC signifies a substantial advancement beyond BRIC. Indeed, the combination of eBRIC with our recently developed luciferin substrate, sDTZ, expands the capability of functional neuronal imaging. The successful demonstration of minimally invasive, video-rate imaging of Ca²⁺ activity in a defined brain region in awake mice represents a significant milestone, underscoring the potential of eBRIC as a powerful tool for investigating dynamic processes in living organisms.

While eBRIC demonstrates clear performance gains over its predecessor, the substantial response enhancement observed in cultured cells translated to a more modest improvement in the footshock-induced BLA activation paradigm in animals. The underlying reasons for this discrepancy are not fully understood, but may include factors such as limitations in substrate availability, measurement noise, hemodynamic and temperature effects, and variability in sensor expression or folding. It is worth noting that similar reductions in performance have been frequently observed with other fluorescent protein-based biosensors when transitioning from in vitro to in vivo settings. Thus, to overcome these limitations, more frequent iterative testing between in vitro and in vivo systems—or approaches that enable direct engineering and optimization of indicators within animal models—may be necessary. Additionally, while eBRIC produces greater bioluminescence than BRIC in the Ca²⁺-bound state, its reduced brightness in the Ca²⁺-free state may limit the signal-to-noise ratio under basal conditions. When paired with sDTZ, which can be administered at a concentration of 25 mM, eBRIC enabled time-lapse imaging at 10 Hz. However, in vivo applications remain limited by the inherent trade-off between absolute signal intensity and achievable spatial-temporal resolution-particularly in deep tissue, where photon attenuation significantly reduces sensitivity. Although the temporal resolution represents an improvement, it may still fall short for accurately capturing rapid, transient Ca²⁺ dynamics.

We also recorded bioluminescence from live-animal brain activity using a microscope setup with the excitation light blocked. These results demonstrate that eBRIC can reliably report neural activity evoked by somatosensory stimulation in the cortex of a mouse implanted with a chronic cranial window and systemically administered substrate. Notably, we observed clear neural responses to single whisker pad shocks in individual trials, underscoring the high signal-to-noise ratio of eBRIC in vivo. Although a two-photon microscope was used to confirm sensor expression, the actual bioluminescence recordings do not require such specialized instrumentation. Since fluorescence microscopes are widely accessible, researchers can readily adapt this recording format for their own studies. Additionally, we used a syringe pump to infuse the DTZ substrate continuously, which provided more sustained bioluminescence signals compared to traditional bolus injections—a strategy that may prove useful for extended imaging sessions.

In parallel with our work, a recent preprint reported a promising bioluminescent Ca²⁺ indicator called CaBLAM, which is based on a newly developed SSLuc luciferase. In validation experiments, CaBLAM demonstrated robust and impressive responses. Unlike eBRIC, CaBLAM emits in the blue range and does not incorporate a red fluorescent protein for BRET-based red shifting. As a result, imaging was performed using a wide-field microscope equipped with an EMCCD camera, under no external illumination, targeting superficial cortical regions accessible through cranial windows. Nonetheless, eBRIC should also be compatible with such imaging setups. Future studies directly comparing eBRIC and CaBLAM would be valuable for benchmarking their relative strengths and limitations under various experimental conditions and for guiding the development of next-generation bioluminescent indicators.

In conclusion, the development of eBRIC signifies a leap forward in bioluminescent imaging technology. Its high sensitivity, enhanced response magnitude, and compatibility with less invasive imaging techniques make it a powerful tool for investigating dynamic processes in living organisms. We anticipate that eBRIC will become an invaluable asset for researchers, enabling new studies on biological processes, neuronal dynamics, and disease mechanisms.

### PEGylated ATP-Independent Luciferins for Non-Invasive High-Sensitivity High-Speed Bioluminescence Imaging

As discussed throughout herein, bioluminescence imaging (BLI), which utilizes luciferase-catalyzed oxidation of luciferins for photon production, is a powerful, non-invasive method for monitoring biological processes in animal models. Compared to fluorescence imaging, BLI avoids issues such as photobleaching and phototoxicity and enables sensitive signal monitoring in deep tissues with high signal-to-background ratios. Compared to alternative in vivo imaging modalities such as magnetic resonance imaging (MRI) and positron emission tomography (PET), BLI offers superior spatiotemporal resolution, cost-effectiveness, convenience, and the absence of radioactive contrast agents. When combined with bioluminescent indicators, BLI can track specific bioactivities, making it a popular imaging technique for both basic and preclinical research.

Firefly luciferase (FLuc), which utilizes D-luciferin as its substrate, is a widely adopted bioluminescent reporter due to its long-wavelength light emission and the substrate's chemical stability and water solubility. Research has developed FLuc and D-luciferin derivatives offering enhanced properties such as higher brightness, more red-shifted emission, and orthogonal reactivity. Of particular note is the Akaluc luciferase coupled with the AkaLumine luciferin (also known as TokeOni), recognized as a leading benchmark for deep-tissue sensitivity. However, these luciferases and their derived bioluminescent indicators inherently depend on ATP, a crucial energy source and signaling molecule, for their operation.

Conversely, the oxidation of coelenterazine (CTZ) luciferin by marine luciferases is ATP-independent. In 2012, Promega introduced NanoLuc, a marine luciferase mutant, known for its high photon production with the synthetic luciferin named furimazine, small molecular size, remarkable enzyme stability, and flexibility for split and domain insertion. Despite its popularity, NanoLuc faces challenges for in vivo BLI, such as limited tissue penetration of the emitted blue photons, low substrate stability and solubility, and inadequate luciferin entry to the brain. Recent studies partially addressed these concerns by developing additional CTZ analogs and NanoLuc mutants, or by fusing luciferases to long-wavelength-emitting fluorescent proteins (FPs) for redder emission. Furthermore, NanoLuc and its derived luciferases, as disclosed herein, have been effectively transformed into bioluminescent indicators, enabling successful imaging of Ca²⁺ and K⁺ dynamics in live mice. Despite recent advancements, the in vivo sensitivity of NanoLuc-derived luciferase-luciferin pairs still falls short compared to FLuc-derived benchmark reporters. This limitation continues to hinder the full potential of NanoLuc-derived bioluminescent indicators. To address this challenge, there is a pressing need for further research to systematically improve the in vivo capabilities of NanoLuc-derived luciferases and indicators.

Here, we present a PEGylation method to enhance the auto-oxidation resistance and water solubility of marine luciferase substrates. We showcased the versatility of this approach using three luciferins, resulting in a series of new prosubstrates with remarkably increased solubility. Given the challenges of brain BLI due to the blood-brain barrier (BBB) limiting luciferin entry and the need for capturing rapid neuronal activity we focused on characterizing these prosubstrates using mouse models expressing luciferases in the brain. We observed drastic bioluminescence enhancements with these water-soluble luciferins that are deliverable at a safe concentration of 25 mM. Notably, we identified the brightest luciferase-luciferin combination, which surpassed the brightness of Akaluc and AkaLumine, allowing high-speed video-rate imaging of freely moving mice with brain-expressed luciferase. Furthermore, we validated the substantial brightness enhancement in non-brain tissues using one of the novel luciferins and a mouse model with liver luciferase expression. Collectively, these findings strongly indicate that PEGylation of marine luciferase substrates represents a versatile approach to significantly enhance the sensitivity of in vivo BLI.

### Results

### PEGylation of DTZ and Assessment of its Solubility and Auto-Oxidation Resistance

We previously developed a NanoLuc variant, teLuc, that produced bright bioluminescence peaking around 500 nm when combined with a DTZ substrate. More recently, we refined DTZ to create a prosubstrate, ETZ, which could be administered in higher doses and activated in vivo by nonspecific esterase, enhancing in vivo bioluminescence. In parallel, other researchers introduced luciferin variants such as HFz and FFz improving water solubility and brightness in non-brain tissues. However, the solubility improvements from these prior studies were only modest, and high in vivo brightness still required the use of high doses of organic cosolvents or surfactants, which could induce organ toxicity.

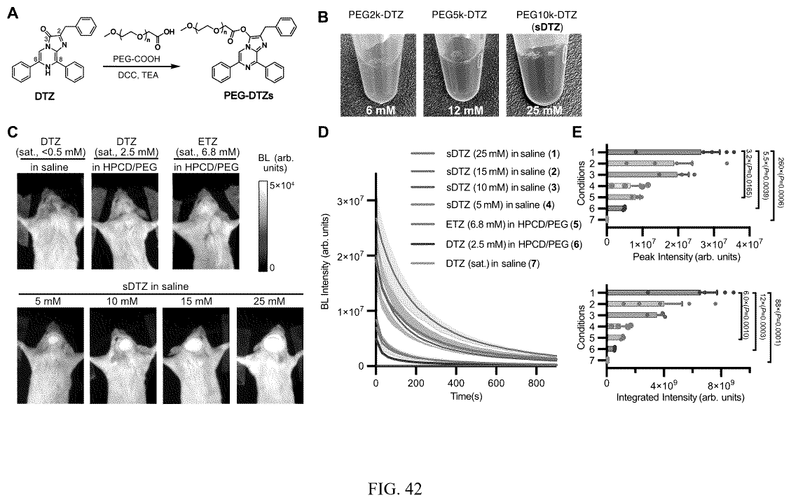

We aimed to significantly enhance the water solubility of marine luciferase substrates, enabling high-dose delivery via aqueous solutions. Polyethylene glycol (PEG) conjugation is a commonly employed method for improving the solubility of hydrophobic drugs. Using DTZ as our model compound, we investigated PEGylation at the C3 carbonyl group of the DTZ imidazopyrazine ring (FIG. 42, A). We expect PEGylation at this site to create caged substrates, but upon injection into animals, nonspecific esterase would quickly remove the PEGylation, releasing free DTZ luciferin.

By conducting condensation reactions of DTZ with methoxy PEG carboxylic acids of varying molecular weights (˜2000, 5000, and 10000 Da), we obtained the polymer-luciferin conjugates (PEG2 k-DTZ, PEG5 k-DTZ, and PEG10 k-DTZ) and determined their maximum solubility in normal saline (pH 7.2) to be approximately 6, 12, and 25 mM, respectively (FIG. 42, B). Given that PEG10 k-DTZ exhibited the highest solubility, we renamed it sDTZ and focused our subsequent experiments on this compound. To provide a basis for comparison, we conducted additional tests to assess the solubility of DTZ in normal saline supplemented with 25 mM PEG10 k. The solubility was found to be between 1 and 2 mM. This observation suggests that the enhanced solubility of sDTZ in normal saline is attributed to the intramolecular ester conjugation with the polymeric PEG chain. Furthermore, we investigated the solubility of sDTZ in mouse serum, and the results indicated a range between 20 and 25 mM.

CTZ and its analogs have the tendency to undergo slow oxidation in the absence of luciferases, and this oxidation reaction relies on the C3 carbonyl group of the DTZ imidazopyrazine ring.⁷ However, since the C3 position in sDTZ is masked, we anticipate that it will exhibit increased resistance to auto-oxidation. To investigate this, we compared the remaining bioluminescence activity of sDTZ and DTZ compounds under various storage conditions (e.g., as solids at room temperature and as neutral aqueous solutions at 4° C., −20° C., and −80° C.). Higher bioluminescence activity was consistently observed with sDTZ across all tested conditions. Despite the improvement, we still observed auto-oxidation of sDTZ, indicating that the ester linkage is susceptible to hydrolysis by water. This can occur both in solution and in the solid state, through absorption of atmospheric moisture. We further utilized thin layer chromatography (TLC) to directly examine ester linkage cleavage. The results revealed that in aqueous solution at 4° C., the hydrolysis of sDTZ commenced within a few hours and was nearly completed in a day.

### Brightness Enhancement for Brain and Liver Imaging, and Toxicity Evaluation

As reported herein, we developed an optimized fusion protein named BREP by combining teLuc with the red fluorescent protein (RFP) mScarlet-I. BREP, when paired with DTZ, emits about 60% of its total emission above 600 nm, positioning it as a highly effective luciferase for deep-tissue BLI. Replacing DTZ with sDTZ is not anticipated to alter the emission profile, because when sDTZ is administered in vivo, it will undergo hydrolysis to convert into DTZ. We generated adeno-associated viruses (AAVs) harboring the BREP gene under the control of the human synapsin I (hSyn) promoter and introduced them into the hippocampal neurons of mice through stereotactic injection. Two to three weeks later, the mice with BREP expressed in the brain were examined under anesthesia in a dark box with an EMCCD camera. We conducted a pilot study to evaluate different delivery methods, including intravenous, intraperitoneal, and retro-orbital injections, to administer 100 μL of 25 mM sDTZ solutions. The highest brightness was achieved when the luciferin was delivered via the tail vein. As a result, we utilized tail vein injection in all subsequent studies.

We subsequently evaluated sDTZ for enhancing BLI brightness using the mouse model. Experimenting with four different sDTZ concentrations revealed that higher luciferin concentrations led to increased bioluminescence intensity (FIG. 42, D). The brightest signal occurred with the maximal sDTZ concentration (25 mM) in normal saline. The signal decreased over time and around 7% of the peak intensity was retained after 15 min (FIG. 42, E). In contrast, DTZ saturated in saline had a peak intensity 260-fold lower and a 15-min integrated intensity 88-fold lower (FIG. 42, F). Strong bioluminescence was observed when DTZ and ETZ were dissolved in a formulation containing 25% (w/v) 2-hydroxypropyl-β-cyclodextrin (HPCD) and 20% (v/v) PEG-400, which allowed the maximal final concentrations of DTZ and ETZ to be approximately 2.5 mM and 6.8 mM, respectively. However, the sDTZ in saline group still had a peak intensity 5.5-fold and 3.2-fold higher than DTZ and ETZ, respectively. In addition, the integrated bioluminescence signal over 15 min for the sDTZ in saline group was approximately 12-fold and 6-fold higher than the DTZ and ETZ in HPCD/PEG groups, respectively (FIG. 42, F). Collectively, the results indicate that sDTZ enables efficient in vivo luciferin delivery using normal saline, significantly enhancing sensitivity for brain BLI in mice.

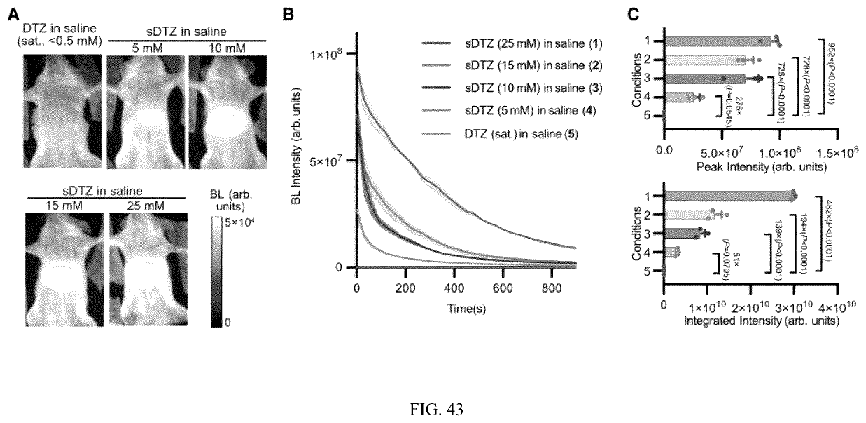

To explore bioluminescence improvement in different tissues, we introduced BREP luciferase into mouse livers using AAVs with a hepatocyte-specific thyroxine-binding globulin (TBG) promoter. Mice were administered different concentrations of sDTZ before imaging. The bioluminescence intensity increased with higher sDTZ concentrations, exceeding that of DTZ in saline by at least two orders of magnitude (FIG. 43). The 25 mM sDTZ concentration resulted in the highest intensity, with nearly a 1000-fold peak intensity increase and a 482-fold integrated intensity increase over the first 15 min. These findings align with brain imaging results, confirming substantial sensitivity enhancement by sDTZ in other tissue types.

After determining 25 mM sDTZ as optimal for achieving maximum sensitivity, we assessed organ toxicity with five consecutive daily injections. At the end of the experiment, mice were sacrificed, and organs were extracted and examined with hematoxylin and eosin (H&E) staining. No apparent organ toxicity was found, suggesting sDTZ's safety for animal imaging.

### PEGylation of Alternative Luciferins and Brain Brightness Evaluation

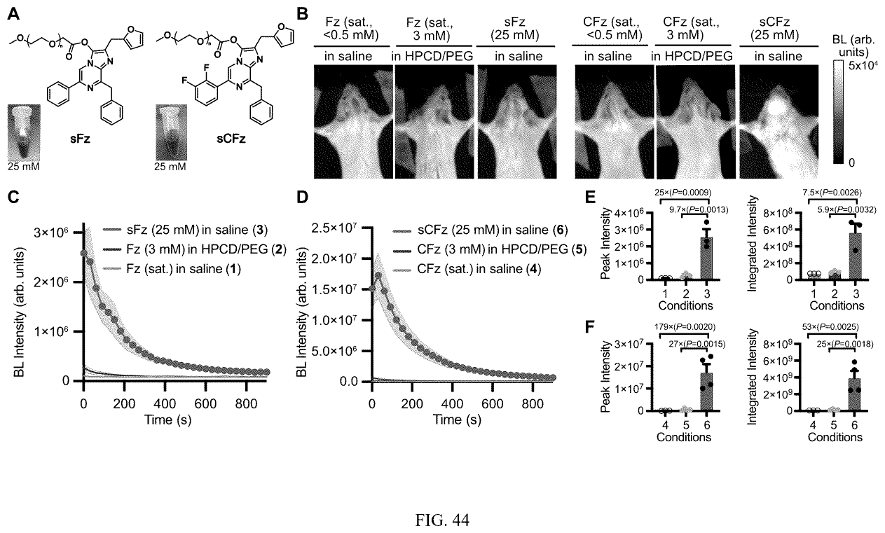

After achieving success with sDTZ, we extended the PEGylation method to other luciferins. Specifically, we focused on two crucial ones: furimazine (Fz), the original NanoLuc substrate, and CFz, a recently developed NanoLuc substrate with improved BBB penetration. We introduced a methoxy PEG chain (MW˜10,000) to create sFz and sCFz, respectively (FIG. 44, A). As anticipated, the PEGylation of Fz and CFz increased their resistance to auto-oxidation. To assess sFz and sCFz for in vivo BLI, we used AAVs to express the Antares luciferase, a fusion of NanoLuc to two copies of CyOFPI, in the hippocampal region of the mouse brain. We injected sFz or sCFz (25 mM in normal saline) via the tail vein and compared results with Fz or CFz saturated in the HPCD/PEG buffer mentioned earlier, as well as normal saline. Both sFz and sCFz significantly enhanced brightness (FIG. 44, B-F). Particularly, sCFz led to a 179-fold and 27-fold peak bioluminescence increase compared to CFz in normal saline and the HPCD/PEG buffer, respectively. Moreover, the sCFz group showed approximately 5-6-fold brighter signals than sFz (FIG. 44, EF), consistent with CFz's better BBB permeability than Fz. These findings indicate that PEGylation can improve solubility and greatly enhance in vivo BLI sensitivity across different luciferins.

### Identification of the Optimal Luciferase-Luciferin Combination and High-Speed Video-Rate Brain Imaging of Freely Moving Mice

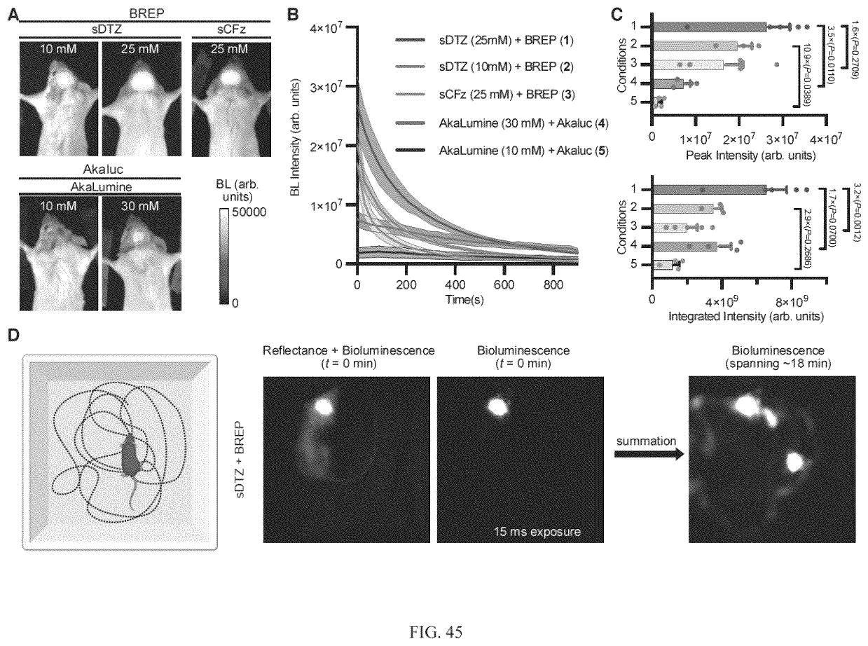

Based on our data, both BREP-sDTZ and Antares-sCFz combinations displayed bright bioluminescence, with BREP-sDTZ being roughly twice as bright as Antares-sCFz (FIGS. 43, F and 45, F). Considering the cross-reactivity of teLuc in BREP towards NanoLuc substrates, we further examined the bioluminescence of mice with hippocampal BREP expression after delivering sCFz (FIG. 45, A). The BREP-sCFz mice exhibited strong bioluminescence, with intensity below the BREP-sDTZ mice (FIG. 45, B, C). Although the sample size was inadequate to statistically distinguish the peak intensities, BREP-sCFz displayed a quicker bioluminescence decay and lower 15-min integrated bioluminescence that is statistically significant compared to BREP-sDTZ (FIG. 45, B,C). These findings indicate that the BREP-sDTZ combination exhibits the most promise among the tested ATP-independent luciferase-luciferin pairs.

In addition, we compared BREP-sDTZ with the ATP-dependent luciferase-luciferin pair, Akaluc-AkaLumine. All luciferins were administered intravenously in normal saline to mice with hippocampal luciferase expression. At 10 mM luciferin concentrations, the peak intensities of BREP mice were approximately 11 times brighter than Akaluc mice (FIG. 45, A-C). With 25 mM sDTZ and 30 mM AkaLumine, BREP remained 3.5 times brighter than Akaluc. Despite displaying a faster bioluminescence decay, BREP-sDTZ consistently exhibited greater brightness in the initial 10 min, indicating its suitability for imaging applications demanding high sensitivity (FIG. 45B).

Given the excellent performance of sDTZ, we conducted high-speed video-rate BLI of freely moving mice expressing BREP in the hippocampus (FIG. 45, D). The bioluminescence glow from the brain was so bright and even visible in brightfield imaging at the beginning of the experiment. By alternating between brightfield and bioluminescence imaging, we successfully captured mouse movement and brain bioluminescence for over 18 min using a 15-ms exposure. Previous research has demonstrated the capability to monitor Akaluc and Antares signals in moving mouse brains using AkaLumine or CFz. Despite variations in protocols and equipment, our study achieved enhanced temporal resolution by utilizing BREP-sDTZ, even in unshaved mice.

### Discussion

Disclosed herein is the development of a PEGylation method for modifying marine luciferase substrates. By attaching PEG chains to these substrates, their water-solubility was greatly enhanced, allowing for effective in vivo luciferin administration using a simple and non-toxic normal saline solution. The resulting luciferase-luciferin pairs exhibited notable increases in brightness, surpassing established benchmarks such as Akaluc-AkaLumine and Antares-CFz. This research tackles critical obstacles in the field, achieving optimal sensitivity with ATP-independent luciferase reporters and enabling high-speed video-rate imaging of freely moving animals.

It is worth noting that the PEGylated substrates are expected to undergo rapid de-caging upon injection into animals, similar to our previous study on ETZ which confirmed the labile nature of esterification through the C3 carbonyl group in vivo. The presence of PEG chains is not anticipated to affect BBB crossing, except for enabling the delivery of higher doses in aqueous buffers. Additionally, the elimination of organic cosolvents or surfactants from the luciferin delivery solution enhances the biocompatibility of BLI. Previous studies have often utilized Poloxamer 407 (P-407) as a delivery vehicle for similar luciferins, but multiple injections have been shown to induce liver, kidney, and heart toxicity, atherosclerosis, and weight loss in mice.

Furthermore, the extension of the PEGylation technology to multiple luciferins signifies a significant advancement. CFz has recently been reported as a promising luciferin for brain imaging. In our research, we achieved a remarkable 179-fold and 27-fold increase in bioluminescence brightness using sCFz compared to CFz saturated in normal saline or an HPCD/PEG buffer (FIG. 44). This study highlights the versatility of PEGylation with various CTZ analogs, expanding the potential applications and research opportunities in this field.

These PEGylated ATP-independent luciferins demonstrated increased resistance to auto-oxidation compared to their parental counterparts, due to the presence of a caged carbonyl group that is essential for the oxidation reaction. However, it is crucial to acknowledge that the ester linkage in these PEGylated luciferins is relatively labile, as evidenced by hydrolysis observed both in vitro and in vivo. While rapid in vivo hydrolysis is advantageous for bioluminescence, in vitro hydrolysis can lead to substrate auto-oxidation and degradation before use. For long-term storage, we recommend storing these PEGylated luciferins in the solid format at temperatures of −20° C. or −80° C. Prior to conducting experiments, it is important to dissolve the solids in a neutral aqueous solution, as the presence of base or acid can catalyze ester hydrolysis. Once the solution is prepared, it remains usable for only a few hours on ice or a few days at −20° C. or −80° C. In contrast, ATP-dependent luciferins such as AkaLumine exhibit greater chemical stability, as they remain inactive until activated by ATP. This distinction is important to consider in terms of handling and storage requirements for these two categories of luciferins.

Our animal validation experiments were centered on brain imaging, considering the challenges posed by the BBB and the essential requirement for high temporal resolution to capture rapid neuronal activities. Compared to the Akaluc-AkaLumine pair, we here gained 3.5- to 10.9-fold bioluminescence brightness increase using ATP-independent BREP-sDTZ (FIG. 45, C). The discovery of BREP-sDTZ as the most luminous luciferase-luciferin combination opens up new possibilities for accurate and sensitive imaging of targets in live organisms. The advantages of BREP-sDTZ would be particularly notable in scenarios where the detection of a small number of cells is beneficial, such as in developmental biology and cancer metastasis research. Furthermore, by combining these new luciferins with rapidly expanding bioluminescent biosensors, our study provides a pathway for noninvasively capturing fast biological dynamics, including neuronal activity. Additionally, ATP-independent bioluminescence systems described here offer the benefit of minimal disruption to biological processes, as ATP is an endogenous energy and signaling molecule. These systems also exhibit effective functionality in extracellular spaces and biological fluids like serum and urine, where ATP concentrations are typically low. Conversely, Akaluc-AkaLumine relies on ATP for bioluminescence and does not function effectively under these conditions.

When it comes to generating sustained signals, Akaluc-AkaLumine continues to outperform ATP-independent bioluminescence systems, including BREP-sDTZ (FIG. 45, B). Further research is necessary to address this limitation and continue making progress from the work presented here. Additionally, we can envision the fast signal decay of BREP-sDTZ becoming an advantage under certain circumstances. For example, because it is often challenging to spectrally separate bioluminescent reporters in vivo, BLI with multiple luciferases may require sequential injection and imaging using orthogonal luciferins. Since the signals of BREP-sDTZ would largely dissipate after 1-2 hours following luciferin delivery, it would allow quicker imaging of the second reporter.

In this disclosure, luciferins were delivered into mice via tail vein injection, as our main objective was to achieve the highest peak bioluminescence intensity. Compared to intraperitoneal injection, tail vein injection yielded approximately 100-fold higher bioluminescence, albeit with a shorter signal duration. Moreover, retro-orbital injection demonstrated promising results, as it produced bright bioluminescence and appeared to have a longer signal duration compared to tail vein injection. We intend to further investigate and explore this approach in our future studies.

This study further illustrates that the application of PEGylated luciferins extends to non-brain tissues. The use of sDTZ allowed for high-sensitivity BLI of the liver, indicating that the novel tools may facilitate the study of various organ-specific processes, disease models, and therapeutic interventions in non-brain systems.

In summary, this disclosure has introduced a PEGylation method to enhance a range of marine luciferase substrates, achieving the highest bioluminescence sensitivity with these novel caged PEGylated luciferins. These new tools provide researchers with unparalleled sensitivity, resolution, and versatility, serving as invaluable assets for furthering our comprehension of biological processes and disease mechanisms.

### Methods

Ethical Statement. All animal studies were carried out per the University of Virginia Institutional Animal Care and Use Committee's approval (Protocol #4196) and guidelines. BALB/cJ mice (#000651) and C57BL/6 J mice (#000664) were procured from the Jackson Laboratory and bred under standard conditions. The mice were housed in a temperature-controlled environment (˜23° C.) with a 12 h/12 h light-dark cycle and ˜50% humidity. Animals were randomly allocated to experimental groups to ensure a mix of male and female subjects.

Chemical Synthesis and Characterization. Methoxy PEG acetic acids (PEG-COOH) with molecular weights of ˜2,000 Da (Cat. #A3138), ˜5,000 Da (Cat. #A3071), and ˜10,000 Da (Cat. #A3081) were procured from JenKem Technology. DTZ, Fz, and CFz were synthesized following established procedures,^{17,20,21} and purified using a Waters Prep 150/SQ Detector 2 LC-MS Purification System with MassLynx software (Version 4.2) and an XBridge BEH Amide/Phenyl OBD Prep Column (130 Å, 5 μm, 30 mm×150 mm). For the conjugation reaction, methoxy PEG acetic acid (0.02 mmol, 1 equiv.) and DCC (12.4 mg, 0.06 mmol, 3 equiv.) were combined in a dried and argon-purged two-neck 50 mL round-bottom flask. A mixture of anhydrous dichloromethane (10 mL) and triethylamine (4.4 μL, 0.06 mmol, 3 equiv.) was slowly added while stirring at room temperature for 20 minutes. Then, DTZ, Fz, or CFz (0.024 mmol, 1.2 equiv.) was swiftly introduced into the reaction mixture. The reaction was stirred under argon for an additional hour and monitored by TLC (DCM/methanol=10:1). Upon completion, the reaction mixture was concentrated in vacuo, and the residue was washed with ethyl ether and purified by silica gel chromatography (DCM/methanol=15:1, v/v). The resulting compound was collected, concentrated, dissolved in 5 mL ddH2O, filtered through VWR Grade 415 qualitative filter paper, and lyophilized to obtain sDTZ, sFz, or sCFz as solids using a 12-port Labconco freeze dryer with an Edwards RV3 vacuum pump. The purity of the compounds was reverified using TLC (Fig. S13), and the recovery yields based on the input of PEG-COOH were approximately 80%. 1H-NMR spectra were recorded on a Bruker Avance III 600 MHz NMR spectrometer with Bruker TopSpin IconNMR software (Version 3.5pl4) and analyzed using MestReNova software (Version 12.0.3). Figs. S14 to S17 display the ¹H NMR spectra for DTZ, Fz and CFz (dissolved in Methanol-d₄), as well as sDTZ, sFz, sCFz and the starting material, PEG10 k (dissolved in D20). DTZ: ¹H NMR (600 MHz, methanol-d₄) δ 8.10-8.01 (m, 2H), 7.95-7.65 (m, 3H), 7.64-7.54 (m, 3H), 7.49 (t, J=7.5 Hz, 2H), 7.46-7.38 (m, 1H), 7.38-7.21 (m, 4H), 7.15 (t, J=7.4 Hz, 1H), 4.16 (s, 2H). sDTZ: ¹H NMR (600 MHz, D20) δ 8.39 (s, 1H), 8.05-8.00 (m, 3H), 7.64-7.53 (m, 4H), 7.14-6.98 (m, 8H), 4.01 (s, 2H), 3.82 (s, 2H), 3.51-3.40 (m, PEG), 3.18 (s, 3H). Fz: ¹H NMR (600 MHz, methanol-d4) δ 7.77 (s, 1H), 7.68 (d, J=7.4 Hz, 2H), 7.51-7.44 (m, 3H), 7.43-7.40 (m, 2H), 7.38 (dd, J=1.9, 0.9 Hz, 1H), 7.33-7.27 (m, 2H), 7.25-7.21 (m, 1H), 6.32 (s, 1H), 6.10 (s, 1H), 4.43 (s, 2H), 4.19 (s, 2H). sFz: ¹H NMR (600 MHz, D₂O) δ 8.63 (s, 1H), 8.43-8.19 (m, 1H), 8.10 (s, 1H), 7.85-7.80 (m, 3H), 7.50-7.28 (m, 9H), 6.39 (s, 1H), 6.23 (s, 1H), 4.64 (s, 2H), 4.20 (m, 2H), 4.03 (s, 2H), 3.65-3.59 (m, PEG), 3.30 (s, 3H). CFz: ¹H NMR (600 MHz, methanol-d₄) δ 7.87 (s, 1H), 7.53 (s, 1H), 7.44-7.35 (m, 3H), 7.34-7.26 (m, 3H), 7.23 (t, J=7.5 Hz, 1H), 6.32 (s, 1H), 6.10 (s, 1H), 4.41 (s, 2H), 4.19 (s, 2H). sCFz: ¹H NMR (600 MHz, D₂O) δ 8.62 (s, 1H), 8.43-8.37 (m, 2H), 8.10 (s, 1H), 7.47-7.08 (m, 6H), 6.37 (s, 1H), 6.22 (s, 1H), 4.64 (s, 2H), 4.18 (m, 2H), 4.00 (s, 2H), 3.65-3.58 (m, PEG), 3.29 (s, 3H). PEG10 k: ¹H NMR (600 MHz, D₂O) δ 4.10 (s, 2H), 3.66-3.54 (m, PEG), 3.30 (s, 3H).

Evaluation of auto-oxidation resistance. DTZ and sDTZ were evaluated in two forms: as 100 μM solutions in Tris-HCl buffer (100 mM, pH 7.4) at temperatures of 4, −20, and −80° C., and as powders stored at room temperature in aluminum foil-wrapped microcentrifuge tubes to shield from light exposure. The remaining bioluminescence activity was assessed using purified BREP luciferase. For the assays, solid powders were dissolved in Tris-HCl buffer (100 mM, pH 7.4) to obtain 100 μM solutions. DTZ solutions were directly used, while sDTZ solutions underwent pretreatment with porcine liver esterase (MilliporeSigma, Cat. #E3019) by incubating 100 μL of the sDTZ solution with 1 μL of 50 units/mL enzyme for 15 minutes at room temperature. Subsequently, DTZ or esterase-treated sDTZ were mixed with purified BREP protein to achieve final concentrations of 50 μM luciferin and 10 nM enzyme. Bioluminescence spectra were recorded using a BMG Labtech CLARIOstar Plus microplate reader controlled by BMG Labtech Reader Software (Version 5.70 R2), with results automatically exported to BMG Labtech MARS Data Analysis Software (Version 3.42 R5). A similar procedure was employed to compare sFz and sCFz with Fz and CFz as 100 μM solutions in Tris-HCl buffer (100 mM, pH 7.4) at 4° C.

Toxicity Assessment in Mice. sDTZ (25 mM) was dissolved in normal saline (0.9% NaCl, Baxter, Cat. #G158220), and a daily intraperitoneal injection of 100 μL of the solution was administered to C57BL/6J mice for five consecutive days. Normal saline without other compounds served as the control. Tissues from the brain, heart, liver, lung, kidney, and spleen of the mice were harvested at the end of the fifth day. The UVA Research Histology Core Facility conducted paraffin-embedded tissue sectioning and H&E staining.

BLI of Mice with Hippocampal Luciferases. The process of generating AAVs for neuronal expression of BREP has been detailed previously. To create analogous viral vectors for expressing Akaluc or Antares, the BREP gene in the pAAV-hSyn-BREP transfer plasmid was substituted with the Akaluc or Antares gene, resulting in the pAAV-hSyn-Akaluc or pAAV-hSyn-Antares transfer plasmid for viral packaging. The titers of the purified viral stocks were quantified using qPCR and were subsequently diluted with Dulbecco's Phosphate-Buffered Saline (DPBS) to achieve concentrations of 101+GC/mL before use. 1 μL of the diluted virus was bilaterally injected into the hippocampus (relative to Bregma: AP-1.7, ML+1.2 and DV-1.5) of 8-week-old BALB/cJ mice via intracranial stereotactic injection at a flow rate of 100 nL/min. Mice were imaged two to three weeks later. A procedure described previously was employed to dissolve DTZ or ETZ in a buffer comprising 25% (w/v) HPCD and 20% (v/v) PEG-400, and 5% (w/v) NaHCO₃ was additionally added to facilitate the dissolution of ETZ. Alternatively, the luciferins were simply dissolved in normal saline Right before imaging, mice received a 100 μL intravenous injection of the dissolved luciferins. They were subsequently imaged using a UVP BioSpectrum dark box, a Computar Motorized ZOOM lens (M6Z1212MP3), and a Photometrics Evolve 16 EMCCD camera. The lens adjustments were made through the UVP VisionWorksLS software (Version 8.6), with the aperture set to 100% open, zoom at 0%, and focus to 0%. Micro-Manager (Version 2.0.0) was utilized to control the camera for imaging acquisition. The camera analog gain was set to high, and the EM gain was 500. Other parameters included a camera binning of 2×2, camera temperature maintained at −70° C., and exposure time of 100 ms with acquisitions every 30 s. Mice were anesthetized and positioned 23 cm away from the lens without an emission filter. The images were processed using the Fiji version of ImageJ 2.14. Initial background subtraction from the image stacks was conducted with a rolling ball radius set to 100 pixels. Subsequently, a region of interest (ROI) was defined based on the bioluminescence signal from the mouse brain, and the integrated intensity value over the ROI was extracted for further analysis. Despite software-based background subtraction, residual background was noted in the images. To address this, the ROI was relocated away from the mouse brain region to evaluate the residual background, which was then subtracted from the signals to calculate the integrated bioluminescence intensity (area under the curve). The data was plotted and subjected to statistical analysis using GraphPad Prism (Version 8.4.3).

BLI of Mice with Liver BREP Luciferase. To evaluate sDTZ for deep-tissue imaging in the liver, we developed a pAAV-TBG-BREP transfer plasmid featuring a liver-specific promoter. The AAVs were produced and purified following established protocols.26 Subsequently, we administered 100 μL (approximately 1×10¹¹ GC/mL) of AAV-TBG-BREP to mice via tail vein injection. After a 3-week period, the mice were subjected to brightness assessment. Luciferins were dissolved in saline and administered intravenously. All parameters for BLI were consistent with those outlined in the previous section.

BLI of Freely Moving Mice. BALB/cJ mice expressing hippocampal BREP were prepared following the procedure described above. Before imaging, 100 μL of 25 mM sDTZ was intravenously injected into awake mice. The mice were then positioned in the dark box for BLI. The imaging setup was similar to the one described above, with the addition of a TTL device to regulate the camera and an 850 nm LED light source. During each imaging cycle, the LED and camera were activated for 15 ms to capture a brightfield image, immediately followed by a 15 ms frame of bioluminescence with the camera on and LED off. The camera binning and EM gain were adjusted to 8×8 and 1000, respectively. Other parameters remained consistent with the settings detailed above. As large data sets were derived and the system was unable to sustain continuous generation of TTL pulses beyond 8.4 seconds, we recorded separate 8.4-second videos at time intervals of 0, 3, 6, 12, and 18 minutes.

From the data presented herein, Nanoluc and Nanoluc-derived luciferases can tolerate derivatization at the luciferin C2 position. Conjugating sensory chemical functionalities to luciferin through the C2 position enables the generation of sensory luciferins. Although exemplified herein through the sensing of Ca and K⁺ ions additional ions such as Au, Ag, Cu, Zn, and Cl, etc. (e.g., metal ion receptors); and, sulfates, phosphates, additional ions, etc. are expressly contemplated. Additional moieties may take the form of pH sensors, redox-sensitive groups, or enzyme-responsive groups.

As shown by the specific examples above provided, in general, sensory luciferins may be created by the following method. A luciferin may be derivatized at the C2 position of the imidazopyrazinone core with a chemical moiety. The chemical moiety comprises a sensory functionality; wherein the derivatized luciferin is capable of being recognized and utilized by Nanoluc or a Nanoluc-derived luciferase to produce a detectable signal in response to the sensory functionality. The chemical moieties employed include, by way of example, those that can bind Ca2+, Zn2+, Cl−, and other structures that can bind K+ and Na+. The chemical moieties can further include: pH sensors, redox-sensitive groups, or enzyme-responsive groups.

Embodiments contemplated herein may be fabricated through the use of “click” chemistry. For example, the derivatization of luciferin at the C2 position of the imidazopyrazinone core may be performed using click chemistry to conjugate a sensory chemical moiety to the luciferin. In embodiments, the click chemistry may comprise a copper-catalyzed azide-alkyne cycloaddition (CuAAC). In additional embodiments, the click chemistry comprises a strain-promoted azide-alkyne cycloaddition (SPAAC).

In accord with the material herein provided, various aspects of the disclosure may be appreciated by those of ordinary skill in the art. By way of non-limiting examples, various aspects can include at least. A bioluminescent indicator or a metal ion salt thereof having a structure of the formula

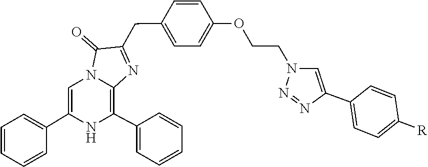

wherein R is

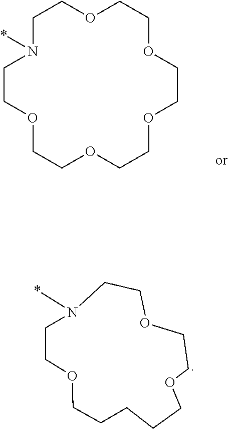 [SMILES: *N1CCOCCOCCOCCOCCOCC1 ; *N1CCOCCCCCOCCOCC1]

In some aspects, the bioluminescent indicator is a monopotassium salt of:

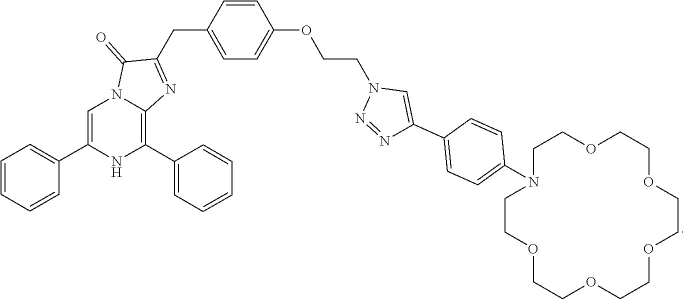 [SMILES: O=c1c(Cc2ccc(OCCn3cc(-c4ccc(N5CCOCCOCCOCCOCCOCC5)cc4)nn3)cc2)nc2c(-c3ccccc3)[nH]c(-c3ccccc3)cn1-2]

In some aspects, the disclosure provides an engineered luciferase protein selected from SEQ ID NO: 46 (BREP), SEQ ID NO: 47 (BRIPO0.5), SEQ ID NO: 48 (BRIPO), or a protein having at least 95% homology to any of these sequences.

Aspects of the disclosure include a method of producing a bioluminescent signal, comprising contacting a monopotassium salt of a bioluminescent indicator of the above formula with an engineered luciferase protein selected from SEQ ID NO: 46 (BREP), SEQ ID NO: 47 (BRIPO0.5), SEQ ID NO: 48 (BRIPO), or a protein having at least 95% homology thereto.

In some aspects, the bioluminescent indicator, upon contact with the engineered luciferase protein BRIPO, exhibits at least a six-fold reduction in bioluminescence intensity when exposed to 150 mM potassium ions compared to the intensity observed in the absence of potassium ions.

In some aspects, the bioluminescent indicator exhibits an apparent dissociation constant (Kd) for potassium ions in a range of about 1 mM to about 100 mM.

In some aspects, the bioluminescent indicator exhibits an apparent dissociation constant (Kd) for sodium ions of at least 50 mM.

Aspects of the disclosure include a bioluminescent calcium indicator composition comprising:
(a) a genetically engineered luciferase fusion protein comprising a NanoLuc-derived luciferase and a red fluorescent protein with at least 80% sequence identity to SEQ ID NO: 49 or SEQ ID NO: 50, wherein the luciferase fusion protein is engineered to include a calcium-binding domain that modulates bioluminescence in response to calcium ion concentration; and (b) a luciferin substrate; wherein, upon binding of calcium ions, the indicator exhibits an increase in red-shifted bioluminescence emission suitable for in vivo imaging of calcium dynamics in mammalian cells or tissues.

In some aspects, the red fluorescent protein is mScarlet-I.

In some aspects, the luciferin substrate is diphenylterazine (DTZ) or a water-soluble derivative thereof.

In some aspects, the indicator exhibits at least a 10-fold increase in bioluminescence intensity at wavelengths greater than 600 nm in response to physiological calcium concentrations.

In some aspects, the indicator is encoded by a nucleic acid and expressed in a mammalian cell or in a transgenic animal.

Aspects of the disclosure include a composition for bioluminescence imaging, comprising a polyethylene glycol (PEG)-conjugated luciferase substrate, wherein the substrate is a coelenterazine (CTZ) or diphenylterazine (DTZ) analog covalently linked to a PEG moiety, and wherein the PEGylated substrate exhibits increased water solubility relative to the non-PEGylated substrate and is suitable for in vivo administration to an animal for bioluminescence imaging with an ATP-independent luciferase.

In some aspects, the PEG moiety has a molecular weight of at least 2,000 Da.

In some aspects, the PEGylated substrate is PEG10 k-DTZ.

In some aspects, the PEGylated substrate is deliverable in normal saline at a concentration of at least 20 mM.

In some aspects, the PEGylated substrate is hydrolyzable in vivo to release the active luciferin.

In some aspects, the composition is used for bioluminescence imaging of brain or liver tissue in a mammal.

In some aspects, the composition enables high-speed video-rate bioluminescence imaging in a freely moving animal.

In some aspects, the luciferin substrate is caged by a caging group at the C3 position of an imidazopyrazinone core.

In some aspects, the PEGylation affects bioluminescence signal kinetics and duration.

In some aspects, the covalent conjugation is via an ester, caronate, or amide linkage.

Aspects of the disclosure include a method of non-invasive bioluminescence imaging in a mammal, comprising administering to the mammal the above composition, expressing an ATP-independent luciferase in a tissue of the mammal, and detecting bioluminescence emission from the tissue.

Aspects of the disclosure include a compound comprising a coelenterazine (CTZ) or diphenylterazine (DTZ) analog, covalently conjugated at the C3 carbonyl group to a cleavable substituent, wherein said substituent is selected from hydrophilic polymers, saccharides, amino acids, peptides, zwitterionic groups, or combinations thereof, wherein the substituent enhances aqueous solubility, stability, pharmacokinetics, or in vivo distribution of the luciferin, and wherein the conjugate is cleavable in vivo to release the luciferin.

Aspects of the disclosure include a compound comprising a coelenterazine (CTZ) or diphenylterazine (DTZ) analog, covalently conjugated at the C3 carbonyl group to a polyethylene glycol (PEG) moiety, wherein said PEG has a molecular weight between 2 kDa and 20 kDa, and wherein the conjugate is cleavable in vivo to release the luciferin.

Aspects of the disclosure include a method for generating a sensory luciferin, comprising derivatizing a luciferin molecule at the C2 position of the imidazopyrazinone core with a chemical moiety, wherein the chemical moiety comprises a sensory functionality, and wherein the derivatized luciferin is capable of being recognized and utilized by NanoLuc or a NanoLuc-derived luciferase to produce a detectable signal in response to the sensory functionality.

In some aspects, the derivatization at the luciferin C2 position of the imidazopyrazinone core is performed using click chemistry to conjugate the sensory chemical moiety to the luciferin.

In some aspects, the click chemistry comprises a copper-catalyzed azide-alkyne cycloaddition (CuAAC).

In some aspects, the click chemistry comprises a strain-promoted azide-alkyne cycloaddition (SPAAC).

In some aspects, the sensory chemical moiety is at least one selected from the group consisting of: metal ion sensors, pH sensors, redox-sensitive groups, and enzyme-responsive groups.

Alternatives. One or more of peptides described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Peptides and/or proteins described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids.

The peptides described herein may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the peptides described herein can include additional residues that may be added at either terminus of a polypeptide for the purpose of providing a “linker” by which the polypeptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present.

In some embodiments, the linker can be a flexible peptide linker that links the peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

The present disclosure provides synthetic nucleic acids, where a subject synthetic nucleic acid comprises a nucleotide sequence encoding a mitochondrial fission inhibitor peptide or construct. A nucleotide sequence encoding a mitochondrial fission inhibitor peptide or construct can be operably linked to one or more regulatory elements, such as a promoter and enhancer, that allow expression of the nucleotide sequence in the intended target cells (e.g., a cell that is genetically modified to synthesize the encoded mitochondrial fission inhibitor construct or peptide). In some embodiments, a subject nucleic acid is a recombinant expression vector.

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, cytomegalovirus immediate early promoter; herpes simplex virus thymidine kinase promoter; early and late SV40 promoters; promoters present in long terminal repeats from a retrovirus; a metallothionein-1 promoter; and the like.

In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, a CYC1 promoter, a HIS3 promoter, an ADHI promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; promoters such as an ssaG promoter or a related promoter (see, e.g., U.S. Patent Publication No. 20040131637), a pagC promoter (Pulkkinen and Miller, J. Bacteriol., 1991: 173 (1): 86-93; Alpuche-Aranda et al., PNAS, 1992; 89 (21): 10079-83), a nirB promoter (Harborne et al. (1992) Mol. Micro. 6:2805-2813), and the like (see, e.g., Dunstan et al. (1999) Infect. Immun. 67:5133-5141; Mckelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and P_{Lambda}. Non-limiting examples of operators for use in bacterial host cells include a lactose promoter operator (LacI repressor protein changes conformation when contacted with lactose, thereby preventing the LacI repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. 80:21-25).

Large numbers of suitable vectors and promoters are known to those of skill in the art; many are commercially available for generating a subject recombinant construct. Large numbers of suitable vectors and promoters are known to those of skill in the art; many are commercially available for generating a subject recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

The present disclosure provides isolated genetically modified host cells (e.g., in vitro cells) that are genetically modified with a nucleic acid comprising a nucleic acid sequence which encodes a mitochondrial fission inhibitor peptide or construct. In some embodiments, a subject isolated genetically modified host cell can produce a mitochondrial fission inhibitor construct or peptide.

Suitable host cells include eukaryotic host cells, such as a mammalian cell, an insect host cell, a yeast cell; and prokaryotic cells, such as a bacterial cell. Introduction of a subject nucleic acid into the host cell can be effected, for example by calcium phosphate precipitation, DEAE dextran mediated transfection, liposome-mediated transfection, electroporation, or other known method.

Suitable yeast cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

Suitable prokaryotic cells include, but are not limited to, any of a variety of laboratory strains of Escherichia coli, Lactobacillus sp., Salmonella sp., Shigella sp., and the like.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all of the subject matter of the above description shown in the accompanying drawings shall be interpreted merely as examples illustrating the inventive concept herein and shall not be construed as limiting the disclosure.

Finally, the written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A bioluminescent indicator or metal ion salt thereof of the formula: 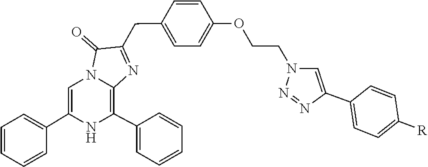
where R is

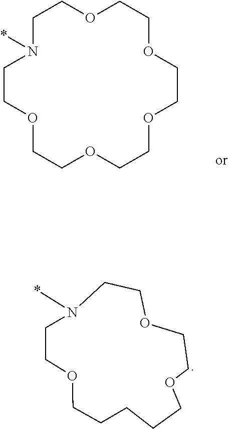 [SMILES: *N1CCOCCOCCOCCOCCOCC1 ; *N1CCOCCCCCOCCOCC1]

2. The bioluminescent indicator of claim 1, wherein the indicator is a monopotassium salt of:

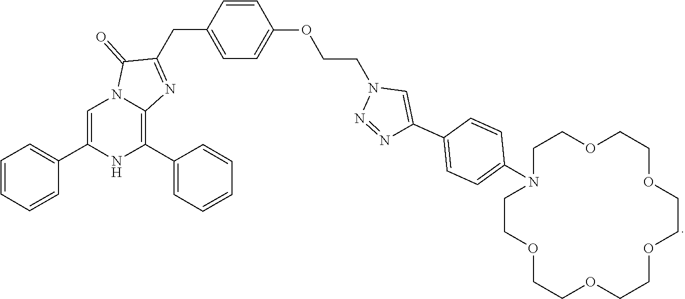 [SMILES: O=c1c(Cc2ccc(OCCn3cc(-c4ccc(N5CCOCCOCCOCCOCCOCC5)cc4)nn3)cc2)nc2c(-c3ccccc3)[nH]c(-c3ccccc3)cn1-2]

3. An engineered luciferase protein of SEQ ID NO: 46 (BREP), SEQ ID NO: 47 (BRIPO0.5), or SEQ ID NO: 48 (BRIPO) or a protein having 95% homology with a protein of SEQ ID NO: 46 (BREP), SEQ ID NO: 47 (BRIPO0.5), or SEQ ID NO: 48 (BRIPO).

4. A method of producing a bioluminescent signal comprising contacting a monopotassium salt of a bioluminescent indicator of the formula 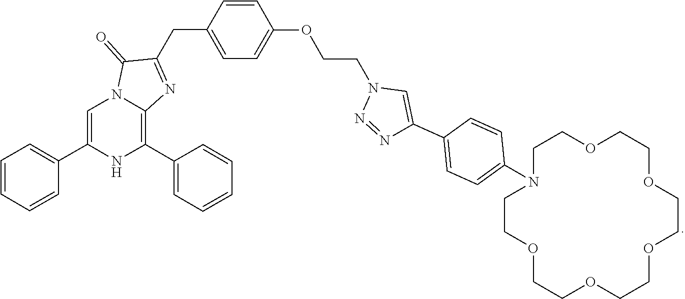 [SMILES: O=c1c(Cc2ccc(OCCn3cc(-c4ccc(N5CCOCCOCCOCCOCCOCC5)cc4)nn3)cc2)nc2c(-c3ccccc3)[nH]c(-c3ccccc3)cn1-2]
with an engineered luciferase protein of SEQ ID NO: 1 (BREP), SEQ ID NO: 2 (BRIPO0.5), or SEQ ID NO: 3 (BRIPO) or a protein having 95% homology with a protein of SEQ ID NO: 46 (BREP), SEQ ID NO: 47 (BRIPO0.5), or SEQ ID NO: 48 (BRIPO).

5. The bioluminescent indicator of claim 1, wherein upon contact with the engineered luciferase protein BRIPO, the indicator exhibits at least a six-fold reduction in bioluminescence intensity when exposed to 150 mM potassium ions compared to the intensity observed in the absence of potassium ions.

6. The bioluminescent indicator of claim 1, wherein the indicator exhibits an apparent dissociation constant (K_{d}) for potassium ions in a range of about 1 mM to about 100 mM.

7. The bioluminescent indicator of claim 1, wherein the indicator exhibits an apparent dissociation constant (K_{d}) for sodium ions of at least 50 mM.

8. A bioluminescent calcium indicator composition comprising:
(a) a genetically engineered luciferase fusion protein comprising a NanoLuc-derived luciferase and a red fluorescent protein with at least 80% or more sequence identity to the proteins with SEQ. ID. NO: 49 or SEQ. ID. NO: 50, wherein the luciferase fusion protein is engineered to include a calcium-binding domain that modulates bioluminescence in response to calcium ion concentration; and,
(b) a luciferin substrate;
wherein, upon binding of calcium ions, the indicator exhibits an increase in red-shifted bioluminescence emission suitable for in vivo imaging of calcium dynamics in mammalian cells or tissues.

9. The composition of claim 8, wherein the red fluorescent protein is mScarlet-I.

10. The composition of claim 8, wherein the luciferin substrate is diphenylterazine (DTZ) or a water-soluble derivative thereof.

11. The composition of claim 8, wherein the indicator exhibits at least a 10-fold increase in bioluminescence intensity at wavelengths greater than 600 nm in response to physiological calcium concentrations.

12. The composition of claim 8, wherein the indicator is encoded by a nucleic acid and expressed in a mammalian cell or in a transgenic animal.

13. A composition for bioluminescence imaging, comprising:
a polyethylene glycol (PEG)-conjugated luciferase substrate, wherein the substrate is a coelenterazine (CTZ) or diphenylterazine (DTZ) analog covalently linked to a PEG moiety, wherein the PEGylated substrate exhibits increased water solubility relative to the non-PEGylated substrate and is suitable for in vivo administration to an animal for bioluminescence imaging with an ATP-independent luciferase.

14. The composition of claim 13, wherein the PEG moiety has a molecular weight of at least 2,000 Da.

15. The composition of claim 13, wherein the PEGylated substrate is PEG10 k-DTZ.

16. The composition of claim 14, wherein the PEGylated substrate is deliverable in normal saline at a concentration of at least 20 mM.

17. The composition of claim 13, wherein the PEGylated substrate is hydrolyzable in vivo to release the active luciferin.

18. The composition of claim 13, wherein the composition is used for bioluminescence imaging of brain or liver tissue in a mammal.

19. The composition of claim 13, wherein the composition enables high-speed video-rate bioluminescence imaging in a freely moving animal.

20. The composition of claim 13, wherein the luciferin substrate is caged by a caging group at the C3 position of an imidazopyrazinone core.

21. The composition of claim 13, wherein the PEGylation affects bioluminescence signal kinetics and duration.

22. The composition of claim 13, wherein the covalent conjugation is via an ester, caronate, or amide linkage.

23. A method of non-invasive bioluminescence imaging in a mammal, comprising:
administering to the mammal the composition of claim 13;
expressing an ATP-independent luciferase in a tissue of the mammal; and,
detecting bioluminescence emission from the tissue.

24. A compound, comprising:
a coelenterazine (CTZ) or diphenylterazine (DTZ) analog, covalently conjugated at the C3 carbonyl group to a cleavable substituent;
wherein said substituent is selected from: hydrophilic polymers, saccharides, amino acids, peptides, zwitterionic groups, or combinations thereof;
wherein the substituent enhances aqueous solubility, stability, pharmacokinetics, or in vivo distribution of the luciferin; and,
wherein the conjugate is cleavable in vivo to release the luciferin.

25. A compound, comprising:
a coelenterazine (CTZ) or diphenylterazine (DTZ) analog, covalently conjugated at the C3 carbonyl group to a polyethylene glycol (PEG) moiety;
wherein, said PEG has a molecular weight between 2 kDa and 20 kDa; and,
wherein the conjugate is cleavable in vivo to release the luciferin.

26. A method for generating a sensory luciferin, comprising:
derivatizing a luciferin molecule at the C2 position of the imidazopyrazinone core with a chemical moiety;
wherein the chemical moiety comprises a sensory functionality;
wherein the derivatized luciferin is capable of being recognized and utilized by Nanoluc or a Nanoluc-derived luciferase to produce a detectable signal in response to the sensory functionality.

27. The method of claim 26, wherein the derivatization at the luciferin C2 position of the imidazopyrazinone core is performed using click chemistry to conjugate the sensory chemical moiety to the luciferin.

28. The method of claim 27, wherein the click chemistry comprises a copper-catalyzed azide-alkyne cycloaddition (CuAAC).

29. The method of claim 27, wherein the click chemistry comprises a strain-promoted azide-alkyne cycloaddition (SPAAC).

30. The method of claim 26 wherein the sensory chemical moiety is at least one selected from the group consisting of: metal ion sensors, pH sensors, redox-sensitive groups, and enzyme-responsive groups.


SEQUENCE LISTING (50 sequences)

SEQ ID NO: 1 - protein, 171 aa, synthetic construct
  REGION 1..171: note = Artificially mutated luciferase polypeptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 2 - protein, 171 aa, synthetic construct
  REGION 1..171: note = Artificially mutated luciferase polypeptide
MVFTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIIDERLINPDGSLLFRVTINGVTGWRLHERILA

SEQ ID NO: 3 - protein, 171 aa, synthetic construct
  REGION 1..171: note = Artificially mutated luciferase polypeptide
KVFTLGDFVGDWRQTAGYNQAQVLEQGGLTSLFQNLGVSVTPIQRIVLSGENGLKIDIHVIIPYEGLSCDQMAQIEKIFKVVYPVDDHHFKAILHYGTLVIDGVTPNMIDYFGQPYEGIAKFDGKKITVTGTLWNGNTIIDERLINPDGSLLFRVTINGVTGWRLHERILA

SEQ ID NO: 4 - DNA, 1185 bp, synthetic construct
  misc_feature 1..1185: note = Artificially mutated luciferase oligonucleotide
  source 1..1185: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggaaagactctcggggattttgttggggactggcgacagacagccggctacaaccaggctcaagtccttgaacagggaggtttgaccagtttgtttcagaacctcggggtgtccgtaactccaatccaaaggattgtcctgagcggtgaaaatgggctgaagatcgatatccatgtcatcatcccgtatgaaggtctgagctgcgaccaaatggcccagatcgaaaaaatttttaaggtggtataccctgtggatgatcatcactttaaggcgatcctgcactatggcacactggtaatcgacggggttacgccgaacatgatcgactatttcggacagccgtatgaaggcatcgccaagttcgacggcaaaaagatcacagtaacagggaccctgtggaacggcaacacaattatcgacgagcgcctgatcaaccccgacggctccctgctgttccgagtaaccattaacggagtgaccggctggcgtctgcatgaacgcattctggcgtaa

SEQ ID NO: 5 - protein, 394 aa, synthetic construct
  REGION 1..394: note = Artificially mutated luciferase polypeptide
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGKTLGDFVGDWRQTAGYNQAQVLEQGGLTSLFQNLGVSVTPIQRIVLSGENGLKIDIHVIIPYEGLSCDQMAQIEKIFKVVYPVDDHHFKAILHYGTLVIDGVTPNMIDYFGQPYEGIAKFDGKKITVTGTLWNGNTIIDERLINPDGSLLFRVTINGVTGWRLHERILA

SEQ ID NO: 6 - protein, 171 aa, synthetic construct
  REGION 1..171: note = Artificially mutated luciferase polypeptide
MVFTLEDFVGDWRQTAGYNQAKALEQGGLTSLFQNLGVSVTPIQRIVLSGENGLKIDIHVIIPYEGLSCDQMAQIEKIFKVVYPVDDHHFKVILHYGTLVIDGVTPNMIDYFGQPYEGIAKFDGKKITVTGTLWNGNKIIDERLINPDGSLLFRVTINGVTGWRLHERILA

SEQ ID NO: 7 - DNA, 1179 bp, synthetic construct
  misc_feature 1..1179: note = Artificially mutated luciferase oligonucleotide
  source 1..1179: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccatcttgatacactcgaagatttcgttggggactggcgacagacagccggctacaacttgagtcaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggataatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcgtctgcatgaacgcattctggcgtaa

SEQ ID NO: 8 - protein, 392 aa, synthetic construct
  REGION 1..392: note = Artificially mutated luciferase polypeptide
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHLDTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIIDERLINPDGSLLFRVTINGVTGWRLHERILA

SEQ ID NO: 9 - DNA, 1962 bp, synthetic construct
  misc_feature 1..1962: note = Artificially mutated luciferase oligonucleotide
  source 1..1962: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaggttggacatcacctcccacaacgaggactacaccgtggtggaacagtacgaacgctccgagggccgcaagcgtcaagatgctagcgaagaagagctatcagagtgctttcgcgtttttgacaaggatggaaatggtttcatcgacagggaggaatttggagacatcatccgccttactggagaacagctcacggatgaggatcccgatgagatatttggagattcagacacagacaaaaatggaaggattgactttgatgagttcctgaagatggtggagaatgtccagtatgttaatattctcaaagtcttcactctcggggattttgttggggactggcgacagacagccggctacaaccaggctcaagtccttgaacagggaggtttgaccagtttgtttcagaacctcggggtgtccgtaactccaatccaaaggattgtcctgagcggtgaaaatgggctgaagatcgatatccatgtcatcatcccgtatgaaggtctgagctgcgaccaaatggcccagatcgaaaaaatttttaaggtggtataccctgtggatgatcatcactttaaggcgatcctgcactatggcacactggtaatcgacggggttacgccgaacatgatcgactatttcggacagccgtatgaaggcatcgccaagttcgacggcaaaaagatcacagtaacagggaccctgataatgcatgaccaactgacagaagagcagattgcagagttcaaagaagccttctcattattcgacaaggatggggacggcaccatcaccacaaaggaacttggcaccgttatgaggtcgcttggacaaaacccaacggaagcagaattgcaggatatgatcaatgaagtcgatgctgatggcaatggaacgatttactttcctgaatttcttactatgatggctagaaaaatgaaggacacagacagcgaagaggaaatccgagaagcattccgtgtttttgacaaggatgggaacggctacatcagcgctgctcagttacgtcacgtcatgacaaacctcggggagaagttaacagatgaagaagttgatgaaatgataagggaagcagatatcgatggtgatggccaagtaaactatgaagagtttgtacaaatgatgacagcaaaggggggcgggtccaagaggcgctggaagaaaaacttcattgccgtcagcgctgccaaccggttcaagaagatctccagctccggggcactggagctttggaacggcaacacaattatcgacgagcgcctgatcaaccccgacggctccctgctgttccgagtaaccattaacggagtgaccggctggcgtctgcatgaacgcattctggcgtaa

SEQ ID NO: 10 - protein, 653 aa, synthetic construct
  REGION 1..653: note = Artificially mutated luciferase polypeptide
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRRLDITSHNEDYTVVEQYERSEGRKRQDASEEELSECFRVFDKDGNGFIDREEFGDIIRLTGEQLTDEDPDEIFGDSDTDKNGRIDFDEFLKMVENVQYVNILKVFTLGDFVGDWRQTAGYNQAQVLEQGGLTSLFQNLGVSVTPIQRIVLSGENGLKIDIHVIIPYEGLSCDQMAQIEKIFKVVYPVDDHHFKAILHYGTLVIDGVTPNMIDYFGQPYEGIAKFDGKKITVTGTLIMHDQLTEEQIAEFKEAFSLFDKDGDGTITTKELGTVMRSLGQNPTEAELQDMINEVDADGNGTIYFPEFLTMMARKMKDTDSEEEIREAFRVFDKDGNGYISAAQLRHVMTNLGEKLTDEEVDEMIREADIDGDGQVNYEEFVQMMTAKGGGSKRRWKKNFIAVSAANRFKKISSSGALELWNGNTIIDERLINPDGSLLFRVTINGVTGWRLHERILA

SEQ ID NO: 11 - DNA, 1962 bp, synthetic construct
  misc_feature 1..1962: note = Artificially mutated luciferase oligonucleotide
  source 1..1962: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaggttggacatcacctcccacaacgaggactacaccgtggtggaacagtacgaacgctccgagggccgcaagcgtcaagatgctagcgaagaagagctatcagagtgctttcgcgtttttgacaaggatggaaatggtttcatcgacagggaggaatttggagacatcatccgccttactggagaacagctcacggatgaggatcccgatgagatatttggagattcagacacagacaaaaatggaaggattgactttgatgagttcctgaagatggtggagaatgtccagtatgttaatattctcatggtcttcacactcgaagatttcgttggggactggcgacagacagccggctacaacttgagtcaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggataatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgataatgcatgaccaactgacagaagagcagattgcagagttcaaagaagccttctcattattcgacaaggatggggacggcaccatcaccacaaaggaacttggcaccgttatgaggtcgcttggacaaaacccaacggaagcagaattgcaggatatgatcaatgaagtcgatgctgatggcaatggaacgatttactttcctgaatttcttactatgatggctagaaaaatgaaggacacagacagcgaagaggaaatccgagaagcattccgtgtttttgacaaggatgggaacggctacatcagcgctgctcagttacgtcacgtcatgacaaacctcggggagaagttaacagatgaagaagttgatgaaatgataagggaagcagatatcgatggtgatggccaagtaaactatgaagagtttgtacaaatgatgacagcaaaggggggcgggtccaagaggcgctggaagaaaaacttcattgccgtcagcgctgccaaccggttcaagaagatctccagctccggggcactggagctttggaacggcaacaaaattatcgacgagcgcctgatcaaccccgacggctccctgctgttccgagtaaccatcaacggagtgaccggctggcgtctgcatgaacgcattctggcgtaa

SEQ ID NO: 12 - protein, 653 aa, synthetic construct
  REGION 1..653: note = Artificially mutated luciferase polypeptide
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRRLDITSHNEDYTVVEQYERSEGRKRQDASEEELSECFRVFDKDGNGFIDREEFGDIIRLTGEQLTDEDPDEIFGDSDTDKNGRIDFDEFLKMVENVQYVNILMVFTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLIMHDQLTEEQIAEFKEAFSLFDKDGDGTITTKELGTVMRSLGQNPTEAELQDMINEVDADGNGTIYFPEFLTMMARKMKDTDSEEEIREAFRVFDKDGNGYISAAQLRHVMTNLGEKLTDEEVDEMIREADIDGDGQVNYEEFVQMMTAKGGGSKRRWKKNFIAVSAANRFKKISSSGALELWNGNKIIDERLINPDGSLLFRVTINGVTGWRLHERILA

SEQ ID NO: 13 - DNA, 936 bp, synthetic construct
  misc_feature 1..936: note = Artificially mutated luciferase oligonucleotide
  source 1..936: mol_type = other DNA
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 14 - protein, 311 aa, synthetic construct
  REGION 1..311: note = Artificially mutated luciferase polypeptide
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 15 - DNA, 1653 bp, synthetic construct
  misc_feature 1..1653: note = Artificially mutated luciferase oligonucleotide
  source 1..1653: mol_type = other DNA
atggaagatgccaaaaacattaagaagggcccagcgccgttctacccactcgaagacgggaccgccggcgagcagctgcacaaagccatgaagcgctacgccctggtgcccggcgccatcgcctttaccgacgcacatattcaggtggacgttacctacgccgagtacttcgagatgagcgttcggctggcagaagctatgaggcgctatgggctgaatacaaaccatcggatcgtggtgtgcagcgagaatagctcgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcgaaaggtcctcaacgtgcaaaagaagctaccgatcatacgaaagatcatcatcatggatagcaagaccgactaccagggcttccaaagcatgtacaccttcgtgacttcccatttgccacccagcttcaacgagtacgacttcgtgcccgagagcttcgaccgggacaaaaccatcgccctgatcatgaacagtagtggtagtacaggattacccaagggcgtagccctaccgcaccgcaccgcttgtgtccgattcagtcatgcccgcgaccccatcttcggctaccagaacatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagctgcctcgctaagagcactctcatcgacaagtacgacctaagcagcttgcgcgagatcgccagcggcggggcgccgctcagcaaggaggtaggtgaggccgtggccaaacgcttccgcctaccaggcatccgccagggctatggcctgacagaaacaaccaacgccgtcatgatcacccccgagggggaccgtaagcctggctcagtaggcaaggtggtgcccttcttcgaggctaaggtggtagacttggtcaccggtaagacactgggtgtgaaccagcgcggtgagctgtgcgtccgtggccccatgatcatgagcggctacgttaacaaccccgaggctacgaacgctctcatcgacaaggacggctggctgcacagcggcgacatcgcctactgggacgaggacgagcacttcttcatcgtggaccggctgaagagcctgatcaaatacaagggctaccaggtagccccagccgaactggagggcatcctgctgcaacacccctacatcttcgacgccggagtcgccggcctgcccgacgacgatgccggcgagctgcccgccgcagtcgtcgtgttggaacacggtaaaaccatgaccgagaaagagatcgtggactatgtggccagccaggttacaaccgccaagaagctgcgcggtggtgttgtgtttgtggatgaagtccctagaggatcgaccggcaagttagacgcccgcaagatccgcgagattctcactaaggccaagaaggacggcaagatcgccgtgtaa

SEQ ID NO: 16 - protein, 550 aa, synthetic construct
  REGION 1..550: note = Artificially mutated luciferase polypeptide
MEDAKNIKKGPAPFYPLEDGTAGEQLHKAMKRYALVPGAIAFTDAHIQVDVTYAEYFEMSVRLAEAMRRYGLNTNHRIVVCSENSSQFFMPVLGALFIGVAVAPANDIYNERELLNSMGISQPTVVFVSKKGLRKVLNVQKKLPIIRKIIIMDSKTDYQGFQSMYTFVTSHLPPSFNEYDFVPESFDRDKTIALIMNSSGSTGLPKGVALPHRTACVRFSHARDPIFGYQNIPDTAILSVVPFHHGFGMFTTLGYLICGFRVVLMYRFEEELFLRSLQDYKIQSALLVPTLFSCLAKSTLIDKYDLSSLREIASGGAPLSKEVGEAVAKRFRLPGIRQGYGLTETTNAVMITPEGDRKPGSVGKVVPFFEAKVVDLVTGKTLGVNQRGELCVRGPMIMSGYVNNPEATNALIDKDGWLHSGDIAYWDEDEHFFIVDRLKSLIKYKGYQVAPAELEGILLQHPYIFDAGVAGLPDDDAGELPAAVVVLEHGKTMTEKEIVDYVASQVTTAKKLRGGVVFVDEVPRGSTGKLDARKIREILTKAKKDGKIAV

SEQ ID NO: 17 - protein, 171 aa, synthetic construct
  REGION 1..171: note = Artificially mutated luciferase polypeptide
EFFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 18 - protein, 171 aa, synthetic construct
  REGION 1..171: note = Artificially mutated luciferase polypeptide
MVLTLEDFVGDWRQTDGYNQAQVLEQGGLTSLFQNLGVSVTPIQRIVLSGENGLKIDIHVIIPYEGLSGDRMGQIEKIFKVVYPVDDHHFKVILHYGTLVIDGVTPNMIDYFGQPYEGIAVFDGKKITVTGTLWNGNKIIDERRINPDGSLLFRVTINGVTGWRLSERILA

SEQ ID NO: 19 - DNA, 20 bp, synthetic construct
  misc_feature 1..20: note = Artificial oligonucleotide promoter
  source 1..20: mol_type = other DNA
atgccatagcatttttatcc

SEQ ID NO: 20 - DNA, 18 bp, synthetic construct
  misc_feature 1..18: note = Artificial oligonucleotide promoter
  source 1..18: mol_type = other DNA
gatttaatctgtatcagg

SEQ ID NO: 21 - DNA, 29 bp, synthetic construct
  misc_feature 1..29: note = Artificial oligonucleotide promoter
  source 1..29: mol_type = other DNA
taccgagctcatccagtttggactagtgg

SEQ ID NO: 22 - DNA, 31 bp, synthetic construct
  misc_feature 1..31: note = Artificial oligonucleotide promoter
  source 1..31: mol_type = other DNA
agcccagatctcctctagagtctagatctgg

SEQ ID NO: 23 - DNA, 52 bp, synthetic construct
  misc_feature 1..52: note = Artificial oligonucleotide promoter
  source 1..52: mol_type = other DNA
aaagccaccggcgcgccgccgccaccatggtcttcactctcgaagattttgt

SEQ ID NO: 24 - DNA, 38 bp, synthetic construct
  misc_feature 1..38: note = Artificial oligonucleotide promoter
  source 1..38: mol_type = other DNA
tcgaagcggccggccttacgccagaatgcgttcatgca

SEQ ID NO: 25 - DNA, 51 bp, synthetic construct
  misc_feature 1..51: note = Artificial oligonucleotide promoter
  source 1..51: mol_type = other DNA
aaagccaccggcgcgccgccgccaccatggaagatgccaaaaacattaaga

SEQ ID NO: 26 - DNA, 42 bp, synthetic construct
  misc_feature 1..42: note = Artificial oligonucleotide promoter
  source 1..42: mol_type = other DNA
tcgaagcggccggccttacacggcgatcttgccgtccttctt

SEQ ID NO: 27 - DNA, 48 bp, synthetic construct
  misc_feature 1..48: note = Artificial oligonucleotide promoter
  source 1..48: mol_type = other DNA
aaagccaccggcgcgccgccgccaccatggcttccaaggtgtacgacc

SEQ ID NO: 28 - DNA, 41 bp, synthetic construct
  misc_feature 1..41: note = Artificial oligonucleotide promoter
  source 1..41: mol_type = other DNA
tcgaagcggccggccttactgctcgttcttcagcacgcgct

SEQ ID NO: 29 - DNA, 48 bp, synthetic construct
  misc_feature 1..48: note = Artificial oligonucleotide promoter
  source 1..48: mol_type = other DNA
  variation 19..20: note = n is a, c, g, t or u
  variation 22..23: note = n is a, c, g, t or u
cagacagccggctacaacnnknnkcaagtccttgaacagggaggtgtg

SEQ ID NO: 30 - DNA, 48 bp, synthetic construct
  misc_feature 1..48: note = Artificial oligonucleotide promoter
  source 1..48: mol_type = other DNA
  variation 26..27: note = n is a, c, g, t or u
  variation 29..30: note = n is a, c, g, t or u
cacacctccctgttcaaggacttgmnnmnngttgtagccggctgtctg

SEQ ID NO: 31 - DNA, 51 bp, synthetic construct
  misc_feature 1..51: note = Artificial oligonucleotide promoter
  source 1..51: mol_type = other DNA
  variation 22..23: note = n is a, c, g, t or u
  variation 25..26: note = n is a, c, g, t or u
  variation 28..29: note = n is a, c, g, t or u
caagtccttgaacagggaggtnnknnknnkttgtttcagaatctcggggtg

SEQ ID NO: 32 - DNA, 51 bp, synthetic construct
  misc_feature 1..51: note = Artificial oligonucleotide promoter
  source 1..51: mol_type = other DNA
  variation 23..24: note = n is a, c, g, t or u
  variation 26..27: note = n is a, c, g, t or u
  variation 29..30: note = n is a, c, g, t or u
caccccgagattctgaaacaamnnmnnmnnacctccctgttcaaggacttg

SEQ ID NO: 33 - DNA, 20 bp, synthetic construct
  misc_feature 1..20: note = Artificial oligonucleotide promoter
  source 1..20: mol_type = other DNA
atgccatagcatttttatcc

SEQ ID NO: 34 - DNA, 18 bp, synthetic construct
  misc_feature 1..18: note = Artificial oligonucleotide promoter
  source 1..18: mol_type = other DNA
gatttaatctgtatcagg

SEQ ID NO: 35 - DNA, 43 bp, synthetic construct
  misc_feature 1..43: note = Artificial oligonucleotide promoter
  source 1..43: mol_type = other DNA
aataaagcttgccgccaccatggtcttcactctcggggatttt

SEQ ID NO: 36 - DNA, 35 bp, synthetic construct
  misc_feature 1..35: note = Artificial oligonucleotide promoter
  source 1..35: mol_type = other DNA
taattctcgagttacgccagaatgcgttcatgcag

SEQ ID NO: 37 - DNA, 46 bp, synthetic construct
  misc_feature 1..46: note = Artificial oligonucleotide promoter
  source 1..46: mol_type = other DNA
attataaagcttgccgccaccatggaagatgccaaaaacattaaga

SEQ ID NO: 38 - DNA, 36 bp, synthetic construct
  misc_feature 1..36: note = Artificial oligonucleotide promoter
  source 1..36: mol_type = other DNA
ataactcgagcatggaagatgccaaaaacattaaga

SEQ ID NO: 39 - DNA, 36 bp, synthetic construct
  misc_feature 1..36: note = Artificial oligonucleotide promoter
  source 1..36: mol_type = other DNA
ttgccaagcttacacggcgatcttgccgtccttctt

SEQ ID NO: 40 - DNA, 44 bp, synthetic construct
  misc_feature 1..44: note = Artificial oligonucleotide promoter
  source 1..44: mol_type = other DNA
attataaagcttgccgccaccatggtgagcaagggcgaggcagt

SEQ ID NO: 41 - DNA, 35 bp, synthetic construct
  misc_feature 1..35: note = Artificial oligonucleotide promoter
  source 1..35: mol_type = other DNA
ataactcgagcatggtgagcaagggcgaggcagtg

SEQ ID NO: 42 - DNA, 32 bp, synthetic construct
  misc_feature 1..32: note = Artificial oligonucleotide promoter
  source 1..32: mol_type = other DNA
ttgccaagcttacgccagaatgcgttcatgca

SEQ ID NO: 43 - DNA, 45 bp, synthetic construct
  misc_feature 1..45: note = Artificial oligonucleotide promoter
  source 1..45: mol_type = other DNA
  variation 22..23: note = n is a, c, g, t or u
gagggccgccactccaccggannkactctcggggattttgttggg

SEQ ID NO: 44 - DNA, 45 bp, synthetic construct
  misc_feature 1..45: note = Artificial oligonucleotide promoter
  source 1..45: mol_type = other DNA
  variation 23..24: note = n is a, c, g, t or u
cccaacaaaatccccgagagtmnntccggtggagtggcggccctc

SEQ ID NO: 45 - DNA, 38 bp, synthetic construct
  misc_feature 1..38: note = Artificial oligonucleotide promoter
  source 1..38: mol_type = other DNA
ttattctcgagttacacggcgatcttgccgtccttctt

SEQ ID NO: 46 - protein, 392 aa, synthetic construct
MVSKGEAVIKEFMRFKVHMEGSMNGHEFEIGEGEGGRPYEEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTIYKAKKPVQMPGAYNVDRKLDITSHNEDYTVVEQYERSEGRHLDTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIDERLINPDGSLLFRVTINGVTGWRLHERILA

SEQ ID NO: 47 - protein, 392 aa, synthetic construct
MVSKGEAVIKEFMRFKVHMEGSMNGHGFEIGEGEGGRPYEEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTIYKAKKPVQMPGAYNVDRKLDITSHNEDYTVVEQYERSEGRHLVTLEDFVGDWRQTAGYNLSQVLEQGGVSSLFQNLGVSVTPIQRIVLSGENGLKIGIHVIIPYEGLSGDQMGQIEKIFKVVYPVDNHHFKVILHYGTLVIDGVTPNMIDYFGRPYEGIAVFDGKKITVTGTLWNGNKIDERLINPDGSLLFRVTINGVTGWRLHERILA

SEQ ID NO: 48 - protein, 392 aa, synthetic construct
MVSKGEAVIKEFMRFKVHMEGSMNGHGFEIGEGEGGRPYEEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTIYKAKKPVQMPGAYNVDRKLDITSHNEDYTVVEQYERSEGRHLVTLEDFVGDWRQTAGYNLSQVLEQGGVSSLFQNLGVRWTPIQRIVLSGENGLKIGIHVIIPYEGLSGDQMGQIEKIFKVVYPVDNHHFKVILHYGTLVIDGVTPNMIDYFGRPYEGIAVFDGKKITVTGTLWNGNKIDERLINPDGSLLFRVTINGVTGWRLHERILA

SEQ ID NO: 49 - protein, 574 aa, synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTIYKAKKPVQMPGAYNVDRKLDITSHNEDYTVVEQYERSEGRHLDTLEDFVGDWRQTAGYNLSQILEQGGVSSLFQNLGVSVTPIQRIVLSGENGLKIDIHVIIPYEGLSGDQMGQIEKIFKVVYPVDNHHFKVILHYGTLVIDGVTPNLIDYFGRPYEGIAVFDGKKITVTGTLIMHDQLTEEQIAEFKEAFSLFDKDGDGTITTKELGTVMRSLGQNPTEAELQDMINEVDADGNGTIYFPEFLTMMARKMKDTDSEEEIREAFRVFDKDGNGYISAAQLRHVMTNLGEKLTDEEVDEMIREADIDGDGQVNYEEFVQMMTAKGGESKRRWKKNFIAVSAANRFKKISSSGALELWKGNKIIDERLINPDGSLLFRVTINGVTGWRLHERILA

SEQ ID NO: 50 - protein, 574 aa, synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYEVKLRGTNFPPDGPVMQKKTMGWEASTERLYPEDGVLKGDIKMALRLKDGGRYLADFKTIYKAKKPVQMPGAYNVDRKLDITSHNEDYTVVEQYERSEGRHLDTLEVFVGDWRQTAGYNLSQILEQGGVSSLFQNLGVSVTPIQRIVRSGENGLKIDIHVIIPYEGLSGDQMGQIEKIFKVVYPVDNHHFKVILHYGTLVIDGVTPNLIDYFGRPYEGIAVFDGKKITVTGTLIMHDQLTEEQIAEFKEAFSLFDKDGDGTITTKELGTVMRSLGQNPTEAELQDMINEVDADGNGTIYFPEFLTMMARKMKDTDSEEEIREAFRVFDKDGNGYISAAQLRHVMTNLGEKLTEEEVDEMIREADIDGDGQVNYEEFVQMMTAKGGESKRRWKKDFIAVSAANRFKKISSSGALELWKGNKIIDERLINPDGSLLFRVTINGVTGWRLHERILA